(19) United States
(12) Patent Application Publication
(10) Pub. No.: US 2006/0182714 A1
(43) Pub. Date: Aug. 17, 2006

(54) Synthesis and application of new structural well defined branched polymers as conjugating agents for peptides

(75) Inventors: Carsten Behrens, Kobenhavn N (DK); Florencio Zaragoza Dorwald, Smorum (DK); Mikael Kofod-Hansen, Kobenhavn N (DK); Jesper Lau, Farum (DK); Janos Tibor Kodra, Kobenhavn O (DK); Thomas Kruse Hansen, Herlev (DK); Paw Bloch, Taastrup (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/344,767

(22) Filed: Feb. 1, 2006

Related U.S. Application Data
(63) Continuation of application No. PCT/DK04/00531, filed on Aug. 9, 2004.

(30) Foreign Application Priority Data
Aug. 8, 2003 (DK) PA 2003 01145
Nov. 5, 2003 (DK) PA 2003 01646

(51) Int. Cl. A61K 39/395; A61K 38/28; A61K 38/22; A61K 38/20; C07K 14/62; C07K 14/54; C07K 16/46

(52) U.S. Cl. 424/85.2; 424/178.1; 514/12; 530/351; 530/397; 530/391.1; 530/303; 514/3; 530/383

(57) ABSTRACT

The invention provides synthesis and application of new structural well defined branched polymers as protraction agents for peptide and protein.

### CROSS-REFERENCE TO RELATED APPLICATIONS

This application is a continuation of International Application No. PCT/DK2004/000531, filed Aug. 9, 2004, which claims priority from Danish Patent Application Nos. PA 2003 01145 filed Aug. 8, 2003; PA 2003 01646 filed Nov. 5, 2003 and to U.S. Patent Application Nos. 60/494,447 filed Aug. 12, 2003 and 60/519,212 filed Nov. 12, 2003.

### FIELD OF INVENTION

This invention relates to the synthesis of new structural well defined branched polymers prepared using a precise number of monomer units, and the application of such branched polymers as protracting agents for pharmaceutical peptides. More particular, the present invention relates to methods for chemically modifying target molecules e.g. macromolecules, in particularly biological important peptides, by covalent attachment of structural well defined branched polymers made from a precise number of monomer units, aiming for improving their pharmacokineticor pharmacodynamical properties.

### BACKGROUND OF THE INVENTION

Peptides of therapeutic interest such as hormones, soluble receptors, cytokines, enzymes etc. often have short circulation half-life in the body as a result of proteolytical degradation, clearance by the kidney or liver, or in some cases the appearance of neutralizing antibodies. This generally reduces the therapeutic utility of peptides.

It is however well recognised that the properties of peptides can be enhanced by grafting organic chain-like molecules onto them. Such grafting can improve pharmaceutical properties such as half life in serum, stability against proteolytical degradation, and reduced immunogenicity.

The organic chain-like molecules often used to enhance properties are polyethylene glycol-based or “PEG-based” chains, i.e., chains that are based on the repeating unit —CH₂CH₂O—. However, the techniques used to prepare PEG or PEG-based chains, even those of fairly low molecular weight, involve a poorly-controlled polymerisation step which leads to preparations having a wide spread of chain lengths about a mean value. Consequently, peptide conjugates based on PEG grafting are generally characterised by broad range molecular weight distributions.

Kochendoefer et al. recently described (Science 2003, 299, 884-887) the design and synthesis of a homogeneous polymer modified erythropoiesis protein, and in WO02/20033 devised a general method for the synthesis of well defined polymer modified peptides. The building blocks used in this work were based on alternating water soluble linear long chain hydrophilic diamines and succinate, which were extended by sequential addition using standard peptide chemistry in solution or on solid support.

An alternative and more attractive strategy for preparing large well defined polymers in a minimum of synthesis steps, relies on the use of bi-, tri or multi-furcated monomers in a limit number of sequential oligomerisation steps. The mass growth of the polymer will in this case follow an exponential curve, with an exponent determined by the furcation number, e.g. bifurcated monomers provides 2th power growth, trifurcated monomers 3th power growth etc. The type of polymers obtained by this procedure has been well described in the literature (S. M. Grayson and J. M. J. Frechet, Chem Rev. 2001, 101, 3819) and are commonly known as dendrimers.

Biodegradable 4th generation polyester dendrimers based on 2,2-bis(hydroxymethyl)-propionic acid and capped with polyethyleneoxide via a carbamate linkage has recently been reported (E. R. Gillies and J. M. J. Frechet, J. Amer. Chem. Soc, 2002, 124, 14137-14146). The architecture of this system bears a close resemblance to the system described by Kochendoefer et al. as described above, as the dendritic part of the structure is used to generate a polyhydroxy scaffold that function as attachment points for the capped polyethyleneoxide tails. Although impressive 12 KD structures can be made, no further extension of the ethylene oxide part of the structure is possible.

In light of the many potential applications for well defined polymer conjugated to biopharmaceuticals (e.g. modifying pharmacokinetics and pharmacodynamics), there is a continuous need in the art for improving the technology for preparing well defined polymers and copolymers in a precise well defined manner, from a precise number of monomer units.

### SUMMARY OF THE INVENTION

The present invention provides a a new class of branched polymers, and the conjugation of such branched polymers to polypeptides and a method of producing the branched polymers and the conjugates. It also provides a method for direct modification of solid phase bounded polypeptides, by combining standard solid phase peptide synthesis, with on resin oligomerisation of monomers described according to the invention into branched polymers. The invention provides a method of constructing a polypeptide on solid support, and furnish it with a branched polymer of precise size with respect to number of monomer building blocks, and types of these, whether it be linear or branched monomers.

Thus, the invention provides a conjugate comprising a mono disperse branched polymer covalently attached to a peptide.

The invention also provides a pharmaceutical composition comprising at least one conjugate as described above together with pharmaceutical acceptable carriers and diluents.

The invention also provides a method for producing a conjugate as above by attachment of one or more reactive derivative of the branched polymer to attachment groups on the peptide.

The invention also provides the use of a conjugate as above as a medicament. The invention provides the branched polymers comprised in the conjugates above. The invention provides a method for producing such branched polymers by two different approaches.

Definitions

The term “covalent attachment” means that the polymeric molecule and the peptide is either directly covalently joined to one another, or else is indirectly covalently joined to one another through an intervening moiety or moieties, such as bridge, spacer, or linkage moiety or moieties.

The term “conjugate”, or “conjugate peptide”, is intended to indicate a heterogeneous (in the sense of composite or chimeric) molecule formed by covalent attachment of one or more peptides to one or more polymer molecules.

The term “peptide” or “protein” encompasses any peptide of either natural or synthetic origin, that consist of any number of amino acids having at least 2 residues. Also the product from ligation of two or more peptide fragments are considered in this context, the ligation process resulting in either native peptide bonds, or synthetic chemical bonds such as oximes or peptidomimics. Also the use of peptide fragments containing unnatural amino acid residues are considered in this context.

“Immunogenicity” of a polymer modified peptide refers to the ability of the polymer modified peptide, when administrated to a human, to elicit an immune response, whether humoral, cellular, or both.

The term “attachment group” is intended to indicate a functional group on the peptide or a linker modified peptide capable of attaching a polymer molecule either directly or indirectly through a linker. Useful attachment groups are, for example, amine, hydroxyl, carboxyl, aldehyde, ketone, sulfhydryl, succinimidyl, maleimide, vinylsulfone or haloacetate.

The term “branched polymer”, or “dendritic polymer” or “dendritic structure” means an organic polymer assembled from a selection of monomer building blocks of which, some contains branches.

The term “reactive functional group” means by way of illustration and not limitation, any free amino, carboxyl, thiol, alkyl halide, acyl halide, chloroformiate, aryloxycarbonate, hydroxy or aldehyde group, carbonates such as the p-nitrophenyl, or succinimidyl; carbonyl imidazoles, carbonyl chlorides; carboxylic acids that are activated in situ; carbonyl halides, activated esters such as N-hydroxysuccinimide esters, N-hydroxybenzotriazole esters, esters of such as those comprising 1,2,3-benzotriazin-4(3H)-one, phosphoramidites and H-phosphonates, phosphortriesters or phosphordiesters activates in situ, isocyanates or isothiocyanates, in addition to groups such as NH₂, OH, N₃, NHR′, OR′, O—NH₂, alkynes, or any of the following

hydrazine derivatives —NH—NH₂,

hydrazine carboxylate derivatives —O—C(O)—NH—NH₂,

semicarbazide derivatives —NH—C(O)—NH—NH₂,

thiosemicarbazide derivatives —NH—C(S)—NH—NH₂,

carbonic acid dihydrazide derivatives —NHC(O)—NH—NH—C(O)—NH—NH₂,

carbazide derivatives —NH—NH—C(O)—NH—NH₂,

thiocarbazide derivatives —NH—NH—C(S)—NH—NH₂,

aryl hydrazine derivatives —NH—C(O)—C₆H₄—NH—NH₂,

hydrazide derivatives —C(O)—NH—NH₂; and

oxylamine derivatives, such as —C(O)—O—NH₂, —NH—C(O)—O—NH₂ and —NH—C(S)—O—NH₂

The term “protected functional group” means a functional group which has been protected in a way rendering it essential non-reactive. Examples for protection groups used for amines includes but is not limited to tert-butoxycarbonyl, 9-fluorenylmethyloxycarbonyl, azides etc. For a carboxyl group other groups becomes relevant such as tert-butyl, or more generally alkyl groups. Appropriate protection groups are known to the skilled person, and examples can be found in Green & Wuts “Protection groups in organic synthesis”, 3.ed. Wiley-interscience.

The term “cleavable moiety” is intended to mean a moiety that is capable of being selectively cleaved to release the branched polymer based linker or branched polymer linker based peptide from the solid support.

The term “generation” means a single uniformly layer, created by reacting one or more identical functional groups on a organic molecule with a particular monomer building block. With a branched polymer made from exclusively bifurcated monomers, the number of reactive groups in a generation is given by the formula (2*(m−1))², where m is an integer of 1, 2, 3 . . . 8 representing the particular generation. For a branched polymer made from exclusively trifurcated monomers, the number of reactive groups is given by the formula (3*(m−1))³, and for a branched polymer made exclusively from a multifurcated monomer with n-branches, the number of reactive groups is given by (n*(m−1))ⁿ. For branched polymers in which different monomers are used in each individual generation, the number of reactive groups in a particular layer or generation can be calculated recursively knowing the layer position and the number of branches of the individual monomers.

The term “functional in vivo half-life” is used in its normal meaning, i.e., the time at which 50% of the biological activity of the peptide or conjugate is still present in the body/target organ, or the time at which the activity of the peptide or conjugate is 50% of its initial value. As an alternative to determining functional in vivo half-life, “serum half-life” may be determined, i.e., the time at which 50% of the peptide or conjugate molecules circulate in the plasma or bloodstream prior to being cleared. Determination of serum-half-life is often more simple than determining functional half-life and the magnitude of serum-half-life is usually a good indication of the magnitude of functional in vivo half-life. Alternative terms to serum half-life include plasma half-life, circulating half-life, circulatory half-life, serum clearance, plasma clearance, and clearance half-life. The peptide or conjugate is cleared by the action of one or more of the reticulo-endothelial system (RES), kidney, spleen, or liver, by tissue factor, SEC receptor, or other receptor-mediated elimination, or by specific or unspecific proteolysis. Normally, clearance depends on size (relative to the cut-off for glomerular filtration), charge, attached carbohydrate chains, and the presence of cellular receptors for the peptide. The functionality to be retained is normally selected from procoagulant, proteolytic, co-factor binding or receptor binding activity. The functional in vivo half-life and the serum half-life may be determined by any suitable method known in the art.

The term “increased” as used about the functional in vivo half-life or plasma half-life is used to indicate that the relevant half-life of the peptide or conjugate is statistically significantly increased relative to that of a reference molecule, for example such as non-conjugated Factor Vila (e.g., wild-type FVIIa) as determined under comparable conditions. For instance the relevant half-life may be increased by at least about 10% or at least 25%, such as by at least about 50%, e.g., by at least about 100%, 150%, 200%, 250%, or 500%.

The term “halogen” means F, Cl, Br or I.

The terms “alkyl” or “alkylene” refer to a C₁₋₆-alkyl or -alkylene, representing a saturated, branched or straight hydrocarbon group having from 1 to 6 carbon atoms. Typical C₁₋₆-alkyl groups include, but are not limited to, methyl, ethyl, n-propyl, isopropyl, butyl, isobutyl, sec-butyl, tert-butyl, pentyl, hexyl and the corresponding divalent radicals.

The terms “alkenyl” or “alkenylene” refer to a C₂₋₆-alkenyl or -alkenylene, representing a branched or straight hydrocarbon group having from 2 to 6 carbon atoms and at least one double bond. Typical C₂₋₆-alkenyl groups include, but are not limited to, ethenyl, 1-propenyl, 2-propenyl, isopropenyl, 1,3-butadienyl, 1-butenyl, 2-butenyl, 1-pentenyl, 2-pentenyl, 1-hexenyl, 2-hexenyl, 1-ethylprop-2-enyl, 1,1-(dimethyl)prop-2-enyl, 1-ethylbut-3-enyl, 1,1-(dimethyl)but-2-enyl, and the corresponding divalent radicals.

The terms “alkynyl” or “alkynylene” refer to a C₂₋₆-alkynyl or -alkynylene, representing a branched or straight hydrocarbon group having from 2 to 6 carbon atoms and at least one triple bond. Typical C₂₋₆-alkynyl groups include, but are not limited to, vinyl, 1-propynyl, 2-propynyl, isopropynyl, 1,3-butadynyl, 1-butynyl, 2-butynyl, 1-pentynyl, 2-pentynyl, 1-hexynyl, 2-hexynyl, 1-ethylprop-2-ynyl, 1,1-(dimethyl)prop-2-ynyl, 1-ethylbut-3-ynyl, 1,1-(dimethyl)but-2-ynyl, and the corresponding divalent radicals.

The terms “alkyleneoxy” or “alkoxy” refer to “C₁₋₆-alkoxy” or -alkyleneoxy representing the radical —O—C₁₋₆-alkyl or —O—C₁₋₆-alkylene, wherein C₁-alkyl(ene) is as defined above. Representative examples are methoxy, ethoxy, n-propoxy, isopropoxy, butoxy, sec-butoxy, tert-butoxy, pentoxy, isopentoxy, hexoxy, isohexoxy and the like.

The terms “alkylenethio”, “alkenylenethio” or “alkynylenethio”; refer to the corresponding thio analogues of the oxy-radicals as defined above. Representative examples are methylthio, ethylthio, propylthio, butylthio, pentylthio, hexylthio, and the corresponding divalent radicals and the corresponding alkenyl and alkynyl derivatives also defined above.

In the context of this invention the term “-triyl” is used and refers to different alkyl, alkenyl, alkynyl, cycloalkyl or aromatic radicals with three attachment points.

The term “cycloalkyl” refers to C₃₋₈-cycloalkyl representing a monocyclic, carbocyclic group having from from 3 to 8 carbon atoms. Representative examples are cyclopropyl, cyclobutyl, cyclopentyl, cyclohexyl, cycloheptyl, cyclooctyl, and the like.

The term “cycloalkenyl” refers to C₃₋₈-cycloalkenyl representing a monocyclic, carbocyclic, non-aromatic group having from 3 to 8 carbon atoms and at least one double bond. Representative examples are cyclopropenyl, cyclobutenyl, cyclopentenyl, cyclohexenyl, cycloheptenyl, cyclooctenyl and the like.

The term “aryl” as used herein is intended to include carbocyclic aromatic ring systems such as phenyl, biphenylyl, naphthyl, anthracenyl, phenanthrenyl, fluorenyl, indenyl, pentalenyl, azulenyl and the like. Aryl is also intended to include the partially hydrogenated derivatives of the carbocyclic systems enumerated above. Non-limiting examples of such partially hydrogenated derivatives are 1,2,3,4-tetrahydronaphthyl, 1,4-dihydronaphthyl and the like.

The term “heteroaryl” as used herein is intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term heteroaryl-C₁₋₆-alkyl as used herein denotes heteroaryl as defined above and C₁₋₆-alkyl as defined above.

The terms “aryl-C₁₋₆-alkyl” and “aryl-C₂₋₆-alkenyl” as used herein denotes aryl as defined above and C₁₋₆-alkyl and C₂₋₆-alkenyl, respectively, as defined above.

The term “acyl” as used herein denotes —(C═O)—C₁₋₆-alkyl wherein C₁₋₆-alkyl is as defined above.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent the substituents may be the same or different.

The term “treatment” as used herein means the prevention, management and care of a patient for the purpose of combating a disease, disorder or condition. The term is intended to include the prevention of the disease, delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The present invention relates to a new class of branched polymers, that are made up of a precise number of monomer building blocks that are oligomerised in any order either on solid support or in solution using suitable monomer protection and activation strategies.

An aspect of the invention provides a conjugate as described above, which is represented by the general formula
((branched polymer)-(L3)₀₋₁)ₓ-(peptide)
wherein the L3 is an linking moiety, and z is an integer ≧1 representing the number of branched polymers conjugated to the biologically active peptide.
Z is optionally 1, 2, 3, 4 or 5. In an aspect of the invention Z is 1 or 2;
L3 is as defined below for L1 and L2.

The monomer building blocks of the present invention are in general linear or branched bi-, tri- or tetrafurcated building blocks of the general structure A-L1-X-(L2-B)ₙ (general formula I) where X serves as attachment moiety for A-L1 as well as branching moiety for n number of L2-B, in which L1 and L2 both are linker moieties:

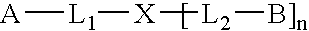

General formula I

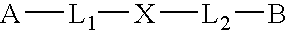

Ia
Linear
n = 1 in
general formula I

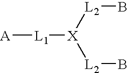

Ib
bifurcated
n = 2 in
general formula I

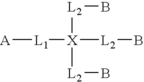

Ic
trifurcated
n = 3 in
general formula I

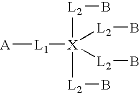

Id
tetrafurcated
n = 4 in
general formula I

A and B both are functional groups selected in such way, that they together under appropriate condition can form a covalent bond. The nature of the newly formed covalent bond depend upon the selection of A and B, and include but is not limited to: amide bonds, carbamate bonds, carbonate bonds, ester bonds, phosphate ester bonds, thiophosphate ester bonds, phosphoramidates, ether, and thioether bonds.

In an aspect of the invention A is selected from COOH, COOR, OCOOR, OP(NR₂)OR, O═P(OR)₂, S═P(OR)(OR′), S═P(SR)(OR′), S═P(SR)(SR′), COCl, COBr, OCOBr, CHO, Br, Cl, I, OTs, OMs, P(OR)₃, alkynes and azides, a p-nitrophenyl carbonate, succinimidyl carbonate, carbonylimidazole, carbonylchlorides, azlactone, cyclic imide thione, isocyanate or isothiocyanates, wherein R and R′ represents is C₁-alkyl, aryl or substituted aryl,

In an aspect of the invention A is a group of the formula: COOH, COOR, OCOOR, O═P(NR₂)OR, O═P(OR)₂, S═P(OR)(OR′), S═P(SR)(OR′), S═P(SR)(SR′), COCl, COBr, OCOCl, OCOBr, CHO, Br, Cl, I, OTs, OMs, alkynes and azides, wherein R and R′ represents is C₁₋₆-alkyl, aryl or substituted aryl,

In an aspect of the invention the moiety A of general formula 1, represent an activated moiety that can react with nucleophiles either on the peptide or of type B. Preferably A is selected from the group of:

Functional groups capable of reacting with amino groups such as
a) carbonates such as the p-nitrophenyl, or succinimidyl;
b) carbonyl imidazoles or carbonyl chlorides;
c) carboxylic acids that are activated in situ;
d) carbonyl halides, activated esters such as N-hydroxysuccinimide esters, N-hydroxybenzotriazole esters, esters of 1,2,3-benzotriazin-4(3H)-one
e) phosphoramidites and H-phosphonates
f) phosphortriesters or phosphordiesters activates in situ, or
g) isocyanates or isothiocyanates.

In an aspect of the invention B may be selected from NH₂, OH, N₃, NHR′, OR′, O—NH₂, alkynes, or any of the following

hydrazine derivatives —NH—NH₂,

hydrazine carboxylate derivatives —O—C(O)—NH—NH₂,

semicarbazide derivatives —NH—C(O)—NH—NH₂,

thiosemicarbazide derivatives —NH—C(S)—NH—NH₂,

carbonic acid dihydrazide derivatives —NHC(O)—NH—NH—C(O)—NH—NH₂,

carbazide derivatives —NH—NH—C(O)—NH—NH₂,

thiocarbazide derivatives —NH—NH—C(S)—NH—NH₂,

aryl hydrazine derivatives —NH—C(O)—C₆H₄—NH—NH₂, and

hydrazide derivatives —C(O)—NH—NH₂;

oxylamine derivatives, such as —C(O)—O—NH₂, —NH—C(O)—O—NH₂ and —NH—C(S)—O—NH₂

In an aspect of the invention R′ is a protection group including, but not limited to:

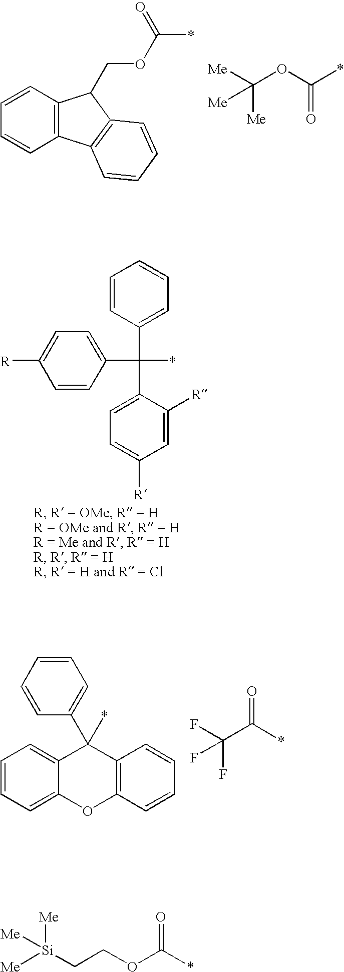

Other examples of appropriate protection groups are known to the skilled person, and suggestions can be found in Green & Wuts “Protection groups in organic synthesis”, 3.ed. Wiley-interscience.

In an aspect of the invention the moiety B of general formula 1, represent a protected nucleophile moiety that can react with electrophiles preferably of type A In an aspect of the invention B is selected from the group of:

a) Fmoc protected amino groups

b) free amino groups

c) azides, that can be reduced to amino groups

d) azides, that may participate together with alkynes to form triazoles

e) O-substituted hydroxylamines

f) hydroxyl groups

g) DMT, MMT or trityl-protected hydroxyl groups

In an aspect of the invention the covalent bond formed between A and B, depending on the respective choice of A and B, is amide bonds, oxime bonds, hydrazone bonds, semicarbozone bonds, carbonate bonds, carbamate bonds, ester bonds, phosphate ester bonds, thiophosphate ester bonds or phosphoramidates.

In an aspect of the invention the defintion of A and B may be interchanged to facilitate branched polymer assembly by the convergent approach as described below.

In an aspect of the invention X is either a linear (divalent organic radical) or a branched (multivalent branched organic radical) linker, preferably of hydrophilic nature. In an aspect of the invention it includes a multiply-functionalised alkyl group containing up to 18, and more preferably between 1-10 carbon atoms. Several heteroatoms, such as nitrogen, oxygen or sulfur may be included within the alkyl chain. The alkyl chain may also be branched at a carbon or a nitrogen atom. In an aspect of the invention, X is a single nitrogen atom

In an aspect of the invention X includes but is not limited to divalent organic radicals such as ethylene, arylene, propylene, ethyleneoxy,

or multivalent organic radicals such as propan-1,2,3-triyl, benzen-1,3,4,5-tetrayl, 1,1,1-nitrogentriyl or any of the groups below

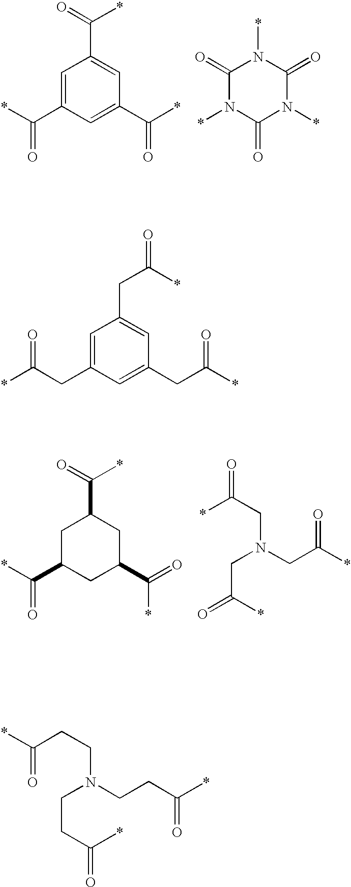

or a multivalent carbocyclic ring including, but is not limited to the following structures:

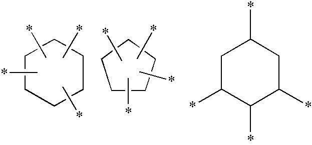

In an aspect of the invention X is

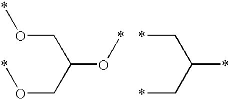

In an aspect of the invention X may be separated from A or B by linker L1 and L2, which preferably are of hydrophilic nature. Examples of such linkers include but is not limited to
1,2-ethandiyl, 1,3-propandiyl, 1,4-butandiyl, 1,5-pentandiyl, 1,6-hexandiyl, (CH₂CH₂O—)ₙ, where n is an integer between 0 and 10,
—(CR¹R²—CR³R⁴—O)ₙ—, where n is an integer between 0 and 10 and
R¹, R², R³ and R⁴ independently can be hydrogen or alkyl
((CH₂)ₘO)ₙ—, where m is 2, 3, 4, 5, 6, and n is an integer between 0 and 10, or succimidyl 4-malimidobutanoate or 1,6-bismalimidohexanes.

In an aspect of the invention X is symetrically.

In an aspect of the invention L1, L2 or both are valence bond.

In an aspect of the invention L1 and L2 are selected from water soluble organic divalent radicals. In an aspect of the invention either L1 or L2 or both are divalent organic radicals containing about 1 to 5 PEG (—CH₂CH₂O—) groups.

In an aspect of the invention L1 is -oxy- or -oxymethyl-, and L2 is (CH₂CH₂O—)₂:

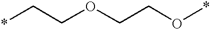

In an aspect of the invention A is a carboxyl group and B is a protected amino group which after deprotection may be coupled to a new monomer of same type via its carboxy group to form an amide.

In an aspect of the invention A is a phosphoramidite and B is a hydroxyl group suitable protected, which upon deprotection can be coupled to an other monomer of same type to form a phosphite triester which subsequently are oxidised to form a stable phosphate triester or thio phosphate triester.

In an aspect of the invention A is an reactive carbonate such as nitrophenyl carbonate, and B is an amino group, preferably in its protected form.

In an aspect of the invention A is an acyl halide such as COCl or COBr and B is an amino group, preferably in its protected form.

In an aspect of the invention A-L1-X-(L2-B)ₙ is

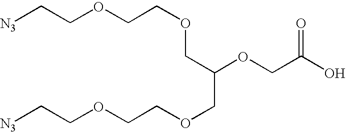

In an aspect of the invention A-L1-X-(L2-B)ₙ is

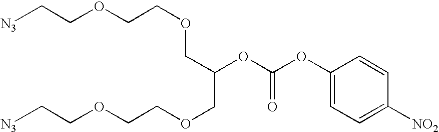

In an aspect of the invention A-L1-X-(L2-B)ᵣ is

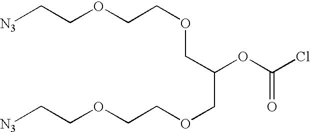

Branched polymers can in general be assembled from the monomers described above using one of two fundamentally different oligomerisation strategies called the divergent approach and the convergent approach.

In one aspect, the branched polymers are assembled by an iterative process of synthesis cycles, where each cycle use suitable activated, reactive bi-tri or multi furcated monomer building blocks, them self containing functional end groups—allowing for further elongation (i.e. polymer growth). The functional end groups usually needs to be protected in order to prevent self polymerisation and a deprotection step will in such cases be needed in order to generate a functional end group necessary for further elongation. One such cycle of adding a activated (reactive) monomer and subsequent deprotection, in the iterative process completes a generation. The divergent approach is illustrated in FIG. 4 using solution phase chemistry and in FIG. 3 using solid phase chemistry.

Convergent Assembly of Branched Polymers:

However, when higher generations materials are reached in such an itterative process, a high packing density of functional end groups will frequently appear, which prevent further regular growth leading to incomplete generations. In fact, with all systems in which growth requires the reaction of large numbers of surface functional groups, it is difficult to ensure that all will react at each growth step. This poses a significant problem in the synthesis of regular mono dispersed and highly organised branched structures since unreacted functional end groups may lead to failure sequences (truncation) or spurious reactivity at later stages of the stepwise growth sequence.

In one aspect of the invention, the branched polymer therefore is assembled by the convergent approach described in U.S. Pat. No. 5,041,516. The convergent approach to building macromolecules involves building the final molecule by beginning at its periphery, rather than at its core as in the divergent approach. This avoids problems, such as incomplete formation of covalent bonds, typically associated with the reaction at progressivly larger numbers of sites.

The convergent approach for assembly 2. generation branched polymer is illustrated in FIG. 1 and FIG. 2 using a specific example involving one of the monomer building blocks of the invention.

It is important to note, that the final branched polymer if desired may consist of different types of monomer building block in each of its generations. By using different monomers in each layer, branched polymers with tailored properties can be made. That way the overall properties of the polymer, and the polymer-peptide conjugate can be controlled.

In an aspect of the invention this provides the control the over all rigidity of the branched polymer. By choosing bifurcated monomers in the initial layer, followed by one or several layers of linear monomers, a polymer structure with a low number of branches and an overall floppy structure can be created. In an aspect of the invention the use of a highly branched monomer such as a tri- or tetrafurcated monomer repeatingly in each layer, while omitting any linear of low branched monomers, a hyper branched polymer with high density and overall compact structure can be obtained. Rigidity can also be controlled by the design of the particular monomer, for example by using a rigid core structure (X) or by using rigid linker moieties (L1, L2). In an aspect of the invention, adjustment of the rigity is then be obtained by using the rigid monomer in one or more specific layers intermixed with monomers of more flexible nature.

In an aspect of the invention the overall hydrophilic nature of the polymer is controllable. This is achieved by choosing monomers with more hydrophobic core structure (X) or more hydrophobic linker moieties (L1 & L2), in one or more of the dendritic layers.

In an aspect of the invention a different monomer in the outer layer of the branched polymer is used, which in the final peptide conjugate will be exposed to the surrounding environment. Some of the monomers described in this invention has protected amine functions as terminal end groups (B), which after a deprotection step, and under physiological conditions i.e. neutral physiological buffered pH around 7.4, will be protonated, causing the overall structure to be polycationically charged. Such polycationic structures has been proven to be toxic in animal studies and though they generally are rapidly cleared from the blood circulation system, they should be avoided in any pharmaceutical context. By selection of the suitable monomer used to create the final layer, polycationic structures can be avoided. One example as depicted in FIG. 5, uses a Me(Peg)2CH2COOH acid for capping the final layer of a dendritic structure, that otherwise would be terminated in amines.

In an aspect of the invention biopolymers is provided which imitates the natural occuring glycopeptides, which commonly has multiple anionic charged sialic acids as termination groups on the antenna structure of their N-glycans. Again according to the invention and by proper choice of the monomer used to create the final layer, such glycans can be imitated with respect to their poly anionic nature. One such example is depicted in FIG. 6, where the branched polymer is capped with succinic acid mono tert-butyl estes which upon deprotection with acids renders a polymer surface that are negatively charged under physiological conditions.

The assembly of monomers into polymers may be conducted either on solid support as described by N. J. Wells, A. Basso and M. Bradley in Biopolymers 47, 381-396 (1998) or in and appropriate organic solvent by classical solution phase chemistry as described by Frechet et al. in U.S. Pat. No. 5,041,516.

Thus in one aspect of the invention, the branched polymer is assembled on a solid support derivatised with a suitable linkage, in an iterative divergent process as described above and illustrated in FIG. 3. For monomers designed with Fmoc or Boc protected amino groups (B), and reactive functional acylating moieties (A), solid phase protocols useful for conventional peptide synthesis can conveniently be adapted. Applicably standard solid phase techniques such as those described in literature (see Fields, ed., Solid phase peptide synthesis, in Meth Enzymol 289) can be conducted either by use of suitable programmable instruments (e.g. ABI 430A) or similar home build machines, or manually using standard filtration techniques for separation and washing of support.

For monomers with e.g. DMT protected alcohol groups (B), and e.g. reactive phosphor amidites (A), solid phase equipment used for standard oligonucleotide synthesis such as Applied Biosystems Expidite 8909, and conditions such as those recently described by M. Dubber and J. M. J. Fréchet in Bioconjugate chem. 2003, 14, 239-246 can conveniently be applied. Solid phase synthesis of such phosphate diesters according to the conventional phosphoramidite methodology requiers that an intermediate phosphite triester is oxidised to a phosphate triester. This type of solid support oxidation is typically achived with iodine/water or peroxides such as but not limited to tert-butyl hydrogenperoxid and 3-chloroperbenzoic acid and requires that the monomers with or without protection resist oxidation condition. The phosphor amidite methodology also allows for convenient synthesis of thiophosphates by simple replacement of the iodine with elementary sulfur in pyridine or organic thiolation reagents such as 3H-1,2-benzodithiole-3-one-1,1-dioxide (see for example M. Dubber and J. M. J. Fréchet in Bioconjugate chem. 2003, 14, 239-246).

The resin attached branched polymer, when complete, can then be cleaved from the resin under suitable conditions. It is important, that the cleavable linker between the growing polymer and the solid support is selected in such way, that it will stay intact during the oligomerisation process of the individual monomers, including any deprotection steps, oxidation or reduction steps used in the individual synthesis cycle, but when desired under appropriate conditions can be cleaved leaving the final branched polymer intact. The skilled person will be able to make suitable choices of linker and support, as well as reaction conditions for the oligomerisation process, the deprotection process and optionally oxidation process, depends on the monomers in question.

In an aspect of the invention, the solid phase oligomerisation of branched monomers is conducted on an already existing solid phase tethered peptide, using either the deprotected N-terminal of the peptide as starting point, or any of the amino acid side chain residues, such as the ε-epsilon amino group of a lysin residue, the thiol group of a cystein or the hydroxy group of a serine, threonine or a tyrosine residue as starting point. It is also possible to use non-natural amino acids within a peptide sequence which carries unique chemical handles, as starting point for solid phase oligomerisation of the branched polymer.

Resins derivatised with appropriate functional groups, that allows for attachment of monomer units and later and act as cleavable moieties are commercial available (see f.ex the cataloge of Bachem and NovoBiochem).

In an aspect of the invention, the branched polymer is synthesised on a resin with a suitable linker, which upon cleavage generates a branched polymer product furnished with a functional group that directly can act as an attatchment group in a subsequent solution phase conjugation process to a peptide as described below, or alternatively, by appropriate chemical means can be converted into such an attachment group.

In an aspect of the invention the dendritic branched polymers of a certain size and compositions is synthesised using classical solution phase techniques.

In this aspect of the invention, the branched polymer is assembled in an appropriate solvent, by sequential addition of suitable activated monomers to the growing polymer. After each addition, a deprotection step may be needed before construction of the next generation can be initiated. It may be desirable to use excess of monomer in order to reach complete reactions. In one aspect of the invention, the removal of excess monomer takes advantages of the fact that hydrophilic polymers have low solubility in diethyl ether or similar types of solvents. The growing polymer can thus be precipitated leaving the excess of monomers, coupling reagents, biproducts etc. in solution. Phase separation can then be performed by simple decantation, of more preferably by centrifugation followed by decantation. Polymers can also be separated from biproducts by conventional chromatographic techniques on e.g. silica gel, or by the use of HPLC or MPLC systems under either normal or reverse phase conditions as described in P. R. Ashton et al. J. Org. Chem. 1998, 63, 3429-3437. Alternatively, the considerbly larger polymer can be separated from low molecular components, such as excess monomers and biproducts using size exclusion chromatography optionally in combination with dialysis as described in E. R. Gillies and J. M. J. Fréchet in J. Am. Chem. Soc. 2002, 124, 14137-14146.

In an aspect of the invention a convergent solution phase synthesis is used. In contrast to solid phase techniques, solution phase also makes it possible to use the convergent approach for assembly of branched polymers as described above and further reviewed in S. M. Grayson and J. M. J. Fréchet, Chem. Rev. 2001, 101, 3819-3867. In this approach it is desirable to initiate the synthesis with monomers, where the protected functional end groups (B) initially is converted into moieties that eventually will be present on the outer surface of the final branched polymer. Therefore the functional moiety (A) of general formula I in most cases will need suitable protection, that allows for stepwise chemical manipulation of the end groups (B). Protection groups for the functional moiety (A) depend on the actually functional group. For example, if A in general formula I is a carboxyl group, a tert-butyl ester derivate that can be removed by TFA would be an appropriate choice. Suitable protection groups are known to the skilled person, and other examples can be found in Green & Wuts “Protection groups in organic synthesis”, 3.ed. Wiley-interscience. The convergent assembly of branched polymers is illustrated in FIG. 1 and FIG. 2. In step (i) of FIG. 1, a tert-butyl ester functionallity (A) is prepared by reaction of a suitable precurser with t-butyl α-bromoacetate. In step (ii) the terminal end groups (B) is manipulated in such way that they allows for the acylation of step (iii), with a carboxylic acid that is converted into a acyl halid in step (iv). In step (v) the t-butyl ester functionality (A) is removed creating a end (B) capped monomer. This end capped monomer serves as starting material for preparing the second generation product in FIG. 2, where 2 equivalents is used in an acylation reaction with the product of step (ii) in FIG. 1. The product of this reaction is a new t-butyl ester, which after deprotection can re-enter in the initial step of FIG. 2 in a itterative manner creating higher generation materials.

To effect covalent attachment of the branced polymer molecule(s) to the peptide either in solution or on solid support, the branched polymer must be provided with a reactive handle, i.e. furnished with a reactive functional group examples of which includes carboxylic acids, primary amino groups, hydrazides, O-alkylated hydroxylamines, thiols, succinates, succinimidyl succinates, succimidyl proprionate, succimidyl carboxymethylate, hydrazides arylcarbonater and aryl carbamater such as nitrophenylcarbamates and nitrophenyl carbonates, chlorocarbonates, isothiocyanates, isocyanates, malemides, and activated esters such as:

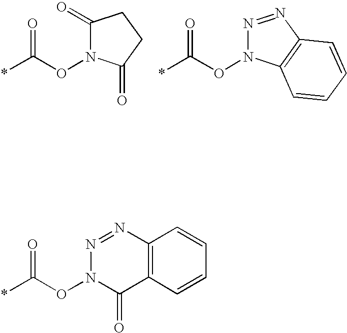

The conjugation of the branched polymer to the polypeptide is conducted by use of conventional methods, known to the skilled artisan. The skilled person will be aware that the activation method and/or conjugation chemistry (e.g. choice of reaction groups ect.) to be use depends on the attachment group(s) selected on the polypeptide (e.g. amino groups, hydroxyl groups, thiol groups ect.) and the branched polymer (e.g. succimidyl proprionates, nitrophenyl-carbonates, malimides, vinylsulfone, haloacetate ect.). In an aspect of the invention suitable attachment moieties on the branched polymer, such as those mentioned above, is created after the branched polymer has been assembled using conventional solution phase chemistry. Aspects of the invention illustrating different ways to create nucleophilic and electrophilic attachment moieties on a branched polymer containing a carboxylic acid group are listed in FIG. 7

In an aspect of the invention one or more of the activated branched polymers are attached to a biologically active polypeptides by standard chemical reactions. The conjugate is represented by the general formula II:
(((branched polymer)-(L3)₀₋₁)_{z}-(peptide) (formula II)

wherein (branched polymer) is a branched polymer consisting of monomers according to general formula I, L³ is an linking moiety essentially defined as for L¹ and L² of general formula I, (z) is an integer ≧1 representing the number of branched polymers conjugated to the biologically active polypeptide. The upper limit for (z) is determined by the number of available attachment sites on the polypeptide, and the preferred degree of branched polymer attachment.

The degree of conjugation is, as previously mentioned, modified by varying the reaction stoichiometry. More than one branched polymer conjugated to the polypeptide is obtained by reacting a stoichiometric excess of the activated polymer with the polypeptide.

The biologically active polypeptide is reacted with the activated branched polymers in an aqueous reaction medium which is optionally buffered, depending upon the pH requirements of the polypeptide. The optimum pH for the reaction is generally between about 6.5 and about 8 and preferably about 7.4 for most polypeptides.

The optimum reaction conditions for the polypeptide stability, reaction efficiency, etc. is within level of ordinary skill in the art. The preferred temperature range is between 4° C. and 37° C. The temperature of the reaction medium cannot exceed the temperature at which the polypeptide may denature or decompose. Preferably, the polypeptide be reacted with an excess of the activated branched polymer. Following the reaction, the conjugate is recovered and purified such as by diafiltration, column chromatography including size exclussion chromatotrapy, ion-exchange chromatograph, affinity chromatography, electrophoreses, or combinations thereof, or the like.

If suitable attachment groups such as amines, thiols or hydroxyl groups is not already present on the peptide, or modification of these interfere with the biological function of the peptide, suitable attachment groups is created on the native peptide by conventional genetic engineering, e.g. mutation on the DNA-level (e.g. coding codon replacement) of selected amino acids with amino acids allowing for post modificational attachment of polymers. The choice of which amino acid to mutate depend on the particular peptide. In general, it is desirable to select “allowed mutations” e.g. to select amino acids that will not affect the binding of the peptide to its natural ligands, or inhibit the peptides biological function such as enzymatic actions, substrate binding ect.

Mutation of DNA sequences using nonsense amber codons in conjunction with new genetically mutated tRNA synthethases selected to accept unnatural amino acids, is also a way to prepare peptides with unnatural amino acids under in vivo fermentation conditions (Wang, L. et al. PNAS U.S.A., 2003, 100, 56-61). Additionally, incorporation of novel amino acids with unique functional attachment groups, and post modification of these with glycomimetics is demonstrated (Liu, H.; Wang, L.; Brock, A.; Wong, C.-H.; Schultz, P. G.; J. Am. Chem. Soc.; (Communication); 2003; 125; 1702-1703). These gene products are suitable peptides according to the invention, as new non-natural chemoselective attachment moieties becomes available for modification with branched polymers.

In an aspect of the invention the peptide is assembled on solid phase and selected amino acids are substituted with amino acids with suitable side chains acting as attachment groups, using standard solid phase chemistry. Examples of such amino acid substitutions are by way of illustration: substitution of serine with cystein, substitution of phenylalanine with tyrosine or substitution of arginine with lysine. Alternatively, attachment groups are introduced by enzyme directed coupling in either the C- of N-terminal end of the peptide, with either suitable amino acids allowing for post modificational attachment of polymers, or small organic molecules serving the same purpose. Enzymes that supports this aspect of the invention include by way of illustration: carboxypeptidases, and proteases in reverse.

Natural peptides, obtained from eukaryote expression systems such as mammalian, insect or yeast cells, are frequently isolated in their glycosylated forms. The glycosyl moiety, also called the glycan moiety on such peptide, are them self polyalcohols which either directly can be used for conjugation purposes, or by appropriate conditions can be converted into suitable attachment moieties for conjugation.

Therefore, in an aspect of the invention, the branched polymer is conjugated using the glycan moieties present on the glycosylated peptide. The glycan's of interest are either O-linked glycanes, i.e. glycopeptides where the glycan is linked via the amino acids residues serine or threonine; or N-glycans where the glycan moiety is linked to asparagine residues of the peptide.

In an aspect of the invention modification of the glycan in necessary, in order to subsequently attach the branched polymer. In an aspect of the invention, the N-glycans present on a peptide is oxidised enzymatically using galactose oxidase as described in Fu, Q. & Gowda, D. C. Bioconjugate Chem. 2001, 12, 271-279, thereby creating free aldehyde functionalities that function as attachment moieties for a branched polymer made according to the invention. In this particular aspect, it the sialylated peptide is optionally treated with sialidase prior to the galactose oxidase treatment, in order to expose free galactose residues on the surface of the peptide.

Thus in an aspect of the invention, a peptide is treated enzymatically with sialidases, followed by galactose oxidase, to create reactive aldehyde functionalities on the surface of the peptide. These are then reacted with a branched polymer, containing one of either an oxime, hydrazine or hydrazide handle such as those prepared in FIG. 7, thereby completing the conjugation process. In an aspect of the invention the water soluble polymer is covalently attached to a monosaccharide which is converted into an activated substrate for a particular glycosyl transferase as recently described in WO03/031464 (Neose).

N- and O-glycanes are directly converted into aldehyde functionalities by chemical means. Thus in an aspect of the invention, the glycosylated peptide is submitted to periodate treatment under neutral conditions, thereby generating reactive aldehyde functionalities.

Thus in a first aspect, the present invention provides a method for producing a conjugate of a glycopeptide comprising a glycopeptide having at least one terminal galactose derivative and a protractor group covalently bonded thereto,

the method comprising the steps of:
(a) contacting a glycopeptide having at least one terminal galactose derivative with galactose oxidase to create a glycoprotein comprising an oxidised terminal galactose derivative having a reactive aldehyde functionality;
(b) contacting the glycopeptide product produced in step (a) with a reactant X capable of reacting with an aldehyde group; wherein X is a protractor group to create a conjugate represented by the formula (glycopeptide)-(protractor group).

In a second aspect, the present invention provides a method for producing a conjugate of a glycopeptide having increased in vivo plasma half-life compared to the non-conjugated glycopeptide, the conjugate comprising a glycopeptide having at least one terminal galactose or derivative thereof, and a protractor group covalently bonded to the thereto through a linking moiety;

the method comprising the steps of:
(a) contacting a glycopeptide having at least one terminal galactose or derivative thereof with galactose oxidase to create a glycopeptide comprising an oxidised terminal galactose or derivative thereof having a reactive aldehyde functionality;
(b) contacting the glycopeptide product produced in step (a) with a reactant X capable of reacting with an aldehyde group, wherein X is a linking moiety comprising a second reactive, optionally protected, group to create a conjugate of the glycopeptide and the linking moiety;
(c) contacting the product of step (b) with a protractor group capable of reacting with the second reactive group of the linking moiety to create a conjugate represented by the formula (glycopeptide)-(Linking moiety)-(protractor group).

A preferred glycopeptide for the conjugation step is a glycopeptide which has been treated with sialidase to remove sufficient sialic acid to expose at least one galactose residue and which has been further treated, e.g., with galactose oxidase and horseradish peroxidase to produce a free reactive aldehyde functionality.

A preferred reaction sequence is depicted below, using a reactant X capable of reacting with an aldehyde group:

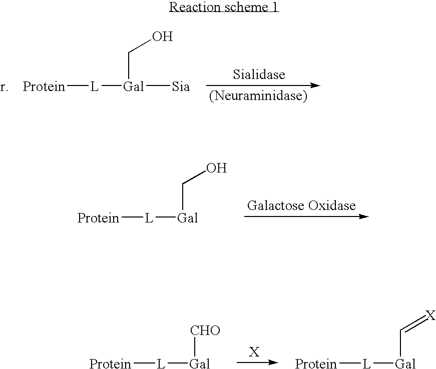

where Sia denotes a sialic acid linked to a galactose or galactose derivative (Gal) in either alpha-2,3-, or alpha-2,6-configuration.

In one aspect the Gal-OH represent galactose in which case,

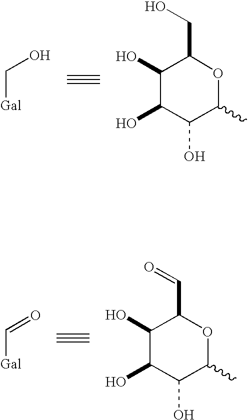

In one aspect Gal-OH represent the galactose derivative N-acetyl galactosamine and the galactose oxidase oxidizes the acetylated galactosamine residues in which case,

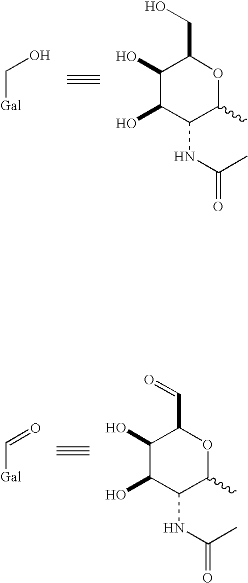

X is any type of molecule containing a chemical functionality that can react covalently with an aldehyde to form a C-6 modified galactose or N-acetyl galactosamine residue (such as, e.g., a nucleophile agent).

L is a divalent organic radical linker which may be any organic di-radical including those containing one or more carbohydrate moiety(-ies) consisting of natural monosaccharide(s), such as fucose, mannose, N-acetyl glycosamine, xylose, and arabinose, interlinked in any order and with any number of branches. L may also be a valence bond.

The chemical conjugation may be performed in a number of ways depending on the particular reactant X involved.

In an aspect, X is a nucleophile, which can form a covalent linkage upon dehydration.

Non-limiting examples for illustration include hydroxylamines, O-alkylated hydroxylamines, amines, stabilised carbanions, stabilised enolates, hydrazides, alkyl hydrazides, hydrazines, acyl hydrazines, α-mercaptoacylhydrazides etc. Other aspects includes ring forming (e.g. thiazolidine forming) nucleophiles such as, e.g., thioethanamines, cystein or cystein derivatives.

In some cases (vide infra) the product of the reaction may be further reacted with a reducing agent (a reductant) to form reduced products as indicated below:

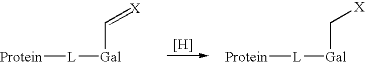

In such cases, preferred, and non-limiting, examples of reducing agents (reductants) include sodium cyanoborohydride, pyridine borane, and sodium borohydride, and preferred examples of x includes hydrazides, primary and secondary amines.

In general, O-alkylated hydroxylamine derivatives, when reacted with aldehydes form stable oxime derivatives spontaneously:

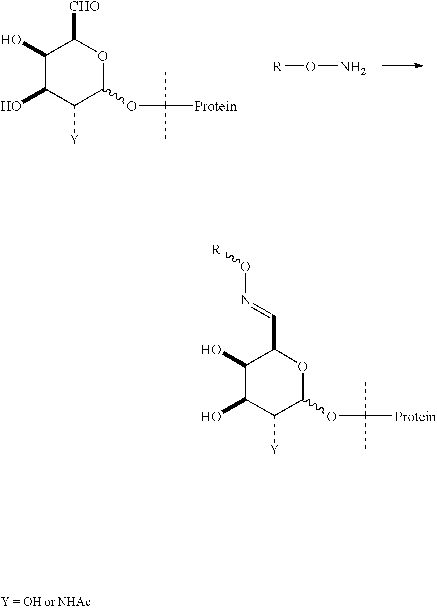

Though more reactive, and in some cases directly destructive to the peptide in question, alkyl hydrazines also react efficiently with aldehydes to produce hydrazones. Hydrazones are stable in aqueous solution and may therefore be considered as an alternative to hydroxylamines for derivatisation:

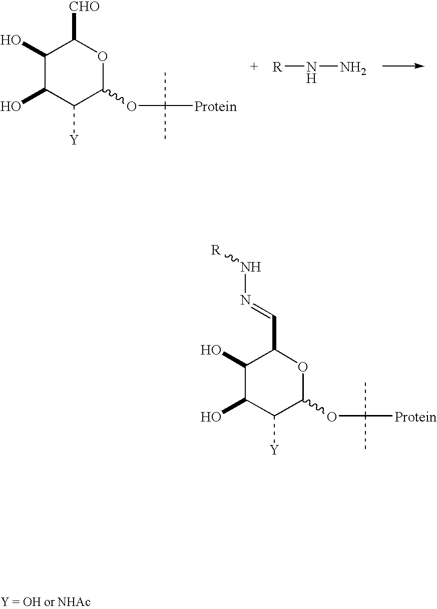

Hydrazides on the other hand, also react spontaneously with aldehydes, but the acyl hydrazone product is less stable in aqueous solution. When using hydrazide derivatived ligands, the resultant hydrazone is therefore frequently reduced to N-alkyl hydrazide using mild reduction reagents such as sodium cyanoborohydride or pyridine borane. See for example Butler T. et al. Chembiochem. 2001, 2(12) 884-894.

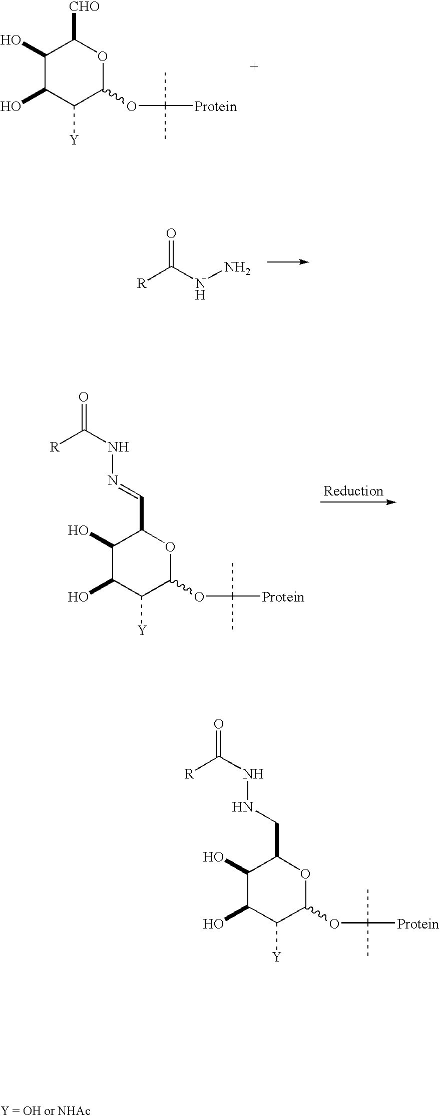

Formation of Schiff-bases between amines and aldehydes offers another type of chemical conjugation methodology. As in the case of hydrazides, a mild reduction of the imine to produce amines is frequently required in order to obtain a stable conjugate.

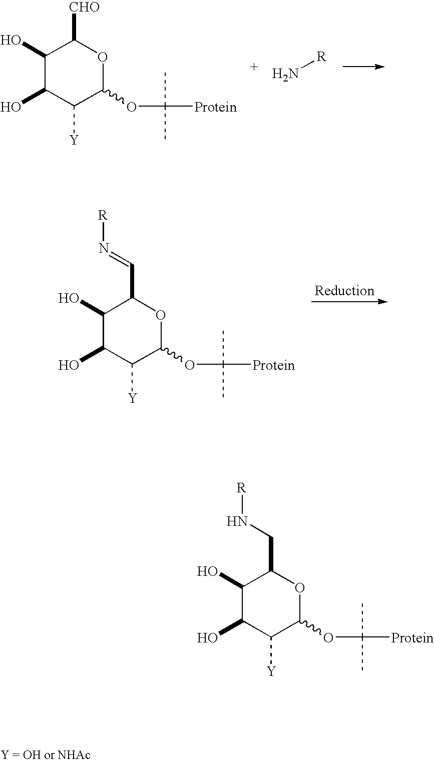

Although mild reduction reagents are known some difficulties in avoiding reduction of sulphide-sulphide (SS) bridges in the peptide can be foreseen. In such cases, a chemically conjugation principle that avoid reducing agents is preferred.

C6-oxidised galactose residues also react efficiently with amino thiols such as cystein or cystein derivatives or aminoethane thiol to produce thiazolidines as depicted below:

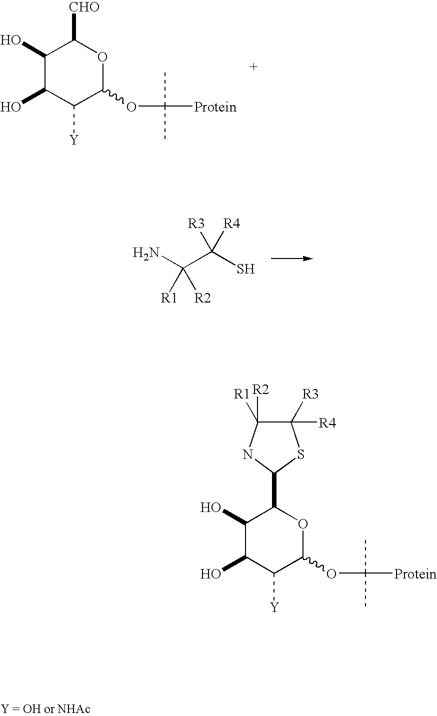

A similar type of modification that also leads to cyclic products involves α-mercaptoacylhydrazides:

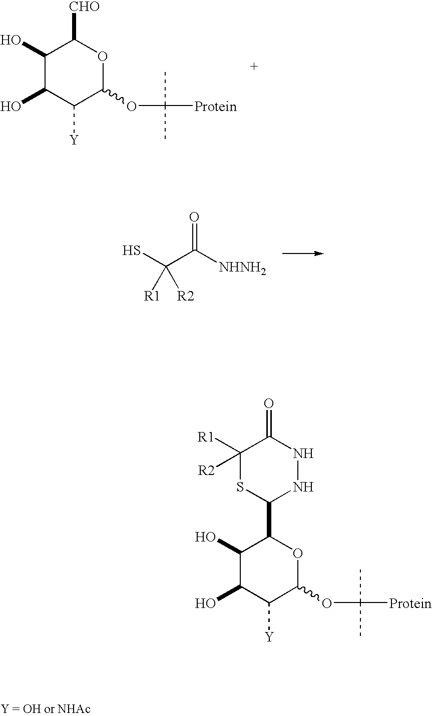

C6-oxidised galactose residues can also react with carbanionic organophosphorus reagents in a Horner-Wadsworth-Emmons reaction. The reaction forms an alkene as depicted below. The strength of the nucleophile can be varied by employing different organophosphorus reagents, like those employed in the Wittig reaction.

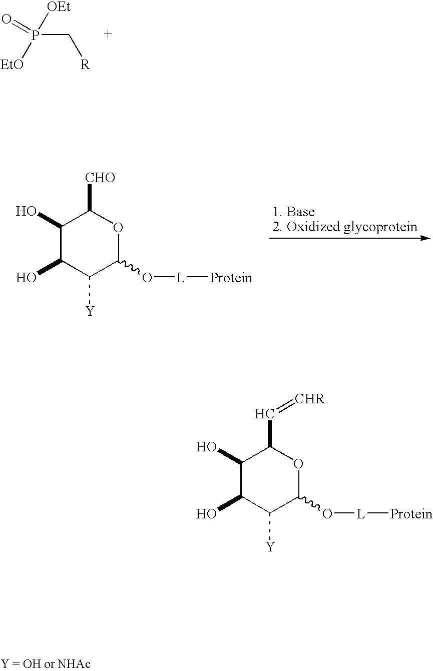

C6-oxidised galactose residues can also react with carbanion nucleophiles. An example of this could be an aldol type reaction as illustrated below. The Z′ and Z″ groups represent electron withdrawing groups, such as COOEt, CN, NO₂ (see March, Advanced Organic Chemistry, 3rd edition, John Wiley & Sons, N.Y. 1985), which increase the acidity of the methylene protons. In the invention, one or both of the Z groups would also be connected to an R group (protractor), which could improve the properties of the glycopeptide.

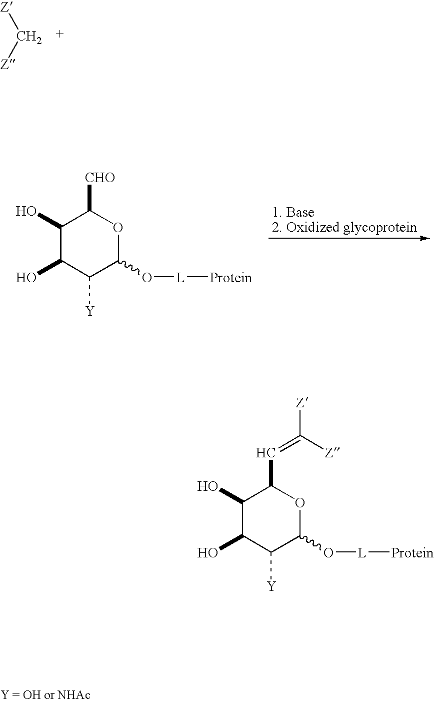

The above listed examples for modifying galactose oxidised in the C6 positions serves as non-limiting examples of the present invention. Other nucleophiles and chemical procedures for modifying aldehyde functions such as those present on C6-oxidised galactose are known to the skilled person (see March, Advanced Organic Chemistry, 3rd edition, John Wiley & Sons, N.Y. 1985).

Linker Molecules

Modification of the oxidised (asialo) glycopeptide, may also proceed in more than one step, before reaching to the final product. Thus, in one aspect the C6 oxidised galactose residue is initially reacted with a linker molecule possessing specificity for the aldehyde moiety. The linker molecule, itself containing an additional chemical handle (bifunctional), is then reacted further by attaching another molecule (e.g. a protractor moiety) to give the final product:
Glycopeptide-CHO→Glycopeptide-Linker-X→glycopeptide-Linker-R

Suitable bifunctional linkers are well known to the skilled person, or can easilly be conceived. Examples include, but are not limited to bifunctional linkeres containing hydroxylamine-, amine-, or hydrazied in combination with malimides, succimidyl ester, thiols hydroxylamines, amines, hydrazides or the like.

Although written as stepwise reactions in reaction scheme 1 above, it may in some cases be preferable to add the nucleophile directly into the reaction mixture when performing the oxidation using the galactose oxidase-catalase or the galactose oxidase horseradish peroxidase enzyme couple. Such one-pot conditions can prevent any intermolecular peptide reactions of the aldehyde functionalities on one peptide with the amino groups (e.g. epsilon amines in lysine residues) on the other. Intra and intermolecular Schiff base (imine) formation between peptides can lead to incomplete reaction with the nucleophile, or precipitation of the peptide in question. One pot conditions also prevent any possible over-oxidation mediated by galactose oxidase, as the aldehyde functionality instantly can react with the nucleophile present in the reaction media. The concentration ratio of nucleophile to peptide may depend on the peptide in question and the type of nucleophile (e.g. hydroxylamine, hydrazide, amine, etc.) selected for conjugation. Optimal conditions may be found by experiments, e.g. perform variation in the concentration ratio of nucleophile to peptide, perform variation in the overall concentration of peptide in solution, etc.

While the galactose or N-acetylgalactosamine residue(s) is/are generally exposed after treatment with sialadase, the invention can be used to covalently bind a protractor moiety to any terminal galactose moiety. One example could be the addition of terminal galactose residues to a glycan by the use of galactosyl transferases, and such terminal galactose residues could be modified by the technology described by the invention.

In an aspect of the invention the branched polymer(s) are coupled to the peptide through a linker. Suitable linkers are well known to the person skilled in the art. Examples include but is not limited to N-(4-acetylphenyl)malimide, succimidyl ester activatede malimido derivatives such as commercial available succimidyl 4-malimidobutanoate, 1,6-bismalimidohexanes. Other linkers include divalent alkyl derivatives optionally containing heteroatoms. Examples include the following:
1,2-ethandiyl, 1,3-propandiyl, 1,4-butandiyl, 1,5-pentandiyl, 1,6-hexandiyl, (CH₂CH₂O—)ₙ, where n is an integer between 0 and 10,
—(CR¹R²—CR³R⁴—O)ₙ—, where n is an integer between 0 and 10 and
R¹, R², R³ and R⁴ independently can be hydrogen or alkyl
((CH₂)ₘO)ₙ—, where m is 2, 3, 4, 5, 6, and n is an integer between 0 and 10,

It will be understood, that depending on the circumstances, e.g. the amino acid sequence of the peptide, its secondary and tertiary structure and the accessiability of attachment group(s) on the peptide, the nature of the activated branched polymer attached and the specific conjugation conditions, including the molar ratio of or branched polymer to peptide, variating degrees of polymer derivatised peptide is obtained, with a higher degree of polymer derivatised peptide obtained with a higher molar ratio of activated polymer to peptide. The polymer derivatised peptide (the conjugate) resulting from such process will however normally comprise a stochastic distribution of peptide conjugates having slightly different degree of polymer modifications.

In an aspect of the invention the method of conjugation is based upon standard chemistry, which is performed in the following manner. The branched polymer has an aminooxyacetyl group attached during synthesis, for example by acylation of diaminoalkyl linked aminooxyacetic acid as depicted in FIG. 7. The peptide has a terminal serine or threonine residue, which is oxidised to a glyoxylyl group under mild conditions with periodate according to Rose, J. Am. Chem. Soc. 1994, 116, 30-33 and European Patent 0243929. The aminooxy component of the branched polymer and the aldehyde component of the peptide are mixed in approximately equal proportions at a concentration of 1-10 mM in aqueous solution at mildly acid pH (2 to 5) at room temperature and the conjugation reaction (in this case oximation) followed by reversed phase high pressure liquid chromatography (HPLC) and electrospray ionisation mass spectrometry (ES-MS). The reaction speed depends on concentrations, pH and steric factors but is normally at equilibrium within a few hours, and the equilibrium is greatly in favour of conjugate (Rose, et al., Biacanjugate Chemistry 1996, 7,552-556). A slight excess (up to five fold) of one component forces the conjugation reaction towards completion. Products are isolated and characterised as previously described for oximes. Peptides (e.g. insulin) are purified for example by reversed phase HPLC (Rose, J Am. Chem. Soc., supra and Rose, et al., Bioconjugate Chemistry, supra) where as larger peptides (e.g. antibodies and their fragments) are optionally purified by ion-exchange chromatography, or by gel filtration techniques as for the trioxime described by Werlen, et al., Cancer Research 1996, 56,809-815.

In an aspect of the invention the method of conjugation is performed in the following manner. The branched polymer is synthesised on the Sasrin, or Wang resin (Bachem) as depicted in FIG. 3. Using the procedure recommended by the resin manufacturer (Bachem), the branched polymer is cleaved from the resin by repeated treatment with TFA in dichloromethane and the solution of cleaved polymer is neutralised with pyridine in methanol. After evaporation of solvents at room temperature (no heat is applied) and purification of the cleaved polymer as if it were a peptide, the carboxyl group which was connected to the resin is activated (e.g. with HBTU, TSTU or HATU) and coupled to a nucleophilic group (such as an amino group, i.e. an epsilon amino group on the side chain of lysin) on the peptide by standard techniques of peptide chemistry. If desired, the modified target molecule or material can be purified from the reaction mixture by one of numerous purification methods that are well known to those of ordinary skill in the art such as size exclusion chromatography, hydrophobic interaction chromatography, ion exchange chromatography, preparative isoelectric focusing, etc. General methods and principles for macromolecule purification, particularly peptide purification, can be found, for example, in “Protein Purification: Principles and Practice” by Seeres, 2nd ed., Springer-Verlag, New York, N.Y., (1987) which is incorporated herein by reference.

The peptides conjugated with the branched polymers are described as “biologically active”. The term, however, is not limited to physiological or pharmacological activities. For example, some inventive polymer conjugates containing peptides such as immunoglobulin, enzymes with proteolytical activities and the like are also useful as laboratory diagnostics, i.e. for in vivo studies ect. A key feature of all of the conjugates is that at least same activity associated with the unmodified bio-active peptide is maintained, unless a diminished activity is favourable as described in the present invention, or if a diminished activity could be accepted due to other properties of the conjugate obtained.

The conjugates thus are biologically active and have numerous therapeutic applications. Humans in need of treatment which includes a biologically active peptide can be treated by administering an effective amount of a branched polymer conjugate containing the desired bioactive peptide. For example, humans in need of enzyme replacement therapy or blood factors can be given branched polymer conjugates containing the desired peptide.

Biologically active peptides of interest of the present invention include, but are not limited to, peptides and enzymes. Enzymes of interest include carbohydrate-specific enzymes, proteolytic enzymes, oxidoreductases, transferases, hydrolases, lyases, isomerases and ligasese, without being limited to particular enzymes, examples of enzymes of interest include asparaginase, arginase, arginine deaminase, adenosine deaminase, superoxide dismutase, endotoxinases, cataiases, chymotrypsin, lipases, uricases, adenosine diphosphatase, tyrasinases and bilirubin oxidase. Carbohydrate-specific enzymes of interest include glucose oxidases, glycosidases, galactosidases, glycocerebrosidases, glucouronidases, etc.

Peptides of interest include, but are not limited to, hemoglobin, serum peptides such as blood factors including Factors VII, VIII, and IX; immunoglobulins, cytokines such as interleukins, α-, β- and γ-interferons, colony stimulating factors including granulocyte colony stimulating factors, platelet derived growth factors and phospholipase-activating peptide (PLAP). Other peptides of general biological and therapeutic interest include insulin, glucagon, glucagon-like peptide 1 (GLP1), glucagon-like peptide 2 (GLP2); oxyntomodulin (glucagon 1-37), human growth factor, plant proteins such as lectins and ricins, tumor necrosis factors and related alleles, soluble forms of tumor necrosis factor receptors, growth factors such as tissue growth factors, such as TGFα's or TGFβ's and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and the like. Immunoglobulins of interest include IgG, IgE, IgM, IgA, IgD and fragments thereof.

In an aspect of the invention the peptide is aprotinin, tissue factor pathway inhibitor or other protease inhibitors, insulin, insulin precursors or insulin analogues, human or bovine growth hormone, interleukin, glucagon, GLP-1, GLP-2, IGF-I, IGF-II, tissue plasminogen activator, transforming growth factor α or β, platelet-derived growth factor, GRF (growth hormone releasing factor), immunoglubolines, EPO, TPA, protein C, blood coagulation factors such as FVII, FVIII, FIV and FXIII, exendin-3, exentidin-4, and enzymes or functional analogues thereof. In the present context, the term “functional analogue” is meant to indicate a peptide with a similar function as the native peptide. The peptide may be structurally similar to the native peptide and may be derived from the native peptide by addition of one or more amino acids to either or both the C- and N-terminal end of the native peptide, substitution of one or more amino acids at one or a number of different sites in the native amino acid sequence, deletion of one or more amino acids at either or both ends of the native peptide or at one or several sites in the amino acid sequence, or insertion of one or more amino acids at one or more sites in the native amino acid sequence. Furthermore the peptide may be acylated in one or more positions, vide WO 98/08871 which discloses acylation of GLP-1 and analogues thereof and in WO 98/08872 which discloses acylation of GLP-2 and analogues thereof. An example of an acylated GLP-1 derivative is Lys²⁶(N^{ε}-tetradecanoyl)-GLP-1₍₇₋₃₇₎ which is GLP-1₍₇₋₃₇₎ wherein the ε-amino group of the Lys residue in position 26 has been tetradecanoylated.

An insulin analogue is an insulin molecule having one or more mutations, substitutions, deletions and or additions of the A and/or B amino acid chains relative to the human insulin molecule. The insulin analogues are preferably such wherein one or more of the naturally occurring amino acid residues, preferably one, two, or three of them, have been substituted by another codable amino acid residue. Thus position 28 of the B chain may be modified from the natural Pro residue to one of Asp, Lys, or Ile. In another aspect Lys at position B29 is modified to Pro; also, Asn at position A21 may be modified to Ala, Gln, Glu, Gly, His, Ile, Leu, Met, Ser, Thr, Trp, Tyr or Val, in particular to Gly, Ala, Ser, or Thr and preferably to Gly. Furthermore, Asn at position B3 may be modified to Lys. Further examples of insulin analogues are des(B30) human insulin, insulin analogues wherein PheB1 has been deleted; insulin analogues wherein the A-chain and/or the B-chain have an N-terminal extension and insulin analogues wherein the A-chain and/or the B-chain have a C-terminal extension. Thus one or two Arg may be added to position B1. Also, precursors or intermediates for other peptides may be treated by the method of the invention. An example of such a precursor is an insulin precursor which comprises the amino acid sequence B(1-29) AlaAlaLys-A(1-21) wherein A(1-21) is the A chain of human insulin and B(1-29) is the B chain of human insulin in which Thr(B30) is missing. Finally, the insulin molecule may be acylated in one or more positions, such as in the B29 position of human insulin or desB30 human insulin. Examples of acylated insulins are N^{εB29}-tetradecanoyl Gln^{B3} des(B30) human insulin, N^{εB29}-tridecanoyl human insulin, N^{εB29}-tetradecanoyl human insulin, N^{εB29}-decanoyl human insulin, and N^{εB29}-dodecanoyl human insulin.

Some peptides such as the interleukins, interferons and colony stimulating factors also exist in non-glycosylated form, usually as a result of using recombinant techniques. The non-glycosylated versions are also among the biologically active peptides of the present invention. The biologically active peptides of the present invention also include any fragment of a peptide demonstrating in vivo bioactivity. This includes amino acid sequences, antibody fragments, single chain binding antigens, see, for example U.S. Pat. No. 4,946,778, binding molecules including fusions of antibodies or fragments, polyclonal antibodies, monoclonal antibodies, and catalytic antibodies.

The peptides or fragments thereof can be prepared or isolated by using techniques known to those of ordinary skill in the art such as tissue culture, extraction from animal sources, or by recombinant DNA methodologies. Transgenic sources of the peptides are also contemplated. Such materials are obtained form transgenic animals, i.e., mice, pigs, cows, etc., wherein the peptides expressed in milk, blood or tissues. Transgenic insects and baculovirus expression systems are also contemplated as sources. Moreover, mutant versions, of peptides, such as mutant TNF's and/or mutant interferons are also within the scope of the invention. Other peptides of interest are allergen peptides such as ragweed, Antigen E, honeybee venom, mite allergen, and the like.

The foregoing is illustrative of the biologically active peptides which are suitable for conjugation with the polymers of the invention. It is to be understood that those biologically active materials not specifically mentioned but having suitable peptides are also intended and are within the scope of the present invention.

In an aspect of the invention water soluble polymers of the subject invention are provides. These are important as agents for enhancing the properties of the peptides. For example coupling water soluble polymers, to peptides to increased solubility of the modified peptide as compared with the native peptide at physiological pH when the native peptide is insoluble or only partially soluble at physiological pH. The attachment of branched polymers to peptides provides conjugates which provides decreased immune response compared to the immune response generated by the native peptide, or an increased pharmacokinetic profile, an increased shelf-life, and an increased biological half-life. The invention provides peptides which are modified by the attachment of the hydrophilic water soluble branced polymers of the invention, without substantially reducing or interfering with the biologic activity of the non modified peptide.

The invention provides peptides, modified by the structural well defined polymers of the invention are essentially homogeneous compounds, wherein the number of generations of the branched polymer is well-defined.

The invention provides conjugates which has maintained the biological activity of the non conjugated peptide. In an aspect of the invention the conjugated peptide has improved characteristics compared to the non-conjugated peptide.

In an aspect of the invention the branched polymers made according to the invention, when conjugated to certain parts of a polypeptide, reduces the bioavailability, the potency, the efficacy or the activity of a particular polypeptide. Such reduction can be desirable in drug delivery systems based on the sustain release principle. In an aspect of the invention, a sustain release principle in which the branched polymer is used in connection with a linker that can be cleaved under physiological conditions, thereby releasing the bio-active polypeptide slowly from the branched polymer, is contemplated within the invention. In this case, the polypeptide will not be biological active before the branched polymer is removed. In a specific aspect, the cleavable linker is a small peptide, that can function as a substrate for e.g. proteases present in the blood serum.

In an aspect of the invention a biological active polypeptide is conjugated via a protease labile linker to a branched polymer made according to the invention.

In an aspect of the invention biological active polypeptides are conjugated via protease labile linkers to a branched polymer prepared according to the invention.

It will be understood that the polymer conjugation is designed so as to produce the optimal molecule with respect to the number of polymer molecules attached, the size and composition of such molecules (e.g. number of generations and particular monomer used in each generation), and the attachment site(s) on the peptide derivative. The molecular weight of the polymer to be used may e.g., be chosen on the basis of the desired effect to be achieved. The particular molecular weight of the branched polymer to be used may e.g. be chosen on the basis of the desired effect to be achieved. For instance, if the primary purpose of the conjugate is to achieve a conjugate having a high molecular weight (e.g., to reduce renal clearence) it is usually desirable to conjugate as few high molecular branched polymer molecules as possible to obtain the desirable molecular weight. In other cases, protection against specific or unspecific proteolytical cleavage or shielding of an immunogenic epitope on the peptide can be desirable, and a branched polymer with a specific low molecular weight may be the optimal choice.

Thus, by the methods of this invention polymer derivatised peptides (conjugates) with a fine-tuned predefined mass is obtained.

In an aspect, a branched polymer synthesised according to the invention, with a specific structure and a well defined mass, is conjugated to FVIIa to produce a product with a substantial improved pharmacodynamical and pharmacokinetical profile in human blood and serum.

In another aspect, a branched polymer synthesised according to the invention is conjugated to GLP1 or GLP2. In an aspect of this, it prevents DPPIV mediated proteolytical cleavage.

In still another aspect of the invention, a branched polymer prepared according to the invention is conjugated to insulin. In an aspect of the invention this produces a conjugate with increased pulmonal bioavailability.

In still another aspect, a branched polymer prepared according to the invention is used to shield neoepitopes on refolded peptide drugs against potential immunogenicity, by conjugating the branched polymer to an attachment group on the refolded peptide.

In a related aspect, a branched polymer according to the invention is used to shield immunogenic epitopes on biopharmaceutical peptide obtained from non-human sources.

In another aspect, a branched polymer is used to substantially increase the molecular weight of a small peptide. In an aspect this reduces the renal clearence.

In yet another aspect, a branched water soluble polymer made according to the invention is conjugated to a peptide, that in its unmodified state and under physiological conditions has a low solubility.

In an aspect, the in vivo half life of certain peptide conjugates of the invention is improved by more than 10%. In an aspect, the in vivo half life of certain peptide conjugates is improved by more than 25%. In an aspect, the in vivo half life of certain peptide conjugates is improved by more than 50%. In an aspect, the in vivo half life of certain peptide conjugates is improved by more than 75%. In an aspect, the in vivo half life of certain peptide conjugates is improved by more than 100%. In another aspect, the in-vivo half life of a certain peptide is increased 250% upon conjugation of a branched polymer.

In an aspect, the functional in vivo half life of certain peptide conjugates of the invention is improved by more than 10%. In an aspect, the functional in vivo half life of certain peptide conjugates is improved by more than 25%. In an aspect, the functional in vivo half life of certain peptide conjugates is improved by more than 50%. In an aspect, the functional in vivo half life of certain peptide conjugates is improved by more than 75%. In an aspect, the functional in vivo half life of certain peptide conjugates is improved by more than 100%. In another aspect, the functional half life of a certain peptide is increased 250% upon conjugation of a branched polymer.

Generally, the stability of peptides in solution is very poor. Therefore, in one aspect of the invention, well defined water soluble branched polymers as described herein can conjugate peptides and stabilize the peptide by minimizing structural transformations such as refolding and maintain peptide activity.

In a related aspect, the shelf-half life of a peptide is improved upon conjugation to a branched polymer of the invention.

Pharmaceutical Compositions

The present invention also relates to pharmaceutical compositions comprising, as an active ingredient, at least one of the compounds of the present invention or a pharmaceutically acceptable salt thereof and, usually, such compositions also contain a pharmaceutically acceptable carrier, surfactant or diluent. The pharmaceutical compositions of the invention can also comprise combinations with other compounds as described.

Pharmaceutical compositions comprising a compound of the present invention may be prepared by conventional techniques, e.g. as described in Remington: The Science and Practise of Pharmacy, 19^{th} Ed., 1995. The compositions may appear in conventional forms, for example capsules, tablets, aerosols, solutions or suspensions.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen. The route of administration may be any route, which effectively transports the active compound to the appropriate or desired site of action.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, such as from about 0.01 to about 50 mg/kg body weight per day, for example from about 0.05 to about 10 mg/kg body weight per day administered in one or more dosages such as 1 to 3 dosages. The exact dosage will depend upon the nature of the peptide, together with the combination agent chosen, the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, for example from about 0.1 to about 500 mg, such as from about 0.5 mg to about 200 mg.

For parenteral routes such as intravenous, intrathecal, intramuscular and similar administration, typically doses are in the order of about half the dose employed for oral administration.

Salts of polypeptides or small molecules are especially relevant when the compounds is in solid or crystalline form

For parenteral administration, solutions of the compounds of the invention, optionally together with the combination agent in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax.

The pharmaceutical compositions formed by combining a compound of the invention and the pharmaceutically acceptable carriers are then readily administered in a variety of dosage forms suitable for the disclosed routes of administration. The formulations may conveniently be presented in unit dosage form by methods known in the art of pharmacy.

For nasal administration, the preparation may contain a compound of the invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Formulations of a compound of the invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain a compound of the invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions of a compound of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservatives and flavouring and colouring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

A compound of the invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds of the invention may form solvates with water or common organic solvents. Such solvates are also encompassed within the scope of the invention.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A compound of the invention may be administered to a mammal, especially a human, in need of such treatment. Such mammals include also animals, both domestic animals, e.g. household pets, and non-domestic animals such as wildlife.

Pharmaceutical compositions containing a compound according to the invention may be administered one or more times per day or week, conveniently administered at mealtimes. An effective amount of such a pharmaceutical composition is the amount that provides a clinically significant effect. Such amounts will depend, in part, on the particular condition to be treated, age, weight, and general health of the patient, and other factors evident to those skilled in the art.

In one aspect the invention relates to a pharmaceutical composition of the invention comprising an amount of a compound of the invention effective to promote angiogenesis.

In another aspect the invention relates to a pharmaceutical composition of the invention comprising an amount of a compound of the invention effective to inhibit angiogenesis.

A convenient daily dosage can be in the range from 1-1000 microgram/kg/day. In another aspect from 5-500 microgram/kg/day. If the body weight of the subject changes during treatment, the dose of the compound might have to be adjusted accordingly.

A compound of the invention optionally together with the combination agent for use in treating disease or disorders according to the present invention may be administered alone or in combination with pharmaceutically acceptable carriers or excipients, in either single or multiple doses. The formulation of the combination may be as one dose unit combining the compounds, or they may be formulated as seperate doses. The pharmaceutical compositions comprising a compound of the invention optionally together with the combination agent for use in treating angiogenesis according to the present invention may be formulated with pharmaceutically acceptable carriers or diluents as well as any other known adjuvants and excipients in accordance with conventional techniques such as those disclosed above.

Another object of the present invention is to provide a pharmaceutical formulation comprising a compound according to the present invention which is present in a concentration from 0.0001 mg/ml to 1000 mg/ml, and wherein said formulation has a pH from 2.0 to 10.0. The formulation may further comprise a buffer system, preservative(s), tonicity agent(s), chelating agent(s), stabilizers and surfactants. In one aspect of the invention the pharmaceutical formulation is an aqueous formulation, i.e. formulation comprising water. Such formulation is typically a solution or a suspension. In a further aspect of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another aspect the pharmaceutical formulation is a freeze-dried formulation, whereto the physician or the patient adds solvents and/or diluents prior to use.

In another aspect the pharmaceutical formulation is a dried formulation (e.g. freeze-dried or spray-dried) ready for use without any prior dissolution.

In a further aspect the invention relates to a pharmaceutical formulation comprising an aqueous solution of the FVIIa-derivative, and a buffer, wherein said FVIIa-derivative is present in a concentration from 0.01 mg/ml or above, and wherein said formulation has a pH from about 2.0 to about 10.0.

In a another aspect of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further aspect of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative aspect of the invention.

In a further aspect of the invention the formulation further comprises a pharmaceutically acceptable preservative. In a further aspect of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further aspect of the invention the preservative is present in a concentration from 0.1 mg/ml to 20 mg/ml. In a further aspect of the invention the preservative is present in a concentration from 0.1 mg/ml to 5 mg/ml. In a further aspect of the invention the preservative is present in a concentration from 5 mg/ml to 10 mg/ml. In a further aspect of the invention the preservative is present in a concentration from 10 mg/ml to 20 mg/ml. Each one of these specific preservatives constitutes an alternative aspect of the invention. The use of a preservative in pharmaceutical compositions is well-known to the skilled person. For convenience reference is made to Remington: The Science and Practice of Pharmacy, 19^{th} edition, 1995.

In a further aspect of the invention the formulation further comprises an isotonic agent. In a further aspect of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glycans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one aspect the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one aspect the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one aspect, the sugar or sugar alcohol concentration is between about 1 mg/ml and about 150 mg/ml. In a further aspect of the invention the isotonic agent is present in a concentration from 1 mg/ml to 50 mg/ml. In a further aspect of the invention the isotonic agent is present in a concentration from 1 mg/ml to 7 mg/ml. In a further aspect of the invention the isotonic agent is present in a concentration from 8 mg/ml to 24 mg/ml. In a further aspect of the invention the isotonic agent is present in a concentration from 25 mg/ml to 50 mg/ml. Each one of these specific isotonic agents constitutes an alternative aspect of the invention. The use of an isotonic agent in pharmaceutical compositions is well-known to the skilled person. For convenience reference is made to Remington: The Science and Practice of Pharmacy, 19^{th} edition, 1995.

In a further aspect of the invention the formulation further comprises a chelating agent. In a further aspect of the invention the chelating agent is selected from salts of ethylenediamine-tetraacetic acid (EDTA), citric acid, and aspartic acid, and mixtures thereof. In a further aspect of the invention the chelating agent is present in a concentration from 0.1 mg/ml to 5 mg/ml. In a further aspect of the invention the chelating agent is present in a concentration from 0.1 mg/ml to 2 mg/ml. In a further aspect of the invention the chelating agent is present in a concentration from 2 mg/ml to 5 mg/ml. Each one of these specific chelating agents constitutes an alternative aspect of the invention. The use of a chelating agent in pharmaceutical compositions is well-known to the skilled person. For convenience reference is made to Remington: The Science and Practice of Pharmacy, 19^{th} edition, 1995.

In a further aspect of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilised liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilisation; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one aspect, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one aspect the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further aspect of the invention the amino acids or amino acid analogues are used in a concentration, which is sufficient to prevent or delay aggregation of the peptide.

In a further aspect of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidised species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further aspect of the invention the formulation further comprises a stabilizer selected from the group of high molecular weight polymers or low molecular compounds. In a further aspect of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative aspect of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further aspect of the invention the formulation further comprises a surfactant. In a further aspect of the invention the surfactant is selected from a detergent, ethoxylated castor oil, polyglycolysed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative aspect of the invention.

The use of a surfactant in pharmaceutical compositions is well-known to the skilled person. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, peptides (e.g., human serum albumin, gelatine) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a FVIIa-derivative according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallisation, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the FVIIa-derivative, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallisation, condensation, co-crystallisation, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilised formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterised by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilised formulation” refers to a formulation with increased physical stability, increased chemical stability or increased physical and chemical stability. In general, a formulation must be stable during use and storage (in compliance with recommended use and storage conditions) until the expiration date is reached.

In one aspect of the invention the pharmaceutical formulation comprising the compound is stable for more than 6 weeks of usage and for more than 3 years of storage.

In another aspect of the invention the pharmaceutical formulation comprising the compound is stable for more than 4 weeks of usage and for more than 3 years of storage.

In a further aspect of the invention the pharmaceutical formulation comprising the compound is stable for more than 4 weeks of usage and for more than two years of storage.

In an even further aspect of the invention the pharmaceutical formulation comprising the compound is stable for more than 2 weeks of usage and for more than two years of storage. Specific examples of suitable protected monomers and building blocks included in the invention:

General formula Ia - Linear monomers (A—L1—X—L2—B):

| | |
|---|---|
| 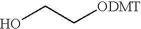 | 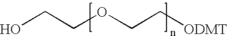 |

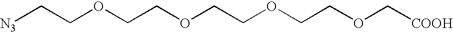

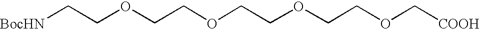

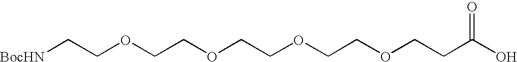

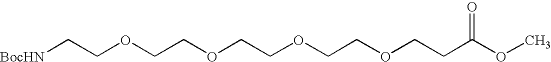

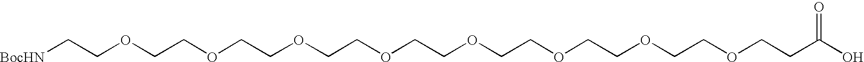

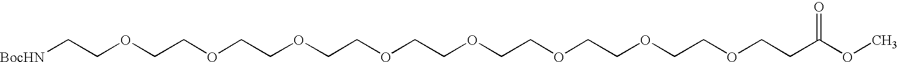

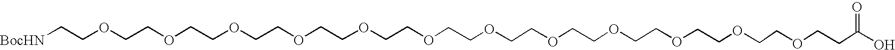

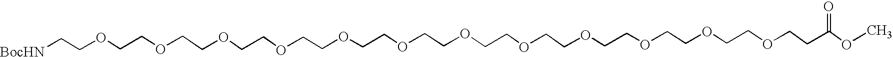

General formula Ib - Bifurcated monomers (A—L1—X—(L2—B)₂):

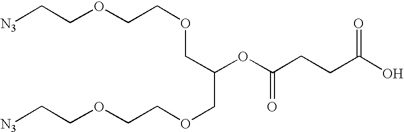

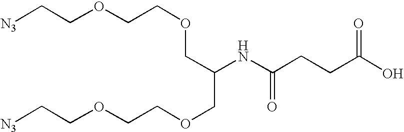

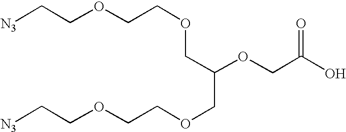

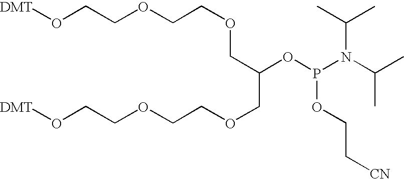

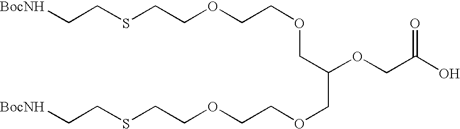

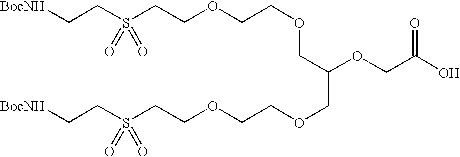

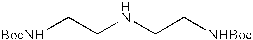

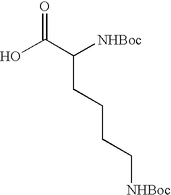

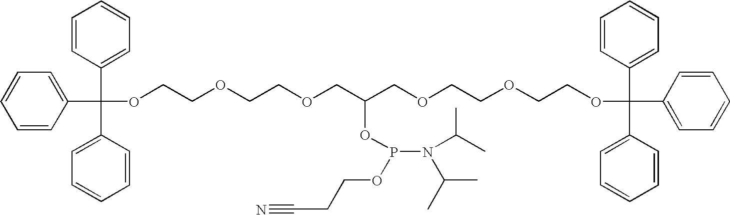

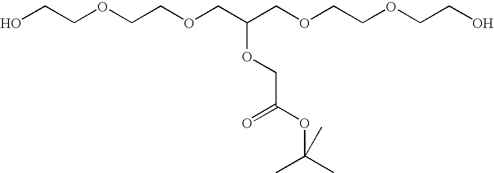

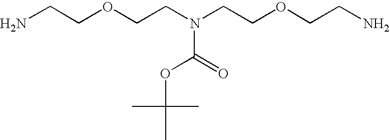

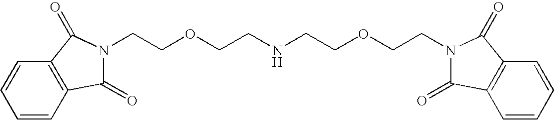

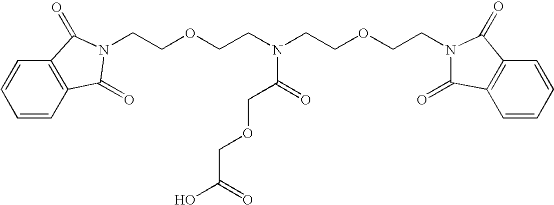

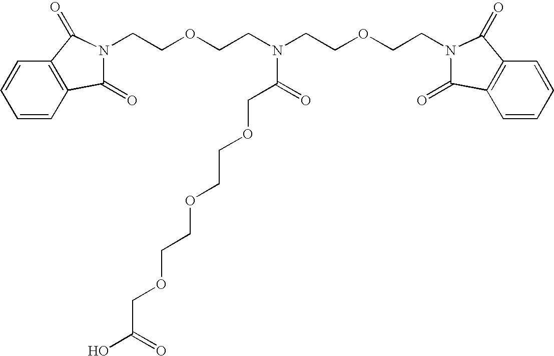

General formula Ic - Trifurcated monomers (A—L1—X—(L2—B)₃):

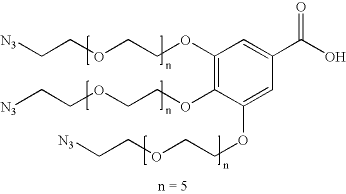

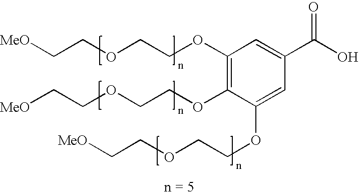

FIG. 1—Convergent Synthesis in Solution—Capped—First Generation 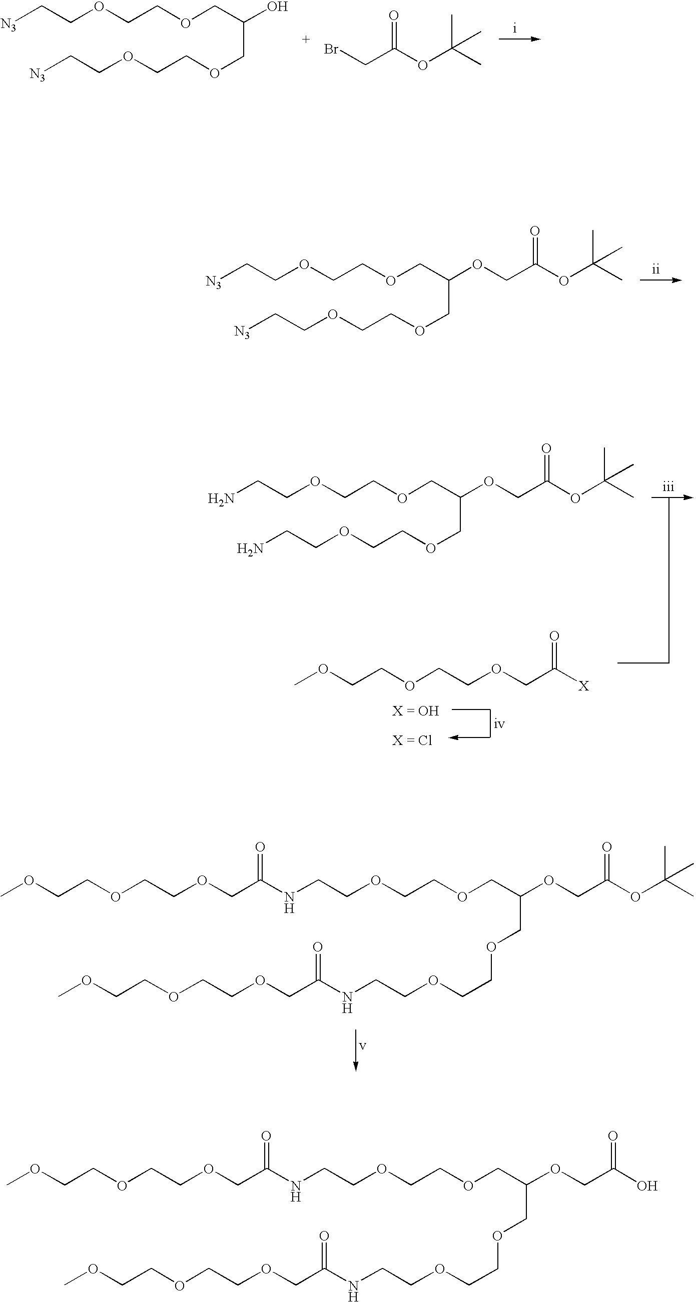
FIG. 2: Second Generation with Protected Focal Point 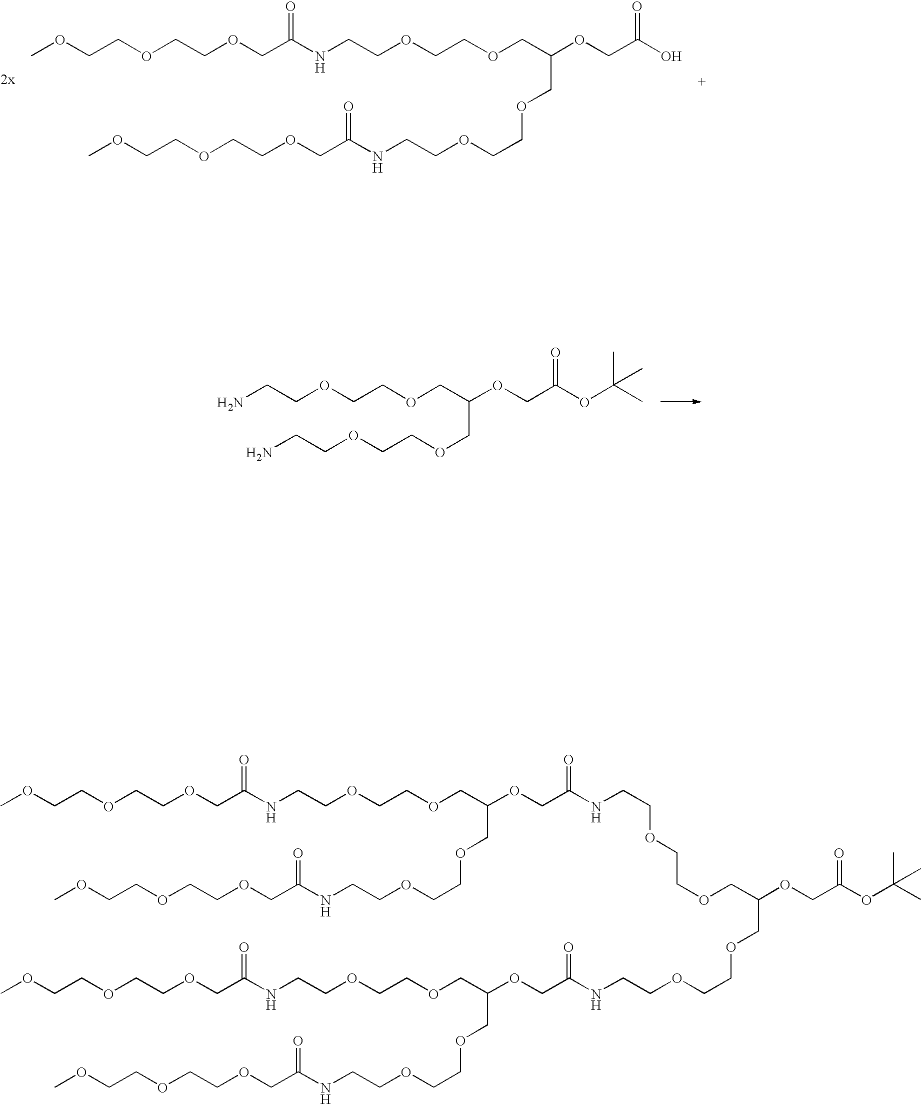
FIG. 3: Solid Phase Synthesis of a Second Generation Branched Polymer 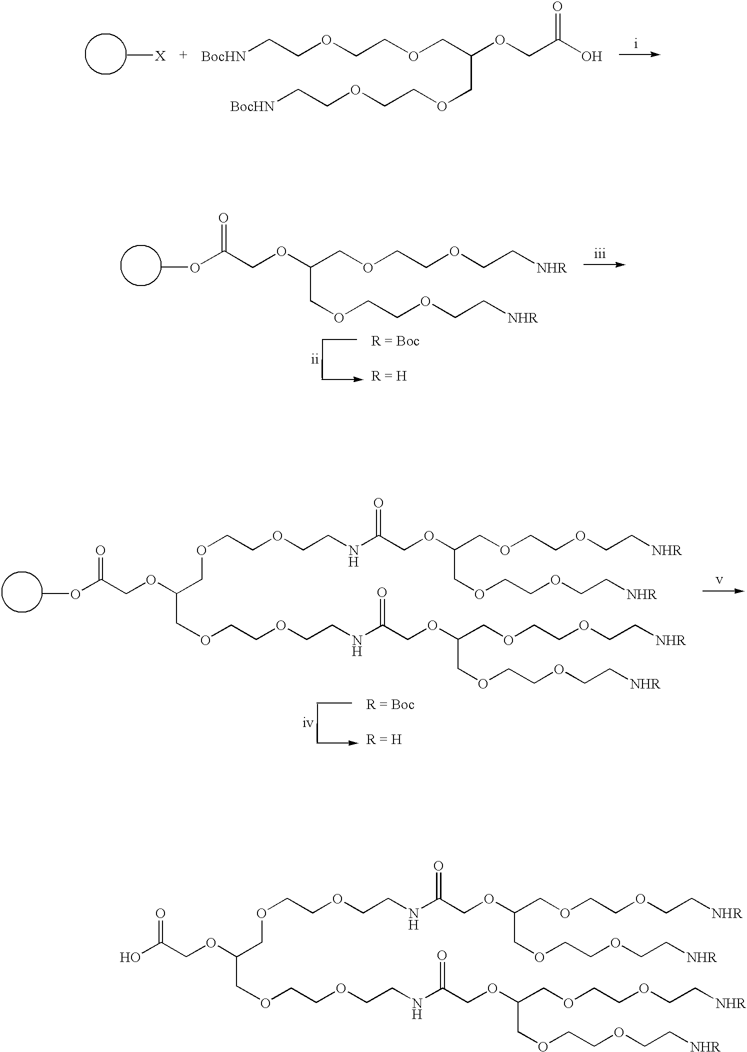
FIG. 4: Divergent Synthesis of a Second Generation Material in Solution 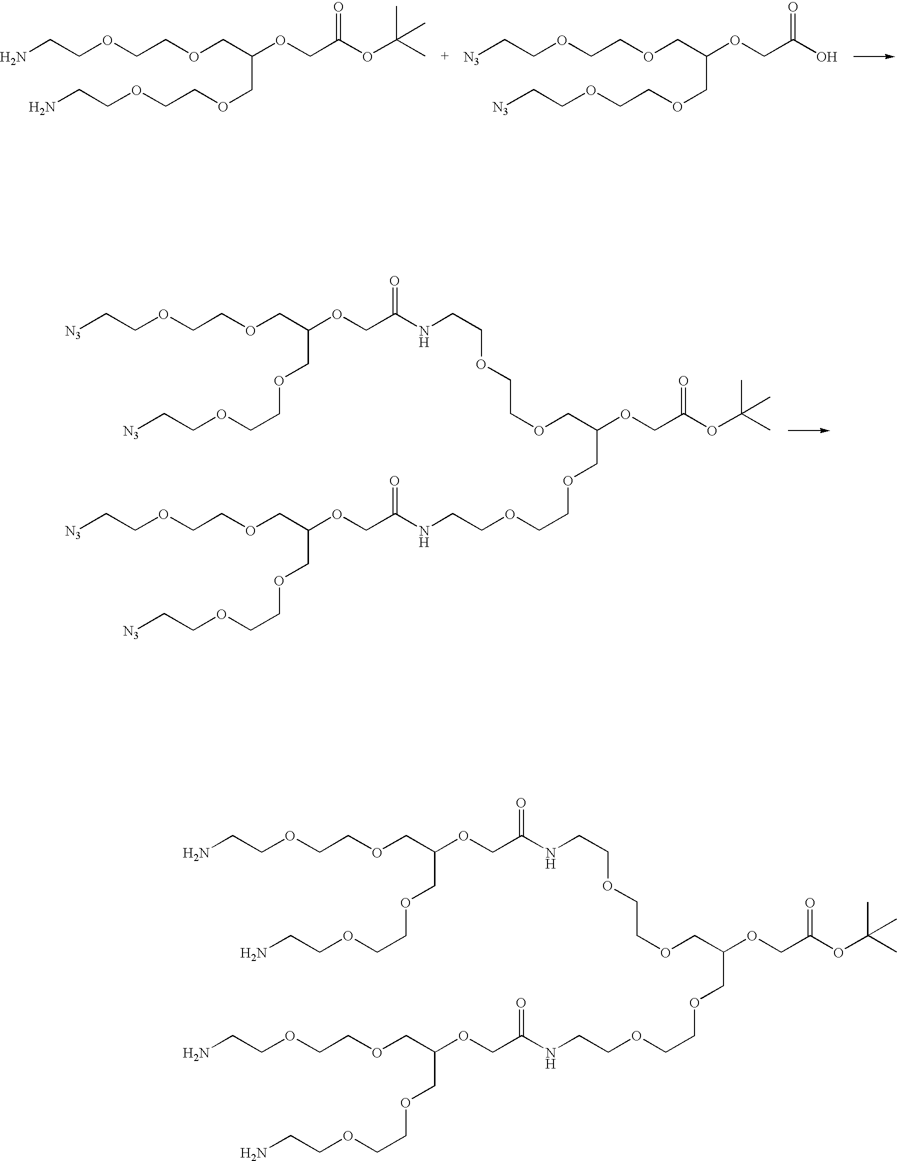
FIG. 5: Illustration of End Capping of a Second Generation Polymer Using a Me(PEG)2CH2COOH Acid 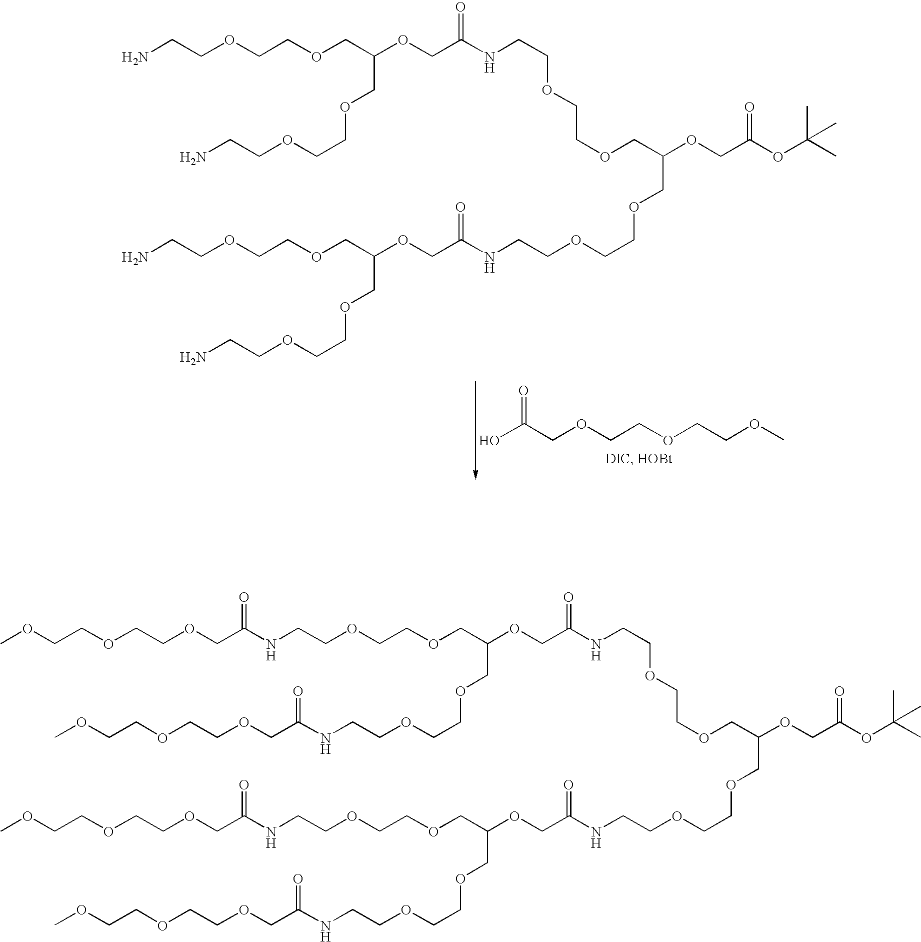
FIG. 6: Illustration of End Capping of a Second Generation Polymer Using Succinic Acid Mono Tert Butyl Ester to Create a Poly Anionic Glyco Mimic Polymer. 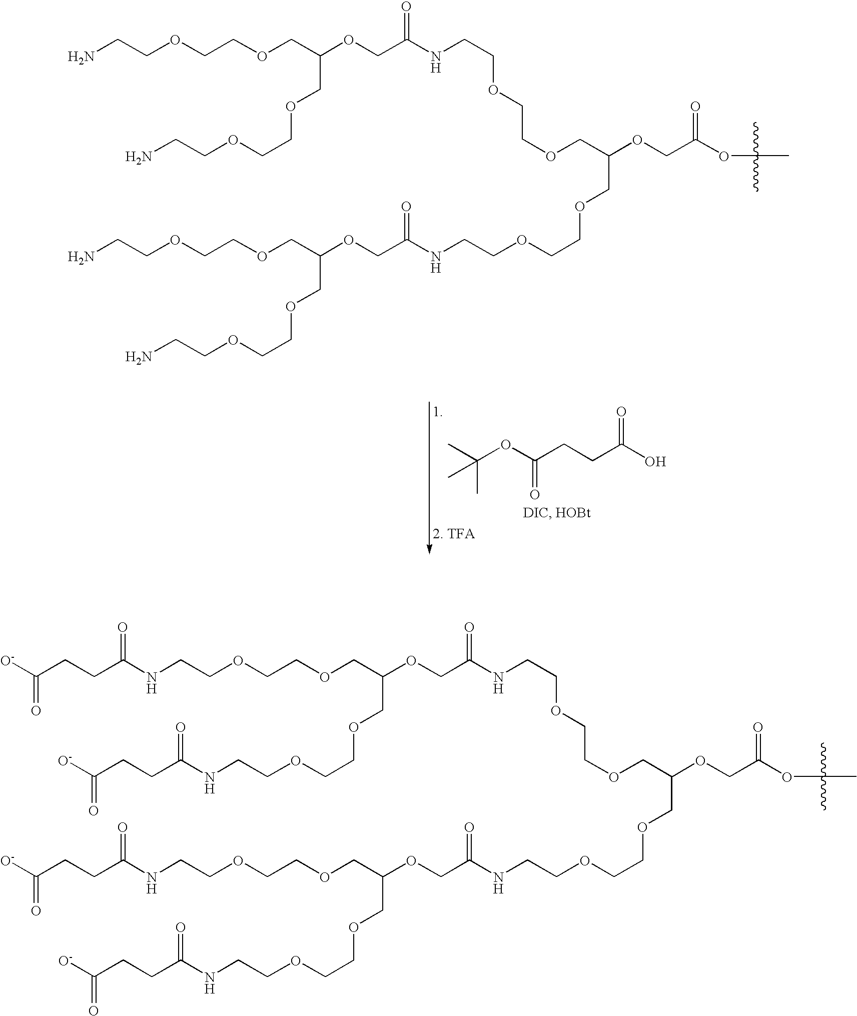
FIG. 7: Formation of Suitable Reactive Handle for Peptide Conjugation. Illustrated for a Second Generation Polymer Material.

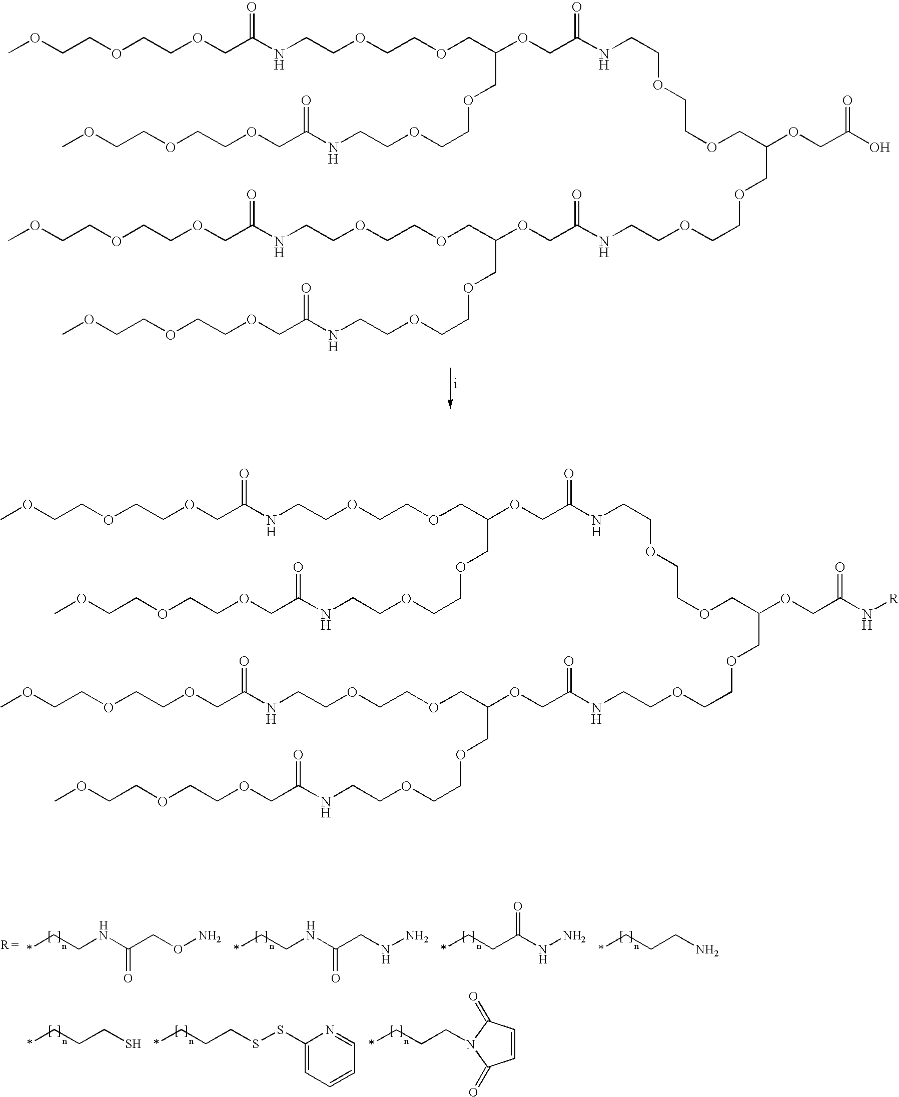

FIG. 8: General Scheme for Convergent Oligomerization of a Monomer Described According to General Formula I

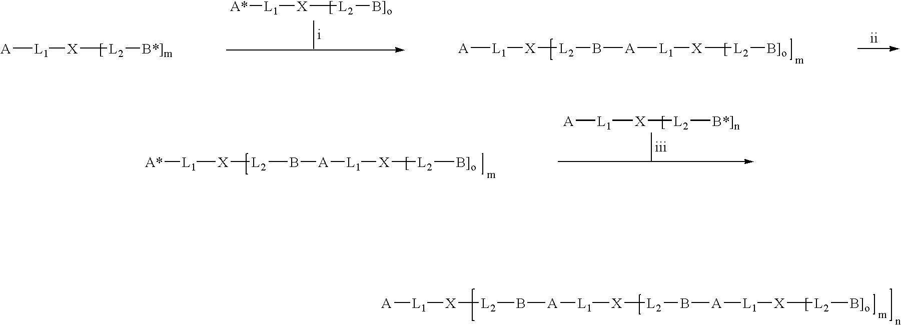

Convergent synthesis (illustrated for the synthesis of a 3-generation dendrimer): Step i) coupling of monomer A*-L1-X-(L2-B)ₒ to B* of monomer A-L1-X-(L2-B*)ₘ, where A* is activated or deprotected A, and B* is activated or deprotected B. Step ii: activation or deprotection of focal A to A*. Step iii: coupling to B* of monomer A-L1-X-(L2-B*)ₙ.

FIG. 9: General Scheme for Divergent Oligomerization of a Monomer Described According to General Formula I

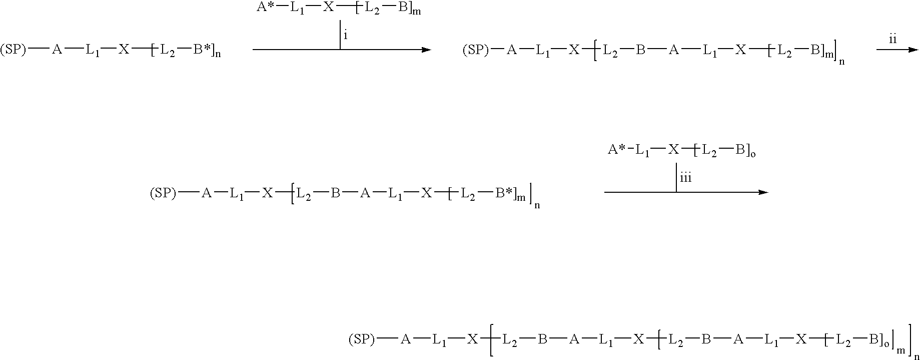

Divergent synthesis (illustrated for the synthesis of a 3-generation dendrimer): Step i) coupling of monomer A*-L1-X-(L2-B)ₘ to B* of monomer A-L 1-X-(L2-B*)ₙ, where A* is activated or deprotected A, and B* is activated or deprotected B. Step ii: activation or deprotection of terminal B to B*. Step iii: coupling of monomer A*-L1-X-(L2-B)₀. In the divergent method, the first monomer may optionally be attatched to a solid support (SP).

### EXAMPLES

The following examples and general procedures refer to intermediate compounds and final products identified in the structural specification and in the synthesis schemes. The preparation of the compounds of the present invention is described in detail using the following examples, but the chemical reactions described are disclosed in terms of their general applicability to the preparation of selected branched polymers of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alter-natively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents. All reagents were of standard grade as supplied from Aldrich, Sigma, ect. Proton, carbon and phosphor nuclear magnetic resonance (¹H-, ¹³C- and ³¹P-NMR) were recorded on a Bruker NMR apparatus, with chemical shift (δ) reported down field from tetramethylsilane or phosphoric acid. LC-MS mass spectra were obtained using apparatus and setup conditions as follows:
Hewlett Packard series 1100 G1312A Bin Pump
Hewlett Packard series 1100 Column compartment
Hewlett Packard series 1100 G13 15A DAD diode array detector
Hewlett Packard series 1100 MSD

The instrument was controlled by HP Chemstation software.

The HPLC pump was connected to two eluent reservoirs containing:
A: 0.01% TFA in water
B: 0.01% TFA in acetonitrile

The analysis was performed at 40° C. by injecting an appropriate volume of the sample (preferably 1 μL) onto the column, which was eluted with a gradient of acetonitrile.

The HPLC conditions, detector settings and mass spectrometer settings used are given in the following table.

| | |
|---|---|
| Column | Waters Xterra MS C-18, 5 um, 50 × 3 mm id |
| Gradient | 10%-100% acetonitrile lineary during 7.5 min at 1.0 ml/min |
| Detection | UV: 210 nm (analog output from DAD) |
| MS | Ionisation mode: API-ES |
| | Scan 100-1000 amu step 0.1 amu |

Some of the NMR data shown in the following examples are only selected data.
In the examples the following terms are intended to have the following, general meanings:
Abbreviations
Boc: tert-butoxycarbonyl
CDI: carbonyldiimidazole
DCM: dichloromethane, methylenechloride
DIC: diisopropylcarbodiimide
DIPEA: N,N-diisopropylethylamine
DhbtOH: 3-hydroxy-1,2,3-benzotriazin-4(3H)-one
DMAP: 4-dimethylaminopyridine
DMF: N,N-dimethylformamide
DMSO: dimethyl sulphoxide
DTT: Dithiothreitol
EtOH: ethanol
Fmoc: 9-fluorenylmethyloxycarbonyl
HOBt: 1-hydroxybenzotriazole
MeOH: methanol
NMP: N-methyl-2-pyrrolidinone
NEt₃: triethylamine
THF: tetrahydrofuran
TFA: trifluoroacetic acid
TSTU: 2-succinimido-1,1,3,3-tetramethyluronium tetrafluoroborate

The following non limiting examples illustrates the synthesis of monomers and polymerisation technique using solid phase synthesis or solution phase synthesis.

Synthesis of Monomer Building Blocks and Linkers

### Example 1

### 2-[2-(2-Chloroethoxy)ethoxymethyl]oxirane

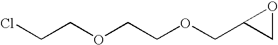

2-(2-Chloroethoxy)ethanol (100.00 g; 0.802 mol) was dissolved in dichloromethane (100 ml) and a catalytical amount of boron trifluride etherate (2.28 g; 16 mmol). The clear solution was cooled to 0° C., and epibromhydrin (104.46 g; 0.762 mol) was added dropwise maintaining the temperature at 0° C. The clear solution was stirred for an additional 3 h at 0° C., then solvent was removed by rotary evaporation. The residual oil was evapoprated once from acetonitrile, to give crude 1-bromo-3-[2-(2-chloroethoxy)ethoxy]propan-2-ol, which was re-dissolved in THF (500 ml). Powdered potassium tert-butoxide (85.0 g; 0.765 mmol) was then added, and the mixture was heated to reflux for 30 min. Insoluble salts were removed by filtration, and the filtrate was concentrated, in vacuo, to give a clear yellow oil. The oil was further purified by vacuum destillation, to give 56.13 g (41%) of pure title material.

bp=65-75° C. (0.65 mbar). ¹H-NMR (CDCl₃): δ 2.61 ppm (m, 1H); 2.70 (m, 1H); 3.17 (m, 1H); 3.43 (dd, 1H); 3.60-3.85 (m, 9H). ¹³C-NMR (CDCl₃): δ 42.73 ppm; 44.18; 50.80; 70.64 & 70.69 (may collaps); 71.37; 72.65.

### Example 2

### 1,3-Bis[2-(2-chloroethoxy)ethoxy]propan-2-ol

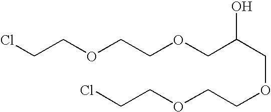

2-[2-(2-Chloroethoxy)ethoxymethyl]oxirane (2.20 g; 12.2 mmol) was dissolved in DCM (20 ml), and 2-(2-chloroethoxy)ethanol (1.52 g; 12.2 mol) was added. The mixture was cooled to 0° C. and a catalytical amount of boron trifluride etherate (0.2 ml; 1.5 mmol) was added. The mixture was stirred at 0° C. for 2 h, then solvent was removed by rotary evaporation. Residual of boron trifluride etherate was removed by co-evaporating twice from acetonitril. The oil thus obtained was purified by kuglerohr destilation. The title material was obtained as a clear viscous oil in 2.10 g (45%) yield. bp.=270° C., 0.25 mbar. ¹H-NMR (CDCl₃): δ 3.31 (bs, 1H); 3.55 ppm (ddd, 4H); 3.65-3.72 (m, 12H); 3.75 (t, 4H); 3.90 (m, 1H). ¹³C-NMR (CDCl₃): δ 43.12 ppm; 69.92; 70.95; 71.11; 71.69; 72.69.

### Example 3

### 1,3-Bis[2-(2-azidoethoxy)ethoxy]propan-2-ol

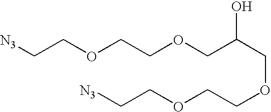

1,3-Bis[2-(2-chloroethoxy)ethoxy]propan-2-ol (250 mg; 0.81 mmol) was dissolved in DMF (2.5 ml), and sodium azide (200 mg; 3.10 mmol) and sodium iodide (100 mg; 0.66 mmol) were added. The suspension was heated to 100° C. (internal temperature) over night. The mixture was then cooled and filtered. The filtrate was taken to dryness, and the semi crystalline oil resuspended in DCM (5 ml). The non-soluble salts were removed by filtration; the filtrate was evaporated to dryness to give pure title mateial as a colorless oil. Yield: 210 mg (84%). ¹H-NMR (CDCl₃): δ 3.48 ppm (t, 4H); 3.60-3.75 (m, 16H); 4.08 (m, 1H). ¹³C-NMR (CDCl₃): δ 51.05 ppm; 69.10; 70.24; 70.53; 70.78; 71.37. LC-MS (any-one): m/e=319 (M+1)⁺; 341 (M+Na)⁺; 291 (M−N₂)⁺. Rₜ=2.78 min.

### Example 4

### 1,3-Bis[2-(2-azidoethoxy)ethoxy]propan-2-yl-p-nitrophenylcarbonate

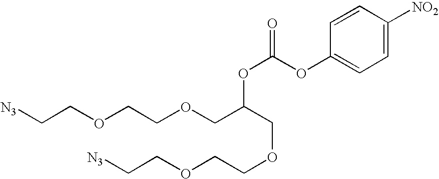

1,3-Bis[2-(2-azidoethoxy)ethoxy]propan-2-ol (2.00 g; 6.6 mmol) was dissolved in THF (50 ml) and diisopropylethylamine (10 ml) was added. The clear yellow solution was then added 4-dimethylaminopyridine (1.60 g; 13.1 mmol) and p-nitrophenylchloroformiate (2.64 g; 13.1 mmol) and stirred at ambient temperature. A precipitate rapidly formed. The suspension was stirred for 5 h at room temperature, then filtered and concentrated in vacuo. The residue was further purified by chromatography using ethylacetate-heptane-triethylamine (40/60/2) as eluent. The product was obtained as a clear yellow oil in 500 mg (16%) yield. ¹H-NMR (CDCl₃): δ 3.38 ppm (t, 4H); 3.60-3.72 (m, 12H); 3.76 (m, 4H); 5.12 (q, 1H); 7.41 (d, 2H); 8.28 (d, 2H). LC-MS (anyone): m/e=506 (M+Na)⁺; 456 (M-N₂)⁺. Rₜ=4.41 min.

### Example 5

### 1,3-Bis[2-(2-azidoethoxy)ethoxy]propan-2-yl chloroformiate

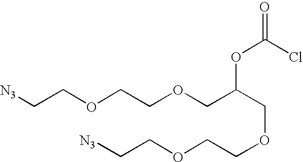

Trichloroacetylchloride (1.42 g, 7.85 mmol) was dissolved in THF (10 ml), and the solution was cooled to 0° C. A solution of 1,3-Bis[2-(2-azidoethoxy)ethoxy]propan-2-ol (1.00 g; 3.3 mmol) and triethylamine (0.32 g, 3.3 mmol) in THF (5 ml) was slowly added drop wise over 10 min. Cooling was removed, and the resulting suspension was stirred for 6 h at ambient temperature. The mixture was filtered, and the filtrate was evaporated to give a light brown oil. The oil was treated twice with acetonitril following evaporation, and the product was used without further purification.

¹H-NMR (CDCl₃): δ 3.40 (t, 4H); 3.55-3.71 (m, 12H); 3.75 (d, 4H); 5.28 (m, 1H).

### Example 6

### 2-(1,3-Bis[2-(2-azidoethoxy)ethoxy]propan-2-yloxy)acetic acid

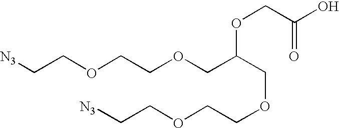

Sodium hydride (7.50 g; 80% oil suspension) was washed trice with heptanes, and then resuspended in dry THF (100 ml). A solution of 1,3-bis[2-(2-azidoethoxy)ethoxy]propan-2-ol (10.00 g; 33.0 mmol) in dry THF (100 ml) was then slowly added over a period of 30 min at room temperature. Then a solution of bromo acetic acid (6.50 mg; 47 mmol) in THF (100 ml) was added drop wise over 20 min.->slight heat evolution. A cream coloured suspension was formed. The mixture was stirred at ambient temperature over night. Excess sodium hydride was carefully destroyed by addition of water (20 ml) while cooling the mixture. The suspension was taken to dryness by rotary evaporation, and the residue partitioned between DCM and water. The water phase was extracted twice with DCM then acidified by addition of acetic acid (25 ml). The water phase was then extracted twice with DCM, and the combined organic phases were dried over sodium sulphate, and evaporated to dryness. The residual oil at this point contained the title material as well as bromo acetic acid. The later was removed by re-dissolving the oil in DCM (50 ml) containing piperidine (5 ml); stir for 30 min., and then wash of the organic solution trice with 1N aquoeus HCl (3×). Pure title material was then obtained after drying (Na2SO4) and evaporation of the solvent. Yield: 7.54 g (63%).

¹H-NMR (CDCl₃): δ 3.48 ppm (t, 4H); 3.55-3.80 (m, 16H); 4.28 (s, 2H); 4.30 (m, 1H); 8.50 (bs, 1H). ¹³C-NMR (CDCl₃): δ 51.04 ppm; 69.24; 70.50; 70.72; 71.39; 71.57; 80.76; 172.68. LC-MS (any-one): m/e=399 (M+Na)⁺; 349 (M−N₂)⁺. Rₜ=2.34 min.

### Example 7

### Imidazole-1-carboxylic acid 1,3-bis(2-(2-azidoethoxy)ethoxy)propan-2-yl ester

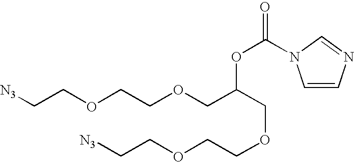

1,3-Bis[2-(2-azidoethoxy)ethoxy]propan-2-ol (1.00 g; 3.3 mmol) was dissolved in DCM (5 ml) and carbonyl diimidazole (1.18 g, 6.3 mmol) was added. The mixture was stirred for 2 h at room temperature. Solvent was removed and the residue was dissolved in methanol (20 ml) and stirred for 20 min. Solvent was removed and the clear oil, thus obtained was further purified by column chromatography on silica using 2% MeOH in DCM as eluent. Yield: 372.4 mg (35%). ¹H-NMR (CDCl₃): δ 3.33 (t, 4H); 3.60-3.75 (m, 12H); 3.80 (d, 4H); 5.35 (m, 1H); 7.06 (s, 1H); 7.43 (s, 1H); 8.16 (s, 1H).

LC-MS (any-one): m/e=413 (M+1)⁺; Rₜ=2.35 min.

### Example 8

### t-Butyl 2-(1,3-bis[2-(2-azidoethoxy)ethoxy]propan-2-yloxy)acetate

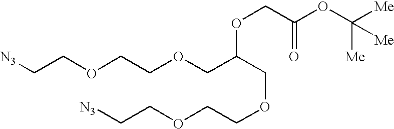

2-(1,3-Bis[2-(2-azidoethoxy)ethoxy]propan-2-yloxy)acetic acid (5.0 g; 13.28 mmol) was dissolved in toluene (20 ml), and the reaction mixture was heated to reflux under an inert atmosphere. N,N-dimethylformamid-di-tert-butylacetal (13 ml; 54.21 mmol) was then added dropwise over 30 min. Reflux was continued for 24 h. The dark brown solution was then filtered through Celite. Solvent was removed under vacuum, and the oily residue was purified by flash chromatography on silica, using 3% methanol dichloromethane as eluent. Pure fractions were pooled and evaporated to dryness. The title material was obtained as a yellow clear oil. Yield: 5.07 g (88%). ¹H-NMR (CDCl₃): δ 1.42 ppm (s, 9H); 3.35 (t, 4H); 3.54-3.69 (m, 16H); 3.75-3.85 (m, 1H); 4.16 (s, 2H). ¹³C-NMR (CDCl₃, selected peaks): δ 30.35 ppm.; 52.93; 70.65; 72.25; 73.12; 73.90; 80.44; 83.55; 172.28. R_{f}=0.33 in ethyl acetate-heptane (1:1).

### Example 9

### t-Butyl 2-(1,3-bis[2-(2-aminoethoxy)ethoxy]propan-2-yloxy)acetate

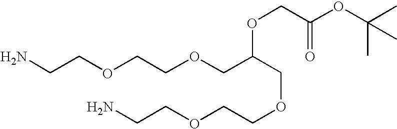

t-Butyl 2-(1,3-bis[2-(2-azidoethoxy)ethoxy]propan-2-yloxy)acetate (5.97 g, 11.7 mmol) was dissolved in ethanol-water (25 ml; 2:1), and acetic acid (5 ml) was added, followed by a aqueous suspension of Raney-Nickel (5 ml). The mixture was then hydrogenated at 3 atm., for 16 h using a Parr apparatus. The catalyst was then removed by filtration, and the reaction mixture was taken to dryness by rotary evaporation. The oily residue was dissolved in water and freeze dried to give a quantitative yield of title material. ¹H-NMR (CDCl₃): δ 1.45 ppm (s, 9H); 3.15 (bs, 4H); 3.48-3.89 (broad m, 17H); 4.15 (s, 2H). ¹³C-NMR (CDCl₃, selected peaks): δ 28.44 ppm.; 39.81; 68.17; 70.58; 70.79; 70.99; 78.81; 82.31; 170.59.

### Example 10

### 2-(1,3-Bis[2-(2-aminoethoxy)ethoxy]propan-2-yloxy)acetic acid

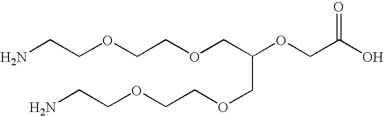

2-(1,3-Bis[2-(2-azidoethoxy)ethoxy]propan-2-yloxy)acetic acid (1.00 g; 2.65 mmol) was dissolved in 1N aqueous hydrochloric acid (10 ml) and a 50% aqueous suspension of 5% palladium on carbon (1 ml) was added. The mixture was hydrogenated at 3.5 atm using a Parr apparatus. After one hour the reaction was stopped, and the catalyst removed by filtration. The solvent was removed by rotary evaporation, and the residue was evaporated twice from acetonitril. Yield: 930 mg (88%). ¹H-NMR (D₂O): 63.11 ppm (t, 4H); 3.53-3.68 (m, 16H); 3.80 (m, 1H); 4.25 (s, 2H). ¹³C-NMR (D₂O): δ 38.18 ppm.; 65.43; 66.09; 68.55: 69.13; 69.23; 77.18; 173.42.

### Example 11

### 2-(1,3-Bis[2-(2-{9-fluorenylmethyloxycarbonylamino}ethoxy)ethoxy]propan-2-yloxy)acetic acid

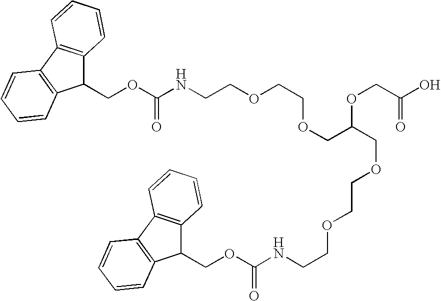

2-(1,3-bis[2-(2-aminoethoxy)ethoxy]propan-2-yloxy)acetic acid (9.35 g; 28.8 mmol) was added DIPEA (10 ml; 57 mmol). The reaction mixture was cooled on an ice bath, and chlorotrimethylsilane (15 ml; 118 mmol) dissolved in DCM (50 ml) was added dropwise, followed by DIPEA (11 ml; 62.7 mmol). To the almost clear solution was added dropwise a solution of Fmoc-Cl (15.0 g; 57 mmol) in DCM (50 ml). The reaction mixture was stirred overnight, then diluted with DCM (500 ml) and added to 0.01 N aqueous solution (500 ml). The organic layer was separated; washed with water (3×200 ml) and dried over anhydrous sodium sulfate. Solvent was removed by rotary evaporation. The crude product was purified by flash chromatography on silica using ethylacetate-heptane (1:1) as eluent. Pure fractions were collected and taken to dryness to give 9.20 g (42%) of title material.

¹H-NMR (D₂O): δ 3.34 ppm (t, 4H); 3.45-3.65 (m, 16H); 3.69 (bs, 1H); 4.20 (t, 2H); 4.26 (s, 2H); 4.38 (d, 4H); 5.60 (t, 2H); 7.30 (t, 4H); 3.35 (t, 4H); 7.58 (d, 4H); 7.72 (d, 4H). ¹³C-NMR (D₂O; selected peaks): δ 21.20 ppm.; 30.75; 34.64; 67.66; 68.90; 70.38; 70.51; 80.02; 120.37; 125.54; 127.48; 128.09; 128.67; 136.27; 141.69; 173.63; 176.80.

### Example 12

### 2-[2-(2-azidoethoxy)ethoxy]ethanol

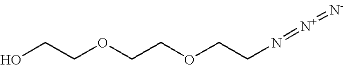

A slurry of 2-(2-(−2-chloroethoxy)ethoxy)ethanol (25.0 g, 148 mmol) and sodiumazide (14.5 g, 222 mmol) in dimethylformamide (250 ml) was standing at 100° C. night over. The reaction mixture was cooled on an ice bath, filtered and the organic solvent was evaporated in vacuo. The residue was dissolved in dichloromethane (200 ml), washed with water (75 ml), the water-phase was extracted with additional dichloromethane (75 ml) and the combined organic phases were dried with magnesium sulphate (MgSO₄), filtered and evaporated in vacuo giving an oil which was used without further purification. Yield: 30.0 g (100%). ¹³C-NMR (CDCl₃): δ 72.53; 70.66-70.05; 61.74; 50.65

### Example 13

### (2-[2-(2-Azidoethoxy)ethoxy]ethoxy)acetic acid

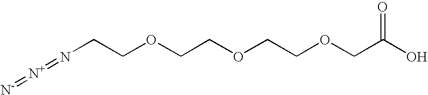

The above 2-[2-(2-azidoethoxy)ethoxy]ethanol (26 g, 148 mmol) was dissolved in tetrahydrofurane (100 ml) and under an nitrogen atmosphere slowly added to an ice cooled slurry of sodium hydride (24 g, 593 mmol, 60% in oil)) (which in advance had been washed with heptane (2×100 ml)) in tetrahydrofurane (250 ml). The reaction mixture was standing for 40 min. then cooled on a ice bath followed by slowly addition of bromoacetic acid (31 g, 223 mmol) dissolved in tetrahydrofurane (150 ml) and then standing about 3 hours at RT. The organic solvent was evaporated in vacuo. The residue was suspended in dichloromethane (400 ml). Water (100 ml) was slowly added, whereafter the mixture was standing for 30 min. under mechanical stirring. The water phase was separated, acidified with hydrochloride (4N) and extracted with dichloromethane (2×75 m[). All the combined organic phases were evaporated in vacuo giving a yellow oil. To the oil was slowly added a solution of piperidine (37 ml, 371 mmol) in dichloromethane (250 ml), the mixture was standing under mechanical stirring for 1 hour. The clear solution was diluted with dichloromethane (100 ml) and washed with hydrochloride (4N, 2×100 ml). The water phase was extracted with additional dichloromethane (2×75 ml) and the combined organic phases were evaporated in vacuo, giving an yellow oil which was used without further purification. Yield: 27.0 g (66%). ¹³C-NMR (CDCl₃): δ 173.30; 71.36; 70.66-70.05; 68.65; 50.65

### Example 14

### (S)-2,6-Bis-(2-{2-[2-(2-azidoethoxy)ethoxy]ethoxy}acetylamino)hexanoic acid methyl ester

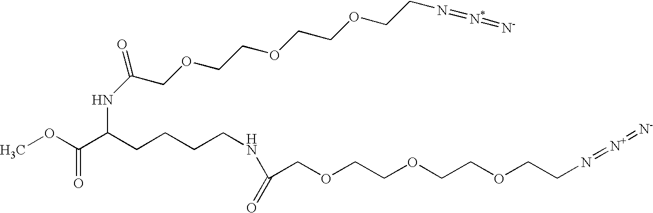

The above (2-[2-(2-azidoethoxy)ethoxy]ethoxy)acetic acid (13 g, 46.9 mol) was dissolved in dichloromethane (100 ml). N-Hydroxysuccinimide (6.5 g, 56.3 mmol) and 1-ethyl-3-(3-dimethylaminopropylcarbodiimide hydrochloride (10.8 g, 56.3 mmol) was added and the reaction mixture was standing for 1 hour. Diisopropylethylamine (39 ml, 234 mmol) and L-lysine methyl ester dihydrochloride (6.0 g, 25.8 mmol) were added and the reaction mixture was standing for 16 hours. The reaction mixture was diluted with dichloromethane (300 ml), extracted with water (100 ml), hydrochloride (2N, 2×100 ml), water (100 ml), 50% saturated sodiumhydrogencarbonate (100 ml) and water (2×100 ml). The organic phase was dried with Magnesium sulphate, filtered and evaporated in vacuo, giving an oil, which was used without further purification. Yield: 11 g (73%). LCMS: m/z=591. ¹³C-NMR (CDCl₃): (selected) δ 172.48; 169.87; 169.84; 71.093-70.02; 53.51; 52.34; 51.35; 50.64; 38.48; 36.48; 31.99; 31.40; 29.13; 22.82

### Example 15

### (S)-2,6-Bis-(2-{2-[2-(2-t-butyloxycarbonylaminoethoxy)ethoxy]ethoxy}acetylamino)hexanoic acid methyl ester

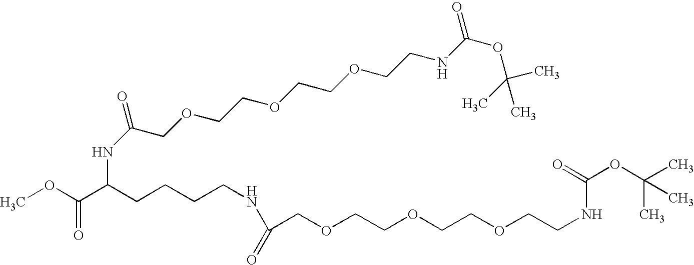

To a solution of the above (S)-2,6-bis-(2-{2-[2-(2-azidoethoxy)ethoxy]ethoxy}acetylamino)hexanoic acid methyl ester (1.0 g, 1.7 mmol) in ethylacetate (15 ml) was added di-tert-butyl dicarbonat (0.9 g, 4.24 mmol) and 10% Pd/C (0.35 g). Hydrogen was then constantly bubbled through the solution for 3 hours. The reaction mixture was filtered and the organic solvent was removed in vacuo. The residue was purified by flash chromatography using ethylacetate/methanol 9:1 as the eluent. Frations containing product were pooled and the organic solvent was removed in vacuo giving an oil. Yield: 0.60 g (50%). LC-MS: m/z=739 (M+1).

### Example 16

### (S)-2,6-Bis-(2-{2-[2-(2-aminoethoxy)ethoxy]ethoxy}acetylamino)hexanoic acid methyl ester

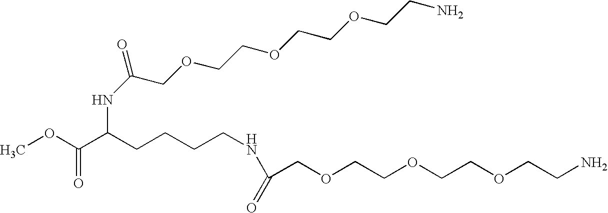

The above (S)-2,6-bis-(2-{2-[2-(2-t-butyloxycarbonylaminoethoxy)ethoxy]ethoxy}acetylamino)hexanoic acid methyl ester (0.6 g, 0.81 mmol) was dissolved in dichloromethane (5 ml). Trifluoroacetic acid (5 ml) was added and the reaction mixture was standing about 1 hour. The reaction mixture was evaporated, in vacuo, giving an oil, which was used without further purification. Yield: 0.437 g (100%). LC-MS m/z=539 (M+1)

### Example 17

### (S)-2,6-Bis-(2-{2-[2-(2-azidoethoxy)ethoxy]ethoxy}acetylamino)hexanoic acid

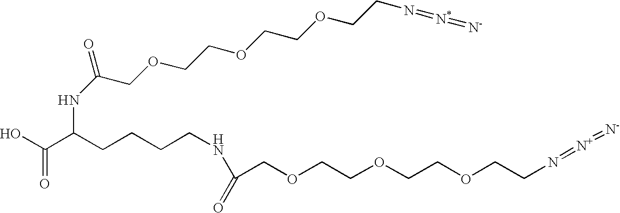

To a solution of (S)-2,6-bis-(2-{2-[2-(2-azidoethoxy)ethoxy]ethoxy}acetylamino)hexanoic acid methyl ester (2.0 g, 3.47 mmol) in methanol (10 ml) was added sodiumhydroxide (4N, 1.8 ml, 6.94 mmol) and the reaction mixture was standing for 2 hours. The organic solvent was evaporated in vacuo, and the residue was dissolved in water (45 ml) and acidified with hydrogenchloride (4N). The mixture was extracted with dichloromethane (150 ml) which was washed with saturated aqueous sodiumchloride (2×25 ml). The organic phase was dried over magnesium sulphate, filtered and evaporated, in vacuo, giving an oil. LC-MS m/z=577 (M+1).

### Example 18

### N-(tert-Butyloxycarbonylaminoxybutyl)phthalimide

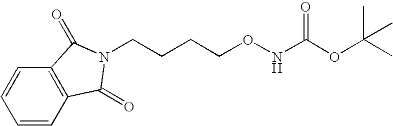

To a stirred mixture of N-(4-bromobutyl)phthalimide (18.9 g, 67.0 mmol), MeCN (14 ml), and N-Boc-hydroxylamine (12.7 g, 95.4 mmol) was added DBU (15.0 ml, 101 mmol) in portions. The resulting mixture was stirred at 50° C. for 24 h. Water (300 ml) and 12 M HCl (10 ml) were added, and the product was extracted three times with AcOEt. The combined extracts were washed with brine, dried (MgSO₄), and concentrated under reduced pressure. The resulting oil (28 g) was purified by chromatography (140 g SiO₂, gradient elution with heptane/AcOEt). 17.9 g (80%) of the title compound was obtained as an oil. ¹H NMR (DMSO-d₆) δ 1.36 (s, 9H), 1.50 (m, 2H), 1.67 (m, 2H), 3.58 (t, J=7 Hz, 2H), 3.68 (t, J=7 Hz, 2H), 7.85 (m, 4H), 9.90 (s, 1H).

### Example 19

### 4-(tert-Butyloxycarbonylaminoxy)butylamine

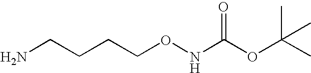

To a solution of N-(tert-butyloxycarbonylaminoxybutyl)phthalimide (8.35 g, 25.0 mmol) in EtOH (10 ml) was added hydrazine hydrate (20 ml), and the mixture was stirred at 80° C. for 38 h. The mixture was concentrated and the residue coevaporated with EtOH and PhMe. To the residue was added EtOH (50 ml), and the precipitated phthalhydrazide was filtered off and washed with EtOH (50 ml). Concentration of the combined filtrates yielded 5.08 g of an oil. This oil was mixed with a solution of K₂CO₃ (10 g) in water (20 ml), and the product was extracted with CH₂Cl₂. Drying (MgSO₄) and concentration yielded 2.28 g (45%) of the title compound as an oil, which was used without further purification. ¹H NMR (DMSO-d₆) δ 1.38 (m, 2H), 1.39 (s, 9H), 1.51 (m, 2H), 2.51 (t, J=7 Hz, 2H), 3.66 (t, J=7 Hz, 2H).

### Example 20

### 2-(2-Trityloxyethoxy)ethanol

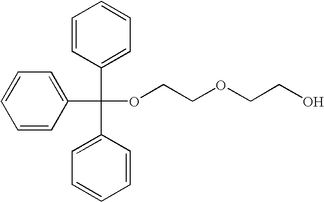

Tritylchloride (10 g, 35.8 mmol) was dissolved in dry pyridine, diethyleneglycol (3.43 mL, 35.8 mmol) was added and the mixture was stirred under nitrogen overnight. The solvent was removed in vacuo. The residue was dissolved in dichloromethane (100 mL) and washed with water. The organic phase was dried over Na₂SO₄ and solvent was removed in vacuo. The crude product was purified by recrystallisation from heptane/toluene (3:2) to yield the title compound.

¹H NMR (CDCl₃): δ 7.46 (m, 6H), 7.28, (m, 9H), 3.75 (t, 2H), 3.68 (t, 2H), 3.62 (t, 2H), 3.28 (t, 2H). LC-MS: m/z=371 (M+Na); Rₜ=2.13 min.

### Example 21

### 2-[2-(2-Trityloxyethoxy)ethoxymethyl]oxirane

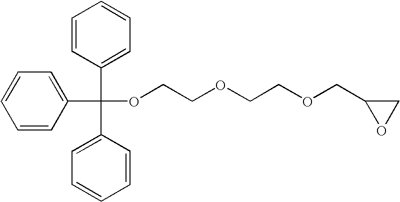 2-(2-Trityloxyethoxy)ethanol (6.65 g, 19 mmol) was dissolved in dry THF (100 mL). 60% NaH-oil suspension (0.764 mg, 19 mmol) was added slowly. The suspension was stirred for 15 min. Epibromohydrin (1.58 mL, 19 mmol) was added and the mixture was stirred under nitrogen at room temperature overnight. The reaction was quenched with ice, separated between diethyl ether (300 mL) and water (300 mL). The water phase was extracted with dichloromethane. The organic phases were collected, dried (Na₂SO₄) and solvent removed in vacou to afford an oil which was purified on silical gel column eluted with DCM/MeOH/Et₃N (98:1:1) to yield the title compound. ¹H NMR (CDCl₃): δ 7.45 (m, 6H), 7.25, (m, 9H), 3.82 (dd, 1H), 3.68 (m, 6H), 3.45 (dd, 1H), 3.25 (t, 2H), 3.15 (m, 1H), 2.78 (t, 1H), 2.59 (m, 1H). LC-MS: m/z=427 (M+Na); Rₜ=2.44 min.

### Example 22

### Example 2

### 1,3-Bis[2-(2-trityloxyethoxy)ethoxy]propan-2-ol

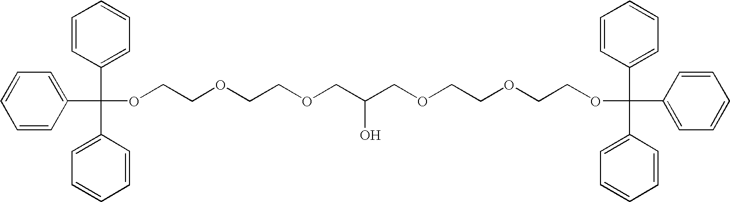

2-(2-Trityloxyethoxy)ethanol (1.14 g, 3.28 mmol) was dissolved in dry DMF (5 mL). 60% NaH-oil suspension (144 mg, 3.61 mmol) was added slowly and the mixture was stirred under nitrogen at room temperature for 30 min. The mixture was heated to 40° C. 2-[2-(2-Trityloxyethoxy)ethoxymethyl]oxirane (1.4 g, 3.28 mmol) was dissolved in dry DMF (5 mL) and added drop wise to the solution under nitrogen while stirring was maintained. After ended addition the mixture was stirred under nitrogen at 40° C. overnight. The heating was removed and after cooling to room temperature the reaction was quenched with ice and poured into saturated aqueous NaHCO₃ (100 mL). The mixture was extracted with diethyl ether (3×75 mL). The organic phases were collected, dried (Na₂SO₄), and solvent removed in vacuo to afford an oil which was purified on silical gel column eluted with EtOAc/Heptane/Et₃N (49:50:1) to yield the title compound.

¹H NMR (CDCl₃): δ 7.45 (m, 12H), 7.25, (m, 18H), 3.95 (m, 1H), 3.78-3.45 (m, 16H), 3.22 (t, 4H), LC-MS: m/z=775 (M+Na); Rₜ=2.94 min.

### Example 23

### 1,3-Bis[2-(2-trityloxyethoxy)ethoxy]propan-2-yloxy β-cyanoethyl N,N-diisopropylphosphoramidite

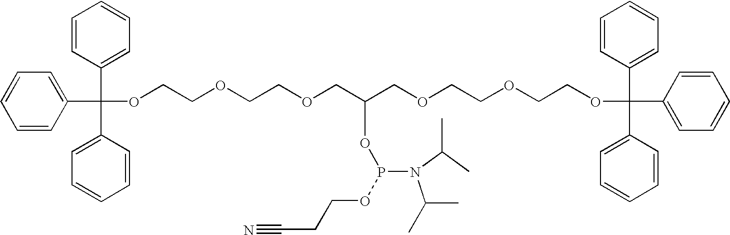

1,3-Bis[2-(2-trityloxyethoxy)ethoxy]propan-2-ol (0.95 g, 1.26 mmol) was evaporated twice from dry pyridine and once from dry acetonitrile. The residue was dissolved in dry THF (15 mL), while stirring under nitrogen. Diisopropylethylamine (1.2 mL, 6.95 mmol) was added. The mixture was coold to 0° C. with an icebath 2-cyanoethyl diisopropylchlorophosphoramidite (0.39 mL, 1.77 mmol) was added under nitrogen. The mixture was stirred for 10 minutes at 0° C. followed by 30 minutes at room temperature. Aqueous NaHCO₃ (50 mL) was added and the mixture extracted with DCM/Et₃N (98:2) (3×30 mL). The organic phases were collected, dried (Na₂SO₄), and the solvent removed in vacuo to afford an oil which was purified on silical gel column eluted with EtOAc/Heptane/Et₃N (35:60:5) to yield the 703 mg of title compound. ³¹P-NMR (CDCl₃): δ 149.6 ppm.

### Example 24

### 2-(1,3-Bis[2-(2-hydroxyethoxy)ethoxy]propan-2-yloxy) acetic acid tert-butyl ester

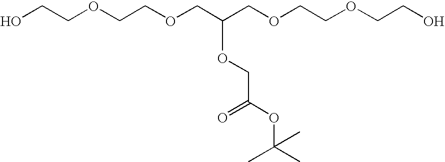

1,3-Bis[2-(2-trityloxyethoxy)ethoxy]propan-2-ol (0.3 g, 0.40 mmol) was evaporated once from dry pyridine and once from dry acetonitrile. The residual was dissolved in dry DMF (2 mL), under nitrogen, 60% NaH-oil suspension (24 mg, 0.6 mmol) was added. The mixture was stirred at room temperature for 15 minutes. tert-Butylbromoacetate (0.07 mL, 0.48 mmol) was added and the mixture was stirred for an additional 60 minutes. The reaction was quenched with ice, then partitioned between diethyl ether (100 mL) and water (100 mL). The organic phase was collected, dried (Na₂SO₄), and solvent removed in vacuo to afford an oil which was eluted on silical gel column with EtOAc/Heptane/Et₃N (49:50:1). Fraction containing main product was collected. The solvent was removed in vacuo and the residue was dissolved in 80% aqueous acetic acid (5 mL) and stirred at room temperature overnight. Solvent was removed in vacuo and the crude material dissolved in diethyl ether (25 mL), and washed with water (2×5 mL). The water phases were collected and the water removed on rotorvap to yield 63 mg of the title compound. ¹H NMR (CDCl₃): δ4.19 (s, 2H), 3.78-3.55 (m, 21H), 1.49 (s, 9H).

### Example 25

### N,N-Bis(2-(2-phthalimidoethoxy)ethyl)-O-tert-butylcarbamate

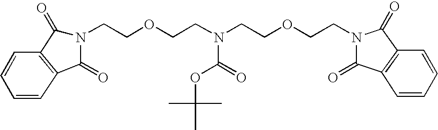

N,N-Bis(2-hydroxyethyl)-O-tert-butylcarbamate is dissolved in a polar, non-protic solvent such as THF or DMF. Sodium hydride (60% suspension in mineral oil) is added slowly to the solution. The mixture is stirred for 3 hours. N-(2-Bromoethyl)phthalimide is added. The mixture is stirred until the reaction is complete. The reaction is quenched by slow addition of methanol. Ethylacetate is added. The solution is washed with aqueous sodium hydrogencarbonate. The organic phase is dried, filtered, and subsequently concentrated under vacuum as much as possible. The crude compound is purified by standard column chromatography.

### Example 26

### N,N-Bis(2-(2-aminoethoxy)ethyl)-O-tert-butylcarbamate

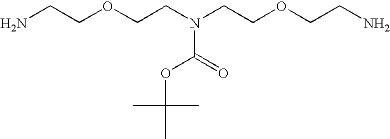

N,N-Bis(2-(2-phthalimidoethoxy)ethyl)-O-tert-butylcarbamate is dissolved in a polar solvent such as ethanol. Hydrazine (or another agent known to remove the phthaloyl protecting group) is added. The mixture is stirred at room temperature (or if necessary elevated temperature) until the reaction is complete. The mixture is concentrated under vacuum as much as possible. The crude compound is purified by standard column chromatography or if possible by vacuum destillation.

### Example 27

### N,N-Bis(2-(2-benzyloxycarbonylaminoethoxy)ethyl)-O-tert-butylcarbamate

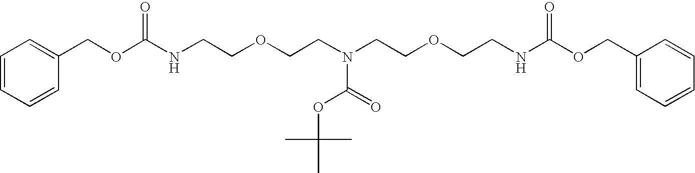

N,N-Bis(2-(2-aminoethoxy)ethyl)-O-tert-butylcarbamate is dissolved in a mixture of aqueous sodium hydroxide and THF or in a mixture of aqueous sodium hydroxide and acetonitrile. Benzyloxychloroformate is added. The mixture is stirred at room temperature until the reaction is complete. If necessary, the volume is reduced in vacuo. Ethyl acetate is added. The organic phase is washed with brine. The organic phase is dried, filtered, and subsequently concentrated in vacuo as much as possible. The crude compound is purified by standard column chromatography.

### Example 28

### Bis(2-(2-phthalimidoethoxy)ethyl)amine

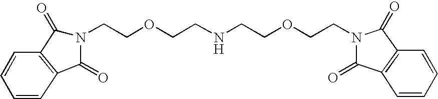

Bis(2-(2-phthalimidoethoxy)ethyl)-tert-butylcarbamate is dissolved in trifluoroacetic acid. The mixture is stirred at room temperature until the reaction is complete. The mixture is concentrated in vacuo as much as possible. The crude compound is purified by standard column chromatography.

### Example 29

### 11-Oxo-17-phthalimido-12-(2-(2-phthalimidoethoxy)ethyl)-3,6,9,15-tetraoxa-12-azaheptadecanoic acid

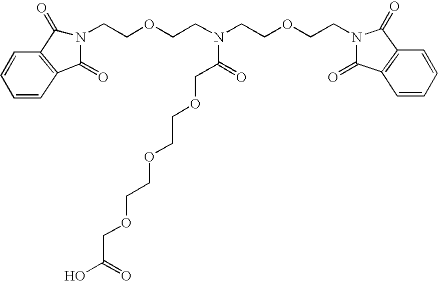

3,6,9-Trioxaundecanoic acid is dissolved in dichloromethane. A carbodiimide (e.g., N,N-dicyclohexylcarbodiimide or N,N-diisopropylcarbodiimide) is added. The solution is stirred over night at room temperature. The mixture is filtered. The filtrate can be concentrated in vacuo if necessary. The acylation of amines with the formed intramolecular anhydride is known from literature (e.g., Cook, R. M.; Adams, J. H.; Hudson, D. Tetrahedron Lett., 1994, 35, 6777-6780 or Stora, T.; Dienes, Z.; Vogel, H.; Duschl, C. Langmuir 2000, 16, 5471-5478). The anhydride is mixed with a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 30

### 5-Oxo-11-phthalimido-6-(2-(2-phthalimidoethoxy)ethyl)-3,9-dioxa-6-azaundecanoic acid

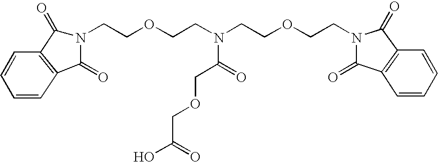

A solution of diglycolic anhydride in a non-protic solvent such as dichloromethane or N,N-dimethylformamide is added dropwise to a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 31

### 1,2,3-Benzotriazin-4(3H)-one-3-yl 2-[2-(2-methoxyethoxy)ethoxy]acetate

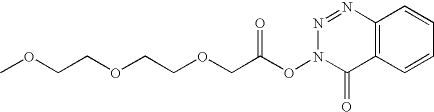

3-Hydroxy-1,2,3-benzotriazin-4(3H)-one (10.0 g; 61.3 mmol) and 2-[2-(2-methoxyethoxy)ethoxy]acetic acid (10.9 g; 61.3 mmol) was suspended in DCM (125 ml) and DIC (7.7 g; 61.3 mmol) was added. The mixture was stirred under a dry atmosphere at ambient temperature over night. A precipitate of diisopropyl urea was formed, which was filtered off. The organic solution was washed extensively with aqueous saturated sodium hydrogen carbonate solution, then dried (Na₂SO₄) and evaporated in vacuo, to give the title product as a clear yellow oil. Yield was 16.15 g (81%). ¹H-NMR (CDCl₃): δ 3.39 ppm (s, 3H); 3.58 (t, 2H); 3.68 (t, 2H); 3.76 (t, 2H); 3.89 (t, 2H); 4.70 (s, 2H); 7.87 (t, 1H); 8.03 (t, 1H); 8.23 (d, 1H); 8.37 (d, 1H). ¹³C-NMR (CDCl₃, selected peaks): δ 57.16 ppm; 64.96; 68.71; 68.79; 69.59; 69.99; 120.32; 123.87; 127.17; 130.96; 133.63; 142.40; 148.22; 164.97.

Oligomer Products

Solid Phase Oligomerisation:

The reactions described below are all performed on polystyrene functionalised with the Wang linker. The reactions will in general also work on other types of solid supports, as well as with other types of functionalised linkers.

Solid Phase Azide Reduction:

The reaction is known (Schneider, S. E. et al. Tetrahedron, 1998, 54(50) 15063-15086) and can be performed by treating the support bound azide with excess of triphenyl phosphine in a mixture of THF and water for 12-24 hours at room temperature. Alternatively, trimethylphosphine in aqueous THF as described by Chan, T. Y. et al Tetrahedron Lett. 1997, 38(16), 2821-2824 can be used. Reduction of azides can also be performed on solid phase using sulfides such as dithiothreitol (Meldal, M. et al. Tetrahedron Lett. 1997, 38(14), 2531-2534) 1,2-dimercaptoethan and 1,3-dimercaptopropan (Meinjohanns, E. et al. J. Chem. Soc, Perkin Trans 1, 1997, 6, 871-884) or tin(II) salts such as tin(II)chloride (Kim, J. M. et al. Tetrahedron Lett, 1996, 37(30), 5305-5308).

Solid Phase Carbamate Formation:

The reaction is known and is usually performed by reacting an activated carbonate, or a halo formiate derivative with an amine, preferable in the presence of a base.

### Example 32

### 3-(1,3-Bis{2-[2-(1,3-Bis[2-(2-{2-[2-(2-methoxyethoxy)ethoxy]acetamino}ethoxy)-ethoxy]propan-2-yloxy)acetylamino]ethoxy])ethoxy}propan-2-yloxy)acetylamino)-propanoicacid

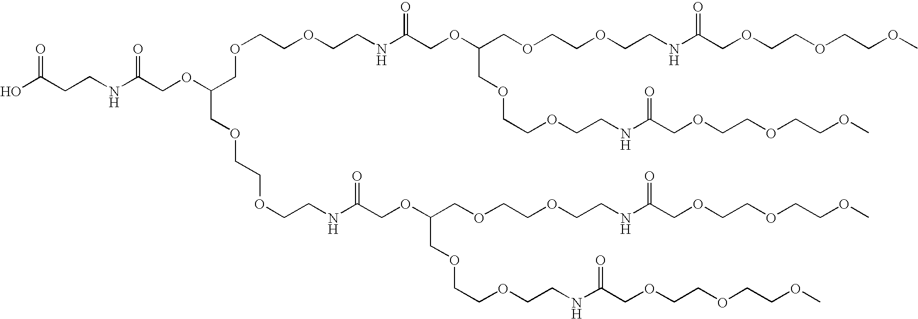

This example uses the 2-(1,3-Bis[azidoethoxyethyl]propan-2-yloxy)acetic acid monomer building block prepared in example 6 in the synthesis of a second generation amide based branched polymer capped with 2-[2-(2-methoxyethoxy)ethoxy]acetic acid. The coupling chemistry is based on standard solid phase peptide chemistry, and the protection methodology is based on a solid phase azide reduction step as described above.

Step 1: Fmoc-βala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 2-(1,3-bis[azidoethoxyethyl]propan-2-yloxy)acetic acid (527 mg; 1,4 mmol, 4×) and DhbtOH (225 mg; 1,4 mmol, 4×) were dissolved in DMF (5 ml) and DIC (216 ul, 1,4 mmol, 4×) was added. The mixture was left for 10 min (pre-activation) then added to the resin together with DIPEA (240 ul; 1,4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was redrawn and treated with a solution of benzoylchloride (0.5 M) and DIPEA (1 M) in DMF for 1 h. The resin was cleaved with 50% TFA/DCM and the dibenzoylated product analysed with NMR and LC-MS. ¹H-NMR (CDCl₃): 3.50-3.75 (m, 20H); 3.85 (s, 1H); 4.25 (d, 2H); 6.95 (t, 1H); 7.40-7.50 (m, 6H); 7.75 (m, 4H). LC-MS (any-one): m/e=576 (M+1)⁺; Rₜ=2.63 min.

Step 5-7 was performed as step 2-4 using a double molar amount of reagents but same amount of solvent.

Step 8: capping with 2-[2-(2-methoxyethoxy)ethoxy]acetic acid: A solution of 2-[2-(2-methoxyethoxy)ethoxy]acetic acid (997 mg; 5.6 mmol, 16× with respect to resin loading) and DhbtOH (900 mg; 5.6 mmol, 16×) are dissolved in DMF (5 ml) and DIC (864 ul, 5.6 mmol, 16×) is added. The mixture is left for 10 min (pre-activation) then added to the resin together with DIPEA (960 ul; 5.6 mmol, 16×). The resin is shaken for 90 min, then drained and washed with DMF (3×) and DCM (3×).

Step 9: Cleavage from resin: The resin is treated with a 50% TFA-DCM solution at ambient temperature for 30 min. The solvent is collected and the resin is washed an additional time with 50% TFA-DCM. The combined filtrates are evaporated to dryness, and the residue purified by chromatography.

### Example 33

### 3-(1,3-Bis{2-[2-(1,3-bis[2-(2-{2-[2-(2-methoxyethoxy)ethoxy]acetamino}ethoxy)ethoxy]-propan-2-yloxycarbonyl)amino]ethoxy])ethoxy}propan-2-yloxycarbonyl)amino)-propanoicacid

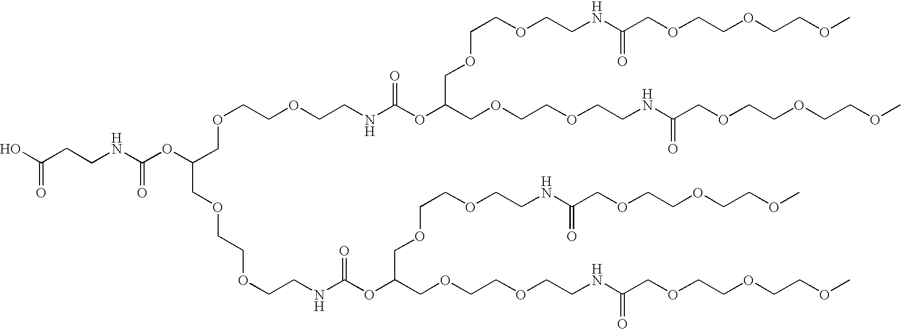

This example uses the 1,3-Bis[2-(2-azidoethoxy)ethoxy]porpan-2-yl-p-nitrophenylcarbonate monomer building block prepared in example 4 in the synthesis of a second generation carbamate based branched polymer capped with 2-[2-(2-methoxyethoxy)ethoxy]acetic acid. The coupling chemistry is based on standard solid phase carbamate chemistry, and the protection methodology is based on a solid phase azide reduction step as described above.

Step 1: Fmoc-βala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 1,3-Bis[azidoethoxyethyl]propan-2-yl-p-nitrophenylcarbamate (527 mg; 1,4 mmol, 4×). was added to the resin together with DIPEA (240 ul; 1,4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was redrawn and treated with a solution of benzoylchloride (0.5 M) and DIPEA (1 M) in DMF for 1 h. The resin was cleaved with 50% TFA/DCM and the dibenzoylated product analysed with NMR and LC-MS. ¹H-NMR (CDCl₃): 3.50-3.75 (m, 20H); 3.85 (s, 1H); 4.25 (d, 2H); 6.95 (t, 1H); 7.40-7.50 (m, 6H); 7.75 (m, 4H). LC-MS (any-one): m/e=576 (M+1)⁺; Rₜ=2.63 min.

Step 5-7 was performed as step 2-4 using a double molar amount of reagents but same amount of solvent.

Step 8: capping with 2-[2-(2-methoxyethoxy)ethoxy]acetic acid: A solution of 2-[2-(2-methoxyethoxy)ethoxy]acetic acid (997 mg; 5.6 mmol, 16× with respect to resin loading) and DhbtOH (900 mg; 5.6 mmol, 16×) are dissolved in DMF (5 ml) and DIC (864 ul, 5.6 mmol, 16×) is added. The mixture is left for 10 min (pre-activation) then added to the resin together with DIPEA (960 ul; 5.6 mmol, 16×). The resin is shaken for 90 min, then drained and washed with DMF (3×) and DCM (3×).

Step 9: Cleavage from resin: The resin is treated with a 50% TFA-DCM solution at ambient temperature for 30 min. The solvent is collected and the resin is washed an additional time with 50% TFA-DCM. The combined filtrates are evaporated to dryness, and the residue purified by chromatography.

### Example 34

### 3-[2-(1,3-Bis[2-(2-{2-[2-(2-methoxyethoxy)ethoxy]acetylamino}ethoxy)ethoxy]propan-2-yloxy)acetylamino]propanoic acid

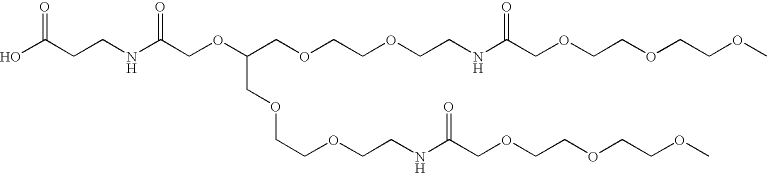

Step 1: Fmoc-β-alanine linked Wang resin (A22608, Nova Biochem, 3.00 g; with loading 0.83 mmol/g) was svelled in DCM for 20 min. then washed with DCM (2×20 ml) and NMP (2×20 ml). The resin was then treated twice with 20% piperidine in NMP (2×15 min). The resin was washed with NMP (3×20 ml) and DCM (3×20 ml).

Step 2: 2-(1,3-Bis[2-(2-azidoethoxy)ethoxy]propan-2-yloxy)acetic acid (3.70 g; 10 mmol) was dissolved in NMP (30 ml) and DhbtOH (1.60 g; 10 mmol) and DIC (1.55 ml; 10 mmol) was added. The mixture was stirred at ambient temperature for 30 min, then added to the resin obtained in step 1 together with DIPEA (1.71 ml; 10 mmol). The reaction mixture was shaken for 1.5 h, then drained and washed with NMP (5×20 ml) and DCM (3×20 ml).

Step 3: A solution of SnCl₂.2H₂O (11.2 g; 49.8 mmol) in NMP (15 ml) and DCM (15 ml) was then added. The reaction mixture was shaken for 1 h. The resin was drained and washed with NMP:MeOH (5×20 ml; 1:1). The resin was then dried in vacuo.

Step 4: A solution of 2-[2-(2-methoxyethyl)ethoxy]acetic acid (1.20 g; 6.64 mmol), DhbtOH (1.06 g; 6.60 mmol) and DIC (1.05 ml; 6.60 mmol) in NMP (10 ml) was mixed for 10 min, at room temperature, and then added to the 3-[2-(1,3-bis[2-(2-aminoethoxy)ethoxy]propan-2-yloxy)acetylamino]propanoic acid tethered wang resin (1.0 g; 0.83 mmol/g) obtained in step 3. DIPEA (1.15 ml, 6.60 mmol) was added, and the reaction mixture was shaken for 2.5 h. Solvent was removed, and the resin was washed with NMP (5×20 ml) and DCM (10×20 ml).

Step 5: The resin product of step 4 was treated with TFA:DCM (10 ml, 1:1) for 1 hour. The resin was filtered and washed once with TFA:DCM (10 ml, 1:1). The combined filtrate and washing was then taken dryness, to give a yellow oil (711 mg). The oil was dissolved in 10% acetonitril-water (20 ml), and purified over two runs on a preparative HPLC apparatus using a C18 column, and a gradient of 15-40% acetonitril-water. Fractions were subsequently analysed by LC-MS. Fractions containing product were pooled and taken to dryness. Yield: 222 mg (37%). LC-MS: m/z=716 (m+1), Rₜ=1.97 min. ¹H-NMR (CDCl₃): δ 2.56 ppm (t, 2H); 3.36 (s, 6H); 3.46-3.66 (m, 39H); 4.03 (s, 4H); 4.16 (s, 2H); 7.55 (t, 2H); 8.05 (t, 1H). ¹³C-NMR (CDCl₃, selected peaks): δ 33.71 ppm; 34.90; 58.89; 68.94; 69.40; 69.98; 70.09; 70.33; 70.74; 70.91; 71.07; 71.74; 79.07; 171.62; 171.97; 173.63.

### Example 35

### 3-(1,3-Bis{2-(2-[2-(1,3-bis[2-(2-{2-[2-(2-methoxyethoxy)ethoxy]acetamino}ethoxy)-ethoxy]propan-2-yloxy)acetylamino]ethoxy)ethoxy}propan-2-yloxy)acetylamino)propanoic acid

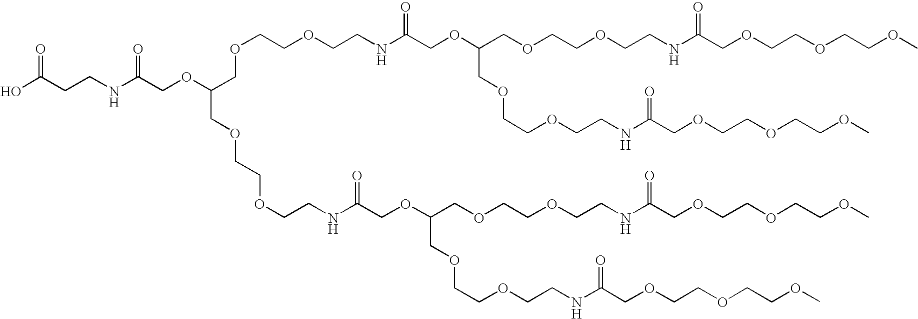

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 34 by repeating step 2-5, doubling the amount of reagents used.

Yield: 460 mg (33%). MALDI-MS (α-cyanohydroxycinnapinic acid matrix): m/z=1670 (M+Na⁺). ¹H-NMR (CDCl₃): δ 2.57 ppm (t, 2H); 3.38 (s, 12H); 3.50-3.73 (m, 85H); 4.05 (s, 8H); 4.17 (s, 2H); 4.19 (s, 4H); 7.48 (m, 4H); 7.97 (m, 3H). ¹³C-NMR (CDCl₃, selected peaks): δ 38.81 ppm; 58.92; 69.46; 69.92; 70.05; 70.05; 70.13; 70.40; 70.73; 70.97; 71.11; 71.88; 76.74; 77.06; 77.38; 171.33; 172.02.

### Example 36

### 3-(1,3-Bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoic acid

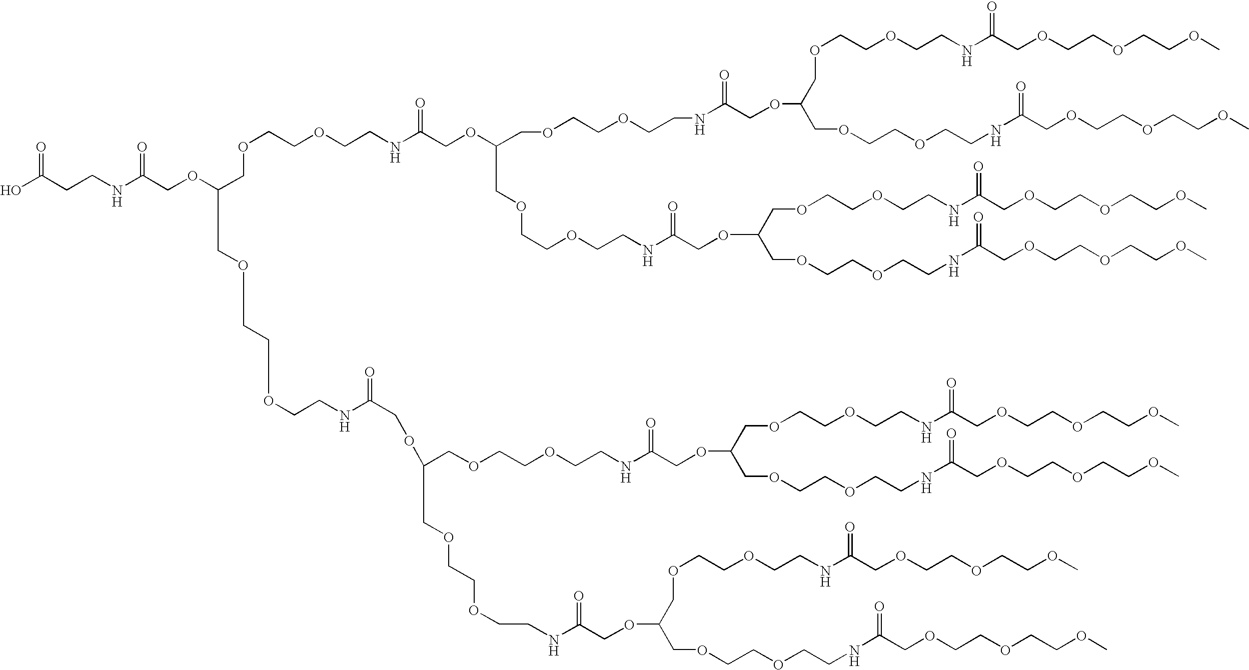

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 34 by repeating step 2-3 with 2× the amount of reagents used, then repeating step 2-5 with 4× the amount of reagent used. Yield: 84 mg (4%). LC-MS: (m/2)+1=1758; (m/3)+1=1172; (m/4)+1=879; (m/5)+1=704. Rₜ=2.72 min. ¹H-NMR (CDCl₃): δ 2.51 ppm (t, 2H); 3.33 (s, 24H); 3.44-3.70 (m, 213H); 3.93 (s, 16H); 4.08 (s, 14H); 7.25 (m, 8H); 7.69 (m, 7H). ¹³C-NMR (CDCl₃, selected peaks): δ 38.94 ppm; 59.33; 69.78; 70.08; 70.37; 70.44; 70.56; 70.82; 71.10; 71.26; 71.51; 72.17; 79.24; 170.60; 171.22.

### Example 37

### N-Hydroxysuccinimidyl 3-[2-(1,3-bis[2-(2-{2-[2-(2-methoxyethoxy)ethoxy]-acetylamino}ethoxy)ethoxy]propan-2-yloxy)acetylamino]propanoate

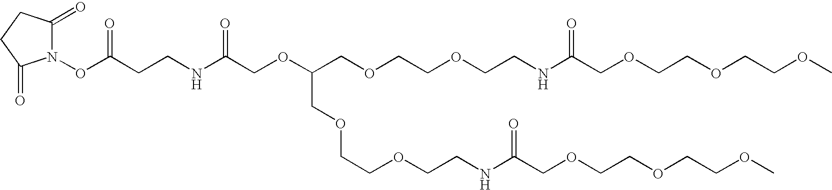

3-[2-(1,3-Bis[2-(2-{2-[2-(2-methoxyethoxy)ethoxy]acetylamino}ethoxy)ethoxy]propan-2-yloxy)acetylamino]propanoic acid (67 mg; 82 mmol) was dissolved in THF (5 ml). The reaction mixture was cooled on an icebath. DIPEA (20 ul; 120 mmol) and TSTU (34 mg; 120 mmol) was added. The mixture was stirred at ambient temperature overnight at which time, the reaction was complete according to LC-MS. LC-MS: m/z=813 (M+H)⁺; Rₜ=2.22 min.

### Example 38

### N-Hydroxysuccinimidyl 3-(1,3-bis{2-(2-[2-(1,3-bis[2-(2-{2-[2-(2-methoxyethoxy)-ethoxy]acetamino}ethoxy)ethoxy]propan-2-yloxy)acetylamino]ethoxy)ethoxy}propan-2-yloxy)acetylamino)propanoate

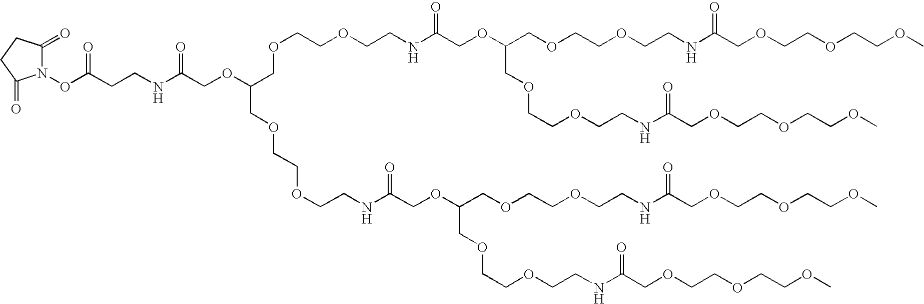

Prepared from 3-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)propanoic acid and TSTU as described in example 37. LC-MS: (m/2)+1=873, Rₜ=2.55 min.

### Example 39

### N-Hydroxysuccimidyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]-ethoxy)ethoxy}propan-2-yloxy)acetylamino)ethoxy)ethoxy}propan-2-yloxy)-acetylamino)propanoate

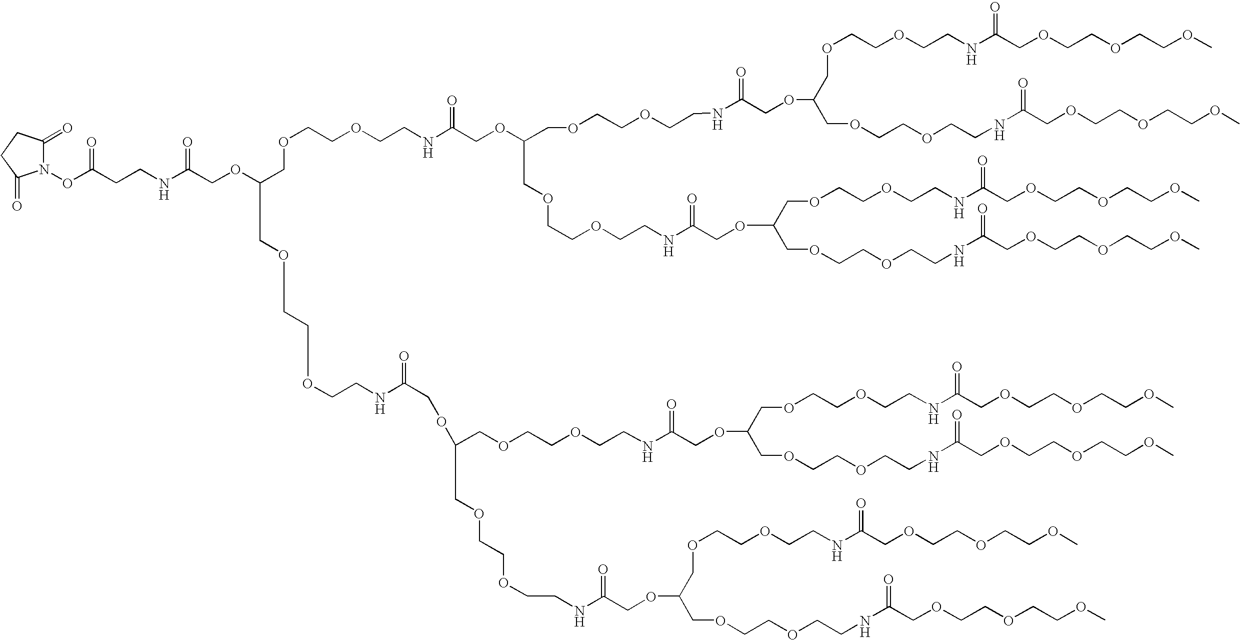

Prepared from N-hydroxysuccinimidyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}-propan-2-yloxy)acetylamino)ethoxy)ethoxy}propan-2-yloxy)acetylamino)propanoic acid and TSTU as described in example 37. LC-MS: (m/4)+1=903, Rₜ=2.69 min.

### Example 40

### N-(4-tert-Butoxycarbonylaminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

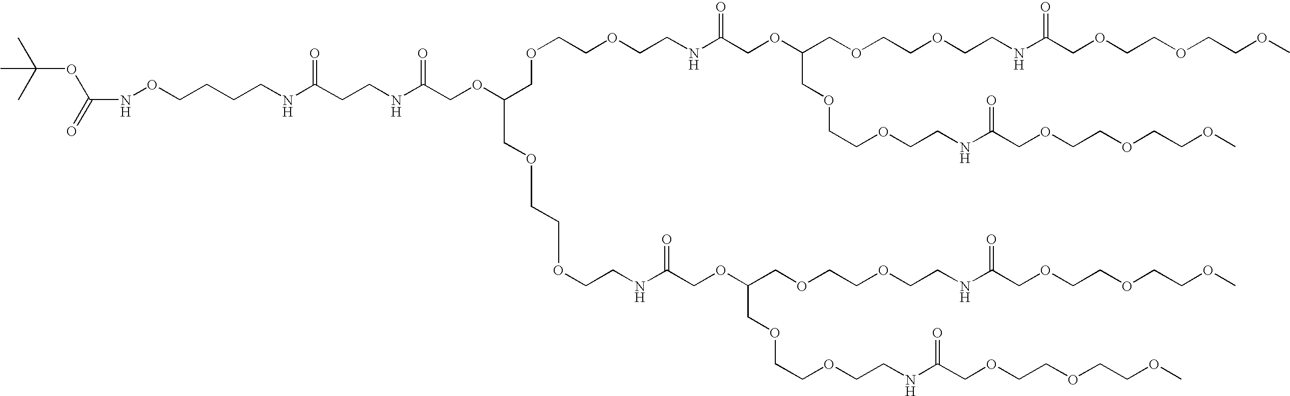

N-Hydroxysuccinimidyl 3-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)propanoate (105 mg; 0.06 mmol) was dissolved in DCM (2 ml). Then a solution of 4-(tert-butyloxycarbonylaminoxy)butylamine (49 mg; 0.24 mmol) was added followed by DIPEA (13 ul; 0.07 mmol). The mixture was stirred at ambinet temperature for one hour, then concentrated under reduced presure. The residual was dissolved in 20% acetonitril-water (4 ml), and purified on a preparative HPLC apparatus using a C18 column, and a step gradient of 0, 10, 20, 30, and 40% (10 ml elutions each) of acetonitril-water. Fractions containing pure product was concentrated and dried for 16 h in a vacuum oven to give a yellow oil. Yield: 57 mg (51%). LC-MS: (m/2)+1=918, Rₜ=2.75 min. ¹H-NMR (CDCl₃): δ 1.42 ppm (s, 9H); 2.40 (t, 2H); 3.21 (dd, 2H); 3.33 (s, 12H); 3.38-3.72 (m, 99H); 3.80 (m, 2H); 3.95 (s, 8H); 4.08 (s, 6H); 6.99 (m, 1H); 7.23 (m, 4H); 7.69 (m, 2H); 7.85 (m, 1H); 8.00 (m, 1H). ¹³C-NMR (CDCl₃, selected peaks): δ 28.27 ppm; 38.58; 58.97; 69.42; 69.72; 70.01; 70.08; 70.20; 70.41; 70.46; 70.73; 70.91; 71.16; 71.22; 71.81; 78.89; 81.33; 170.27; 170.89.

### Example 41

### N-(4-Aminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

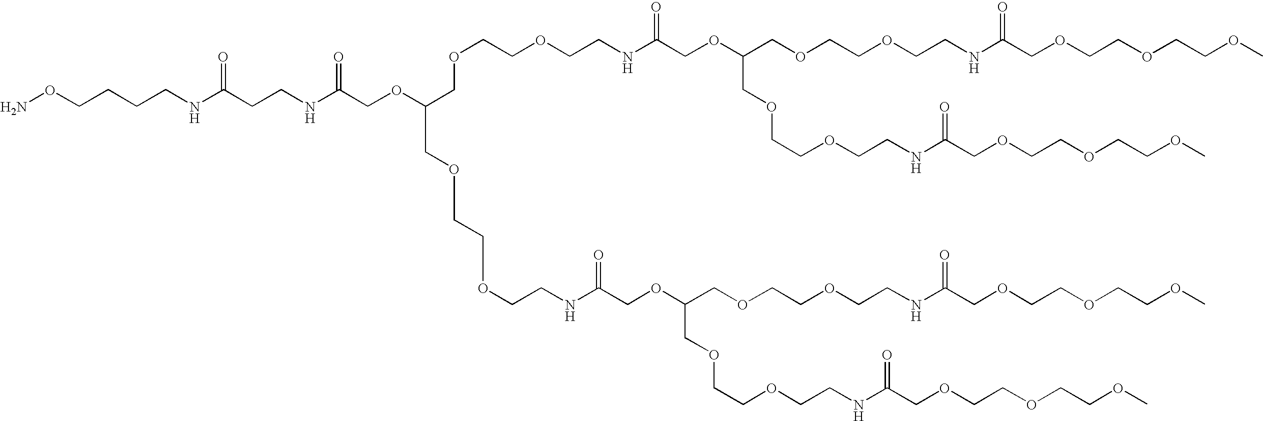

N-(4-tert-Butoxycarbonylaminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide (19 mg; 10 mmol) was dissolved in 50% TFA/DCM (10 ml), and the clear solution was stirred at ambient temperature for 30 min. The solvent was removed by rotaryevaporation, and the residue was stripped twice from DCM, to give a quantitative yield (19 mg) of the title product. LC-MS: (m/2)+1=868, (m/3)+1=579, Rₜ=2.35 min.

### Example 42

### t-Butyl 2-(1,3-bis[2-(2-{2-[2-(2-methoxyethoxy)ethoxy]acetamino}ethoxy)-ethoxy]propan-2-yloxy)acetate

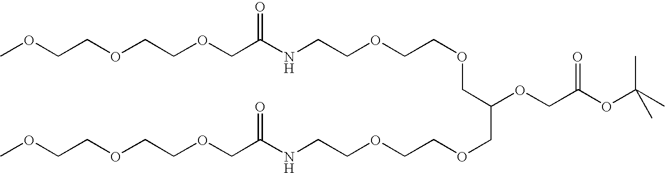

t-Butyl 2-(1,3-bis[2-(2-aminoethoxy)ethoxy]propan-2-yloxy)acetate (1.74 g; 4.5 mmol) and 1,2,3-benzotriazin-4(3H)-one-3-yl 2-[2-(2-methoxyethoxy)ethoxy]acetate (2.94 g; 9 mmol) was dissolved in DCM (100 ml). DIPEA (3.85 ml; 22.3 mmol) was added and the celar mixture was stirred for 90 min at room temperature. Solvent was removed in vacuo, and the residue was purified by chromatography on silica, using MeOH-DCM (1:16) as eluent. Pure fractions were pooled and taken to dryness to give the title material as a clear oil. Yield was 1.13 g (36%). ¹H-NMR (CDCl₃): δ 1.46 ppm (s, 9H); 3.38 (s, 6H); 3.49-3.69 (m, 37H); 4.01 (s, 4H); 4.18 (s, 2H); 7.20 (bs, 2H).

### Example 43

### 2-(1,3-Bis[2-(2-{2-[2-(2-methoxyethoxy)ethoxy]acetamino}ethoxy)ethoxy]propan-2-yloxy)acetic acid

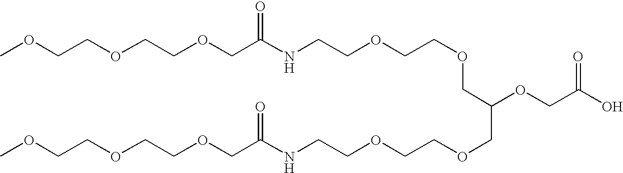

t-Butyl 2-(1,3-bis[2-(2-aminoethoxy)ethoxy]propan-2-yloxy)acetate (470 mg; 0.73 mmol) was dissolved in DCM-TFA (25 ml, 1:1) and the mixture was stirred for 30 min at ambient temperature. The solvent was removed, in vacuo, and the residue was stripped twice from DCM. LC-MS: (m+1)=645, Rₜ=2.26 min. ¹H-NMR (CDCl₃): δ 3.45 ppm (s, 6H); 3.54-3.72 (m, 37H); 4.15 (s, 4H); 4.36 (s, 2H).

### Example 44

### N-Hydroxysuccimidyl 2-(1,3-bis[2-(2-{2-[2-(2-methoxyethoxy)ethoxy]acetamino}ethoxy)ethoxy]propan-2-yloxy)acetate

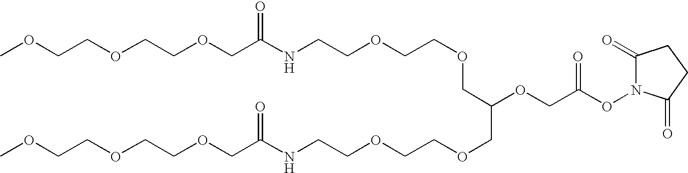

2-(1,3-Bis[2-(2-{2-[2-(2-methoxyethoxy)ethoxy]acetamino}ethoxy)ethoxy]propan-2-yloxy)acetic acid (115 mg; 0.18 mmol) was dissolved in THF (5 ml). The reaction mixture was placed on an ice bath. TSTU (65 mg, 0.21 mmol) and DIPEA (37 ul; 0.21 mmol) was added and the reaction mixture was stirred at 0° C. for 30 min, then at room temperature overnight. The reaction was then taken to dryness, to give 130 mg of the title material as an clear oil. LC-MS: (m+1)=743, (m/2)+1=372, Rₜ=2.27 min.

### Example 45

### t-Butyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetate

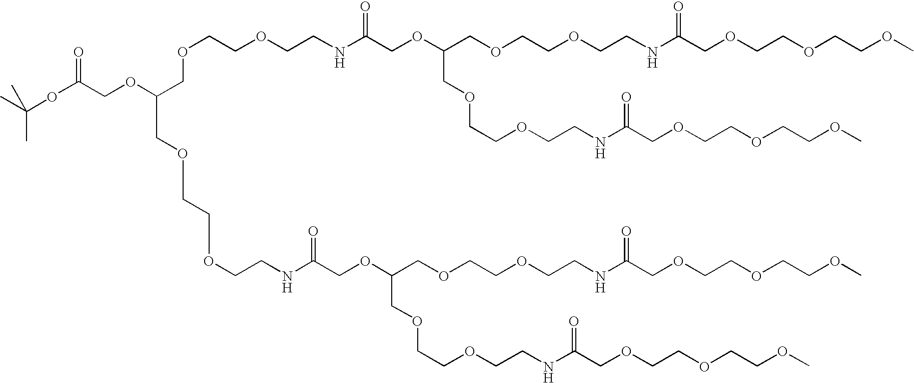

The material is prepared from two equivalents of N-hydroxysuccimidyl 2-(1,3-bis[2-(2-{2-[2-(2-methoxyethoxy)ethoxy]acetamino}ethoxy)ethoxy]propan-2-yloxy)acetate and one equivalent of t-Butyl 2-(1,3-bis[2-(2-aminoethoxy)ethoxy]propan-2-yloxy)acetate, using the protocol and purification method described in example 42. Subsequent removal of t-butyl group is done as described in example 43 and N-hydroxysuccimidyl ester formation is done as described in example 44.

### Example 46

### (S)-2,6-Bis-(2-[2-(2-[2-(2,6-bis-[2-(2-[2-(2-azidoethoxy)ethoxy]ethoxy)acetylamino]hexanoylamino)ethoxy]ethoxy)ethoxy]acetylamino)hexanoic acid methyl ester

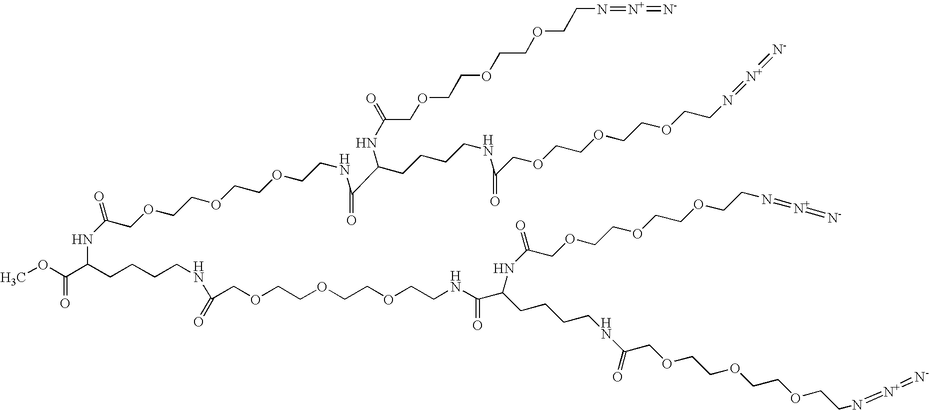

(S)-2,6-Bis-(2-{2-[2-(2-azidoethoxy)ethoxy]ethoxy}acetylamino)hexanoic acid (1.8 g, 3.10 mmol)) was dissolved in a mixture of dimethylformamide/dichloromethane 1:3 (10 ml), pH was adjusted to basic reaction using diisopropylethylamine, N-hydroxybenzotriazole and 1-ethyl-3-(3-dimethylaminopropylcarbodiimide hydrochloride were added and the reaction mixture was standing for 30 min. Then this reaction mixture was added to a solution of (S)-2,6-bis-(2-{2-[2-(2-aminoethoxy)ethoxy]ethoxy}acetylamino)hexanoic acid methyl ester (0.37 g, 0.70 mmol) in dichloromethane) and the reaction mixture was standing night over.

The reaction mixture was diluted with dichloromethane (150 ml), washed with water (2×40 ml), 50% saturated sodiumhydrogencarbonate (2×30 ml) and water (3×40 ml). The organic phase was dried over magnesium sulphate, filtered and evaporated in vacuo giving an oil. Yield: 1.6 g (89%). LC-MS: m/z=1656 (M+1) and m/z=828.8 (M/2)+1 and m/z=553(M/3)+1.

### Example 47

### (S)-2,6-Bis-(2-[2-(2-[2-((S)-2,6-bis-[2-(2-[2-(2-tert-butoxycarbonylaminoethoxy)ethoxy]ethoxy)acetylamino]hexanoylamino)ethoxy]ethoxy)ethoxy]acetylamino)hexanoic acid methyl ester

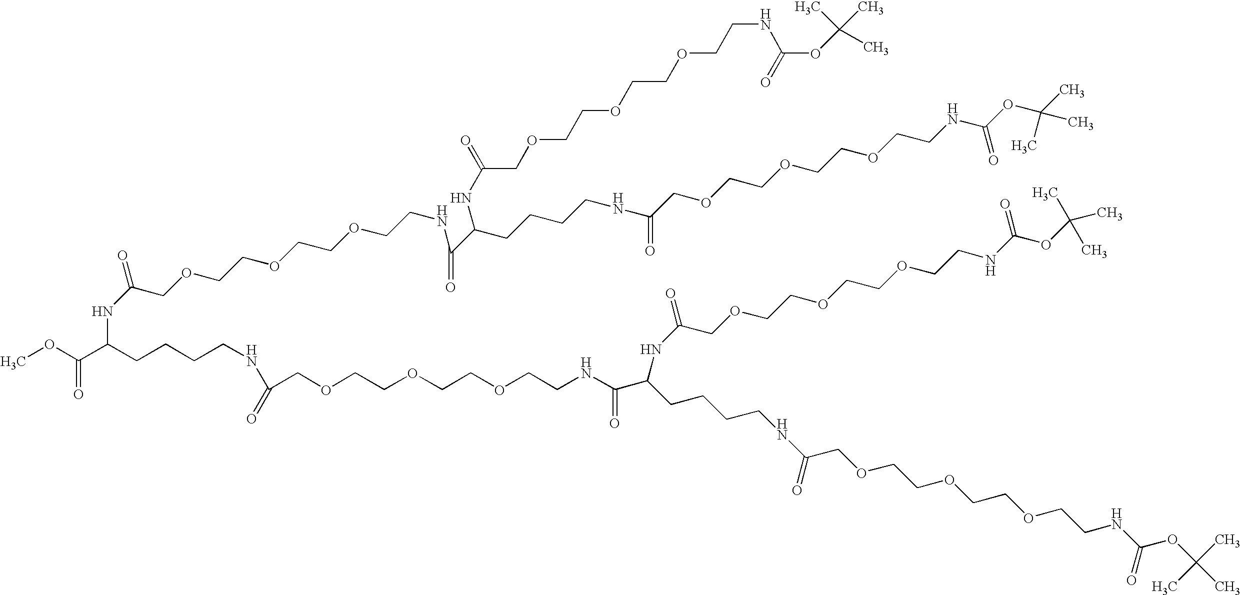

To a solution of the above (S)-2,6-Bis-(2-[2-(2-[2-((S)-2,6-bis-[2-(2-[2-(2-azidoethoxy)ethoxy]ethoxy)acetylamino]hexanoylamino)ethoxy]ethoxy)ethoxy]acetylamino)hexanoic acid methyl ester (1.6 g, 0.97 mmol) in ethylacetate (60 ml), was added di-tert-butyl dicarbonate (1.0 g, 4.8 mmol) and Pd/C (10%, 1.1 g). Hydrogen was constantly bubbled through the reaction mixture for 2 hours. The reaction mixture was filtered and the organic solvent was removed in vacuo giving an oil which was used without further purification. Yield: 1.8 g (98%). LC-MS: m/z=1953 (M+1) and m/z=977 (M/2)+1.

### Example 48

### (S)-2,6-Bis-(2-[2-(2-[2-((S)-2,6-bis-[2-(2-[2(2aminoethoxy)ethoxy]ethoxy)acetylamino]hexanoylamino)ethoxy]ethoxy)ethoxy]acetylamino)hexanoic acid methyl ester

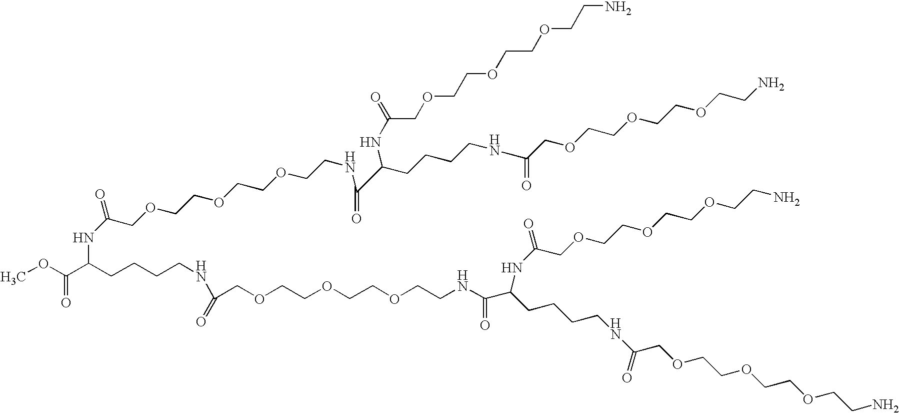

The above (S)-2,6-bis-(2-[2-(2-[2-((S)-2,6-bis-[2-(2-[2-(2-tert-butoxycarbonylamino-ethoxy)ethoxy]ethoxy)acetylamino]hexanoylamino)ethoxy]ethoxy)ethoxy]-acetylamino)hexanoic acid methyl ester was dissolved in dichloromethane (20 ml) and trifluoroacetic acid (20 ml) was added. The reaction mixture was standing for 2 hours. The organic solvent was evaporated in vacuo, giving an oil.

Yield: 1.4 g (100%). LC-MS: m/z=1552 (M+1); 777.3 (M/2)+1; 518.5 (M/3)+1 and 389.1 (M/4)+1.

### Example 49

### (S)-2,6-Bis-(2-[2-(2-[2-((S)-2,6-bis-[2-(2-[2-(2-(2-(2-(2-methoxyethoxy)ethoxy)acetylamino)ethoxy)ethoxy]ethoxy)acetylamino]hexanoylamino)ethoxy]ethoxy)ethoxy]acetylamino)hexanoic acid methyl ester

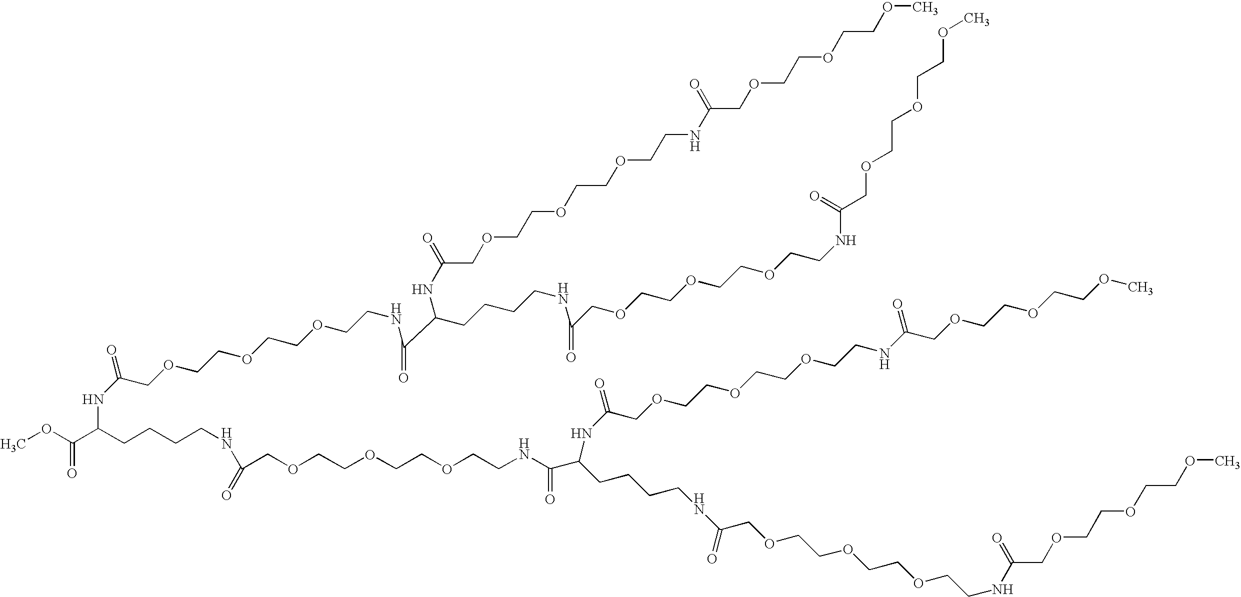

To a solution of 2-(2-(methoxyethoxy)ethoxy)acetic acid (1.3 g, 7.32 mmol) in a mixture of dichloromethane and dimethylformamide 3:1 (20 ml) was added N-hydroxysuccinimide (0.8 g, 7.32 mmol) and 1-ethyl-3-(3-dimethylaminopropylcarbodiimide hydrochloride (1.4 g, 7.32 mmol). The reaction mixture was standing for 1 hour, where after the mixture was added to a solution of (S)-2,6-bis-(2-[2-(2-[2-((S)-2,6-bis-[2-(2-[2(2aminoethoxy)ethoxy]-ethoxy)acetylamino]hexanoylamino)ethoxy]ethoxy)ethoxy]acetylamino)hexanoic acid methyl ester (1.42 g, 0.92 mmol) and diisopropylethylamine (2.4 ml, 14.64 mmol) in dichloromethane (10 ml). The reaction mixture was standing night over. The reaction mixture was diluted with dichloromethane (100 ml) and extracted with water (3×25 ml). The combine water-phases were extracted with additional dichloromethane (2×75 ml). The combined organic phases were dried over magnesium sulphate filtered and evaporated in vacuo. The residue was purified by flash chromatography using 500 ml ethyl acetate, followed by 500 ml ethyl acetate/methanol 9:1 and finally methanol as the eluent. Fractions containing product were evaporated in vacuo giving an oil. Yield: 0.75 g (38%). LC-MS: m/z=1097 (M/2)+1; 732 (M/3)+1 and 549 (M/4)+1.

The (S)-2,6-Bis-(2-[2-(2-[2-((S)-2,6-bis-[2-(2-[2-(2-(2-(2-(2-methoxyethoxy)ethoxy)acetylamino)ethoxy)ethoxy]ethoxy)acetylamino]hexanoylamino)ethoxy]ethoxy)ethoxy]acetylamino)hexanoic acid methyl ester can be saponified to the free acid and attached to an amino group of a peptide or protein using via an activated ester. The activated ester may be produced and coupled to the amino group of the peptide or protein by standard coupling methods known in the art such as diisopropylethylamine and N-hydroxybenzotriazole or other activating conditions.

### Example 50

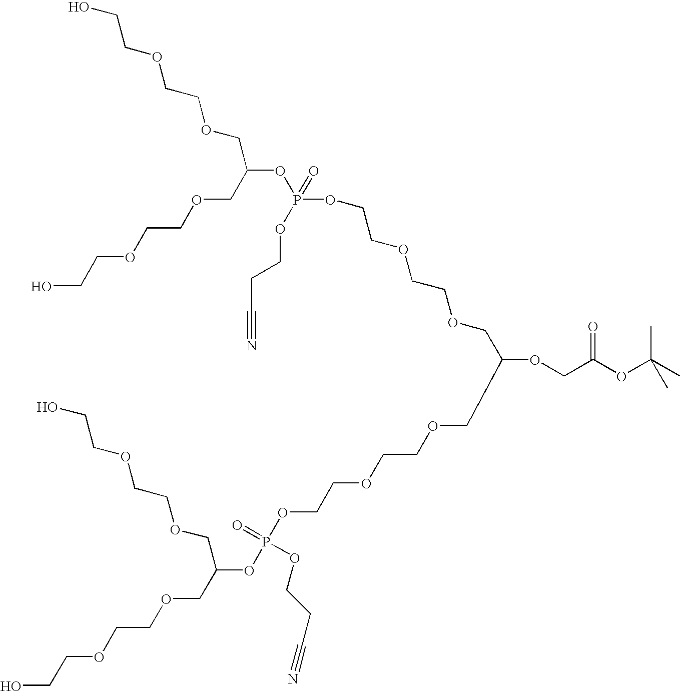

2-(1,3-Bis[2-(2-hydroxyethoxy)ethoxy]propan-2-oxy)acetic acid tert-butyl ester (63 mg, 0.16 mmol) was evaporated twice from dry acetonitrile. 1,3-Bis[2-(2-trityloxyethoxy)ethoxy]propan-2-oxy β-cyanoethyl N,N-diisopropylphosphoramidite (353 mg, 0.37 mmol) was evaporated twice from dry acetonotrile, dissolved on dry acetonitrile (2 mL) and added. A solution of tetrazole in dry acetonitrile (0.25 M, 2.64 mL) was added under nitrogen and the mixture was stirred at room temperature for 1 hour. 5.5 mL of an 1₂-solution (0.1 M in THF/Lutidine/H₂O 7:2:1) was added and the mixture was stirred an additional 1 hour. The reaction mixture was diluted with ethyl acetate (20 mL) and washed with 2% aqueous sodium sulfite until the iodine colour disappeared. The organic phase was dried (Na₂SO₄), and solvent removed in vacuo. The residue was dissolved in 80% aqueous acetic acid (5 mL) and stirred at room temperature overnight. Solvent was removed in vacuo and the crude material was added diethyl ether (25 mL) and water (10 mL). The water phase was collected and water removed in vacuo. Product was purified on reverse phase preparative HPLC C-18 colum, gradient 0-40% acetonitrile containing 0.1% TFA to give the tert-butyl-protected 2. generation branched polymer product.

HPLC-MS: m/z=1171 (M+Na); 1149 (M+), 1093 (lost of tert-butyl in the MS) Rₜ=2.76 min.

Deprotection of β-cyanoethyl groups and removal of tert-butyl ester group, is subsequently done using conventional base and acid treatments as known to the person skilled in the art.

Attachment to Peptides

### Example 51

Conjugation to polypeptides with internal ortogonal Dde protected ε-lysin residue tethered to a solid support:

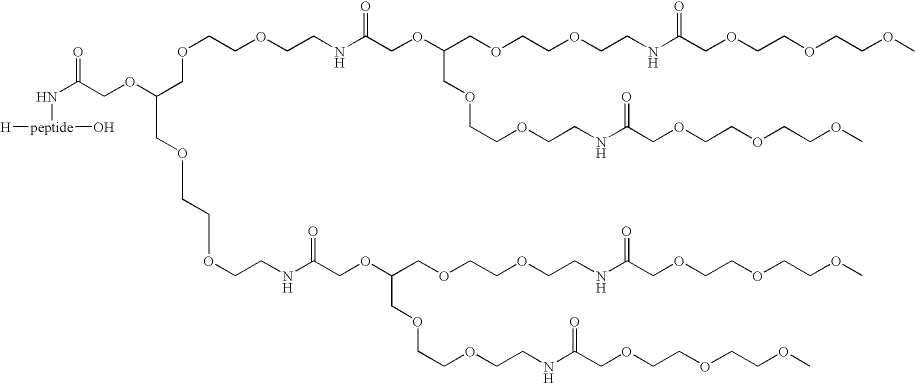

The polypeptide is assembled on a solid support using standard Fmoc peptide chemistry with conventional Fmoc protected amino acids, and standard coupling reagents. On an appropriate location in the linear sequence, an ortogonal Dde ε-protected lysine residue is introduced. When the primary peptide sequence is completed, the terminal Fmoc-protection group is left on. The ortogonal Dde ε-protected lysine residue is deprotected using 2% hydrazine in DMF as described in Novabiochem (2002-2003 catalogue, synthesis notes p. 4.12). A second generation branched polymer is builded using the procedure described in example 11, step 2-8. The final cleaved product is further purified using preparative HPLC.

### Example 52

General example of conjugation to polypeptides in solution: The dendritic polymer prepared as described above is converted into its N-hydroxysuccinimide ester, using TSTU as described in the above examples. The N-hydroxysuccinimide ester activated polymer is then added to an appropriate buffer solution (such as 0.1 M phosphate buffer pH 7.0) containing the polypeptide to be derivatised. The reaction mixture is stirred for one hour at room temperature. The polypeptide conjugate is then purified by the best suited technique, including but not limited to HPLC, ion exchange chromatography, size exclusion chromatography, dialysis ect. Products can subsequently be characterised by MALDI-TOF, LC-MS or equivalent techniques to determine the extent of polymer conjugation.

### Example 53

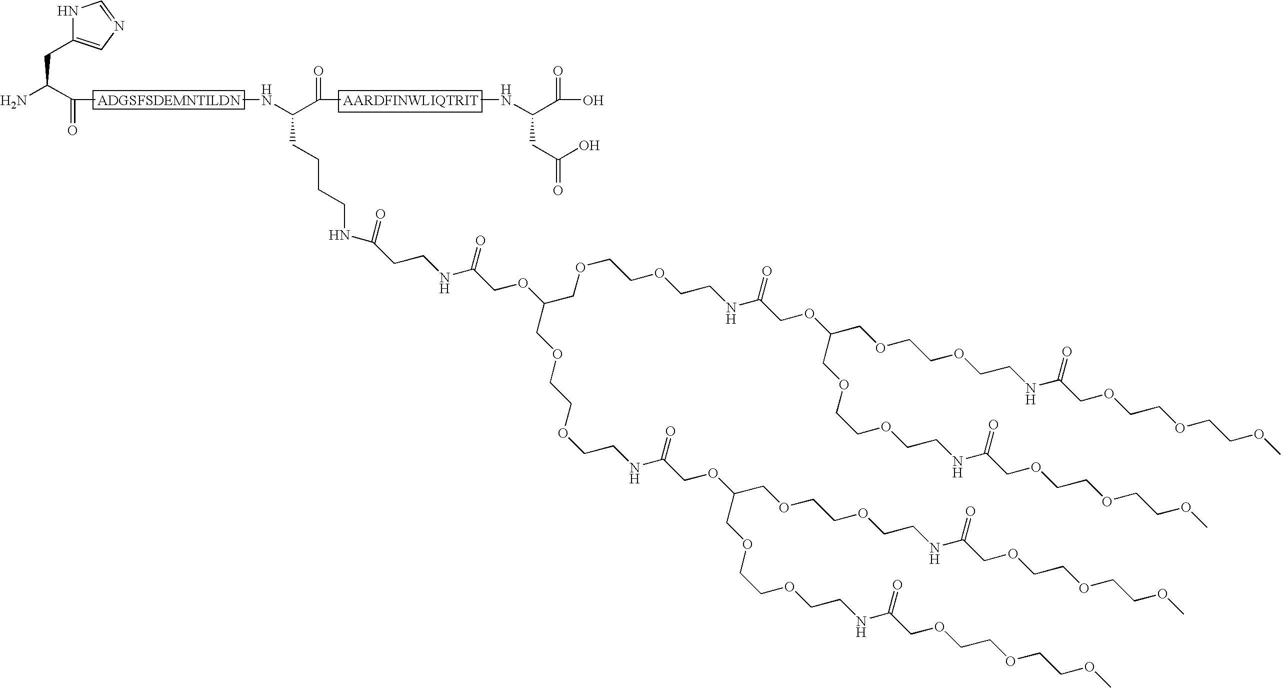

L17K, K30R GLP-2 (1-33) (36 mg; 10 mmol) was dissolved in water (2.3 ml) and cooled on an ice bath to 4° C. pH was adjusted to 12.1 with 1N NaOH solution. The solution was then stirred for 2 min. at 8° C. pH was lowered to 9.5 using 1M aqueous acetic acid, and cold NMP (5 ml) was added. The peptide solution was then stirred at 10° C., while pH was raised to 11.5 by addition of triethyl amine. The temperature was raised to 15° C., and a solution of N-hydroxysuccinimidyl 3-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)-acetylamino)propanoate (19.8 mg; 11 mmol) in NMP (1 ml) was added. The mixture was stirred at 15° C. for 20 min. Then a solution of glycine (0.47 ml, 100 mg/ml) was added. pH was adjusted to 8.5 using 5M aqueous acetic acid solution. The reaction mixture was filtered, and to filtrate was added water to a total volume of 18 ml. The product was purified on preparative HPLC using a C18 column using a linear gradient (30->55%) of acetonitrile water. Pure samples were pooled, diluted with water and freeze dried.

Yield: 3.8% (8%). LC-MS: (m/4)+1=1361; (m/5)+1=1089; (m/6)+1=907. Rt=3.28 min.

### Example 54

Asialo rFVIIa (10.2 mg, 0.2 mmol) in 13.5 ml TRIS buffer (10 mM Cacl2, 10 mM TRIS, 50 mM NaCl, 0.5% Tween 80, pH 7.4) was cooled on an icebath. A solution of N-(4-aminoxybutyl) 3-(1,3-bis{2-(2-[2-(1,3-bis[2-(2-{2-[2-(2-methoxyethoxy)ethoxy]acetamino}ethoxy)ethoxy]-propan-2-yloxy)acetylamino]ethoxy)ethoxy}propan-2-yloxy)acetylamino)propanamide (17 mg; 10.0 mmol, 50×) in 2.5 ml TRIS buffer was added, followed by a solution of galactose oxidase (135 U) and catalase (7500 U) in 2.5 ml TRIS buffer. The reaction mixture was shaken gently for 48 h at 4° C. The slightly unclear solution was then filtered through a 0.45 um filter (Sartorius Minisart®). The buffer was then exchanged to MES (10 mM CaCl₂, 10 mM MES, 50 mM NaCl, pH 6.0) using a NAP-10 columns (Amersham). The mixture was then cooled on ice, and an aqueous solution of EDTA (3.5 ml, 100 mM, pH 8.0, equivalent to [Ca²⁺]) was added. pH was adjusted to 7.6 by addition of 1 M aqueous NaOH, and the sample (6.8 mS/cm) was loaded on a 5 ml HiTrap-Q HP ion-exchange column (Amersham-Biosciences), equilibrated with 10 mM Tris, 50 mM NaCl, pH 7.4. The column was eluted with 10 mM Tris, 50 mM NaCl, pH 7.4 (10 vol, flow: 1 ml/min). The elution buffer was then changed to 10 mM Tris, 50 mM NaCl, 25 mM CaCl₂, pH 7.4 (10 vol, flow: 1 ml/min). The eluates were monitored by UV, and each fraction containing protein was analysed by SDS-PAGE gel electrophoresis. Pure samples of N-glycan modified rFVII were pooled and stored at −80° C.

1. A conjugate comprising a mono disperse branched polymer covalently attached to a peptide.

2. A conjugate represented by the formula:
((branched polymer)-(L3)₀₋₁)_{z}-(peptide),
wherein the L3 is a linking moiety, and z is an integer ≧1 representing the number of branched polymers conjugated to the peptide.

3. A conjugate according to claim 2, wherein L3 is valence bonds or a divalent radical.

4. A conjugate according to claim 3, wherein L3 wherein the divalent radical is alkylene, alkenylene, alkynylene, divalent aromatic group, divalent partly or fully saturated cycloalkyl group, sulfur or oxygen atom, alkyleneoxy, alkylenethio, alkenyleneoxy, alkenylenethio, alkynyleneoxy or alkynylenethio; or N-(4-acetylphenyl)malimide, succimidyl ester activatede malimido derivatives such as succimidyl 4-malimidobutanoate or 1,6-bismalimidohexanes.

5. A conjugate according to claim 2, wherein L3 is
1,2-ethandiyl, 1,3-propandiyl, 1,4-butandiyl, 1,5-pentandiyl, 1,6-hexandiyl, (CH₂CH₂O—)ₙ, where n is an integer between 0 and 10,
—(CR¹R²—CR³R⁴—O)ₙ—, where n is an integer between 0 and 10 and
R¹, R², R³ and R⁴ independently can be hydrogen or C₁₋₆alkyl:
((CH₂)ₘO)ₙ—, where m is 2, 3, 4, 5, 6, and n is an integer between 0 and 10,
or succimidyl 4-malimidobutanoate or 1,6-bismalimidohexanes.

6. A conjugate according to claim 1, wherein the branched polymer has more than one centrally branching point.

7. A conjugate according to claim 1, comprising a branched polymer having a molecular weight of above 1 kDa.

8. A conjugate of claim 7, wherein the branched polymer has a molecular weight of above 3 kDa.

9. A conjugate of claim 8, wherein the branched polymer has a molecular weight of above 5 KDa.

10. A conjugate of according to claim 1, wherein the branched polymer has a molecular weight of below 10 kDa.

11. A conjugate of claim 10, wherein the branched polymer has a molecular weight of below 7 kDa.

12. A conjugate comprising a branched polymer according to claim 1, having an isoelectric point between 3 and 7.

13. A conjugate comprising a branched polymer according to claim 1, having a net negative charge under physiological conditions.

14. A conjugate according to claim 1, wherein the branched polymer is attached to the peptide through an amino acid side chain and/or via the N- and/or C-terminal.

15. A conjugate according to claim 1, wherein the branched polymer is attached to the peptide through a side chain of a derivatised amino acid.

16. A conjugate according to claim 1, wherein the branched polymer is attached to the peptide through a side chain of a non-natural amino acid.

17. A conjugate according to claim 1, wherein the branched polymer is attached to the peptide via an residue which has been amended, added, derivatised and/or substituted into the peptide.

18. A conjugate according to claim 14, wherein the branched polymer is attached to the peptide via a glycan moiety.

19. A conjugate according to any of the claim 18, wherein the branched polymer is attached to the peptide via a modified glycan moiety.

20. A conjugate according to claim 19, wherein the branched polymer is attached to the glycan moiety of a glycoprotein via aldehyde functionalities obtained by selective oxidation of the glycan moiety.

21. A conjugate according to claim 20 wherein the branched polymer is attached to the peptide via an oxidised N- or O-glycan moiety.

22. A conjugate according to claim 1, wherein the branched polymer is attached via reactive aldehydes on the peptide and oxime, hydrazine or hydrazide on the branched polymer

23. A conjugate according to claim 1, comprising a branched polymer together with any of the following peptides: aprotinin, tissue factor pathway inhibitor or other protease inhibitors, insulin or insulin precursors, human or bovine growth hormone, interleukin, glucagon, GLP-1, GLP-2, IGF-I, IGF-II, tissue plasminogen activator, transforming growth factor α or β, platelet-derived growth factor, GRF (growth hormone releasing factor), immunoglubolines, EPO, TPA, protein C, blood coagulation factors such as FVII, FVIII, FIV and FXIII, exendin-3, exentidin-4, and enzymes or functional analogues thereof.

24. A conjugate according to claim 23, wherein the peptide is GLP-1 or FVII.

25. A conjugate of claim 2, wherein z is 1 or 2.

26. A method for preparing a conjugate according to claim 1, wherein the branched polymer is attached to the peptide via the N- and/or C-terminal, and/or an amino acid residue in the peptide.

27. A method according to claim 26, wherein the branched polymer is attached to the peptide through an amino acid side chain.

28. A method according to claim 26, wherein the branched polymer is attached to the peptide via a residue which has been amended, added, derivatised and/or substituted into the peptide.

29. A method according to claim 28, wherein the branched polymer is attached to the peptide through a side chain of a derivatised amino acid.

30. A method according to claim 29, wherein the side chain is an oxidised HO-group in the peptide.

31. A method according to claim 26, wherein the branched polymer is attached to the peptide through a side chain of a non-natural amino acid.

32. A method according to claim 26 wherein the branched polymer is attached to the peptide via a glycan moiety.

33. A method according to claim 32, wherein the branched polymer is attached to the peptide via a modified glycan moiety.

34. A method according to claim 33, wherein the branched polymer is attached to the peptide via an oxidised N-glycan moiety.

35. A method according to claim 34, wherein the branched polymer is attached to the peptide by exposing the glycan moities and then oxidising the glycan moiety.

36. A method according to claim 26, wherein the conjugate is prepared chemo enzymatically, by attaching the branched polymer to a glycosyl transferase activated sugar substrate, and reacting said branched polymer attached sugar substrate with an appropriate glycoprotein using glycosyl transferase catalysis.

37. A branched polymer according to claim 1, made from monomers of the general formula
A-L¹-X-(L 2-B)ₙ
wherein
A and B are points of attachment,
L¹ and L² represents optional linkers,
X is the branching point with n branches.

38. A branched polymer according to claim 37, wherein the branched polymer is built from identical monomers.

39. A branched polymer according to claim 37, wherein the branched polymer is built from two or more different monomers.

40. A branched polymer according to claim 37, wherein the branched polymer is end-capped.

41. A branched polymer according to claim 37, wherein X is linear.

42. A branched polymer according to claim 41 wherein X is a divalent organic radical such as alkylene, alkenylene, alkynylene, divalent aromatic group, divalent partly or fully saturated cycloalkyl group, sulfur or oxygen atom, alkyleneoxy, alkylenethio, alkenyleneoxy, alkenylenethio, alkynyleneoxy or alkynylenethio.

43. A branched polymer according to claim 37, wherein X is a multifunctionalised aryl, alkyl or aryl-alkyl group optionally containing one or more heteroatoms.

44. A branched polymer according to claim 43, wherein X is a multifunctionalised aryl-, alkyl- or aryl-alkyl group containing up to 18 carbon atoms optionally containing one or more heteroatoms.

45. A branched polymer according to claim 44, wherein X is a multiply-functionalised aryl-, alkyl- or aryl-alkyl group containing 1-10 carbon optionally containing nitrogen, oxygen or sulfur.

46. A branched polymer according to claim 43, wherein X is a multivalent organic radical linker represented by multivalent organic radicals such as alkyl-triyl, alkenyl-triyl, alkynyl-triyl, benzentriyl, N,N-trialkylene, cycloalkyl-triyl, benzen-tetrayl, cycloalkyl-tetrayl.

47. A branched polymer according to claim 42 wherein X is a multivalent organic radicals such as propan-1,2,3-triyl, benzen-1,3,4,5-tetrayl, 1,1,1-nitrogentriyl.

48. A branched polymer according to claim 37, wherein X is N.

49. A branched polymer according to claim 41, wherein X is any of the structures:

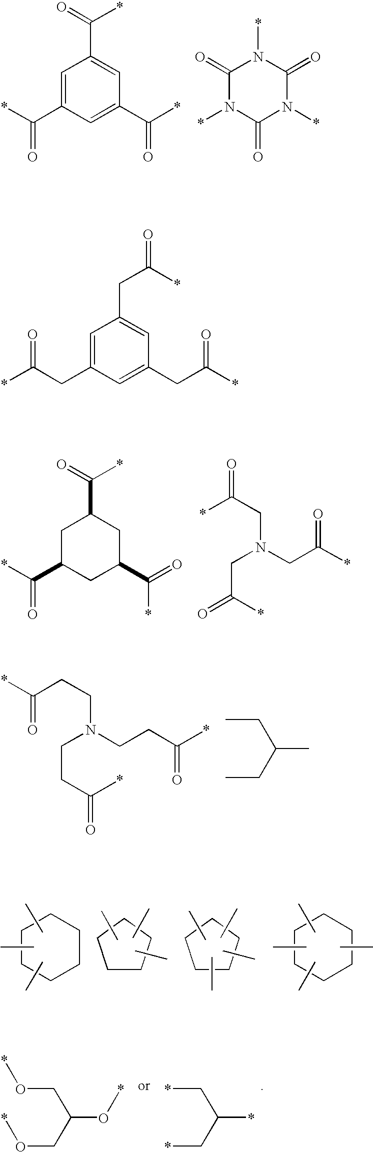

50. A branched polymer according to claim 49, wherein X is

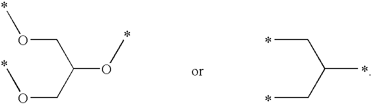

51. A branched polymer according to claim 37, wherein A is a group suitable for formation of covalent bonds.

52. A branched polymer according to claim 37, wherein A is a group capable of reacting with nucleophiles or groups which may be activated to react with nucleophiles.

53. A branched polymer according to claim 37, wherein A is a group suitable for forming amide, carbamate, ester, phosphate ester, thiophosphate ester, phosphoramidates, ether, thioether, oxime, hydrazone, thiazolidine, thioester, alkenyl or alkyl bonds.

54. A branched polymer according to claim 37, wherein A is a group of the formula: COOH, COOR, OCOOR, OP(NR₂)OR, O═P(OR)₂, S═P(OR)(OR′), S═P(SR)(OR′), S═P(SR)(SR′), COCl, COBr, OCOBr, CHO, Br, Cl, I, OTs, OMs, P(OR)₃, alkynes and azides, a p-nitrophenyl carbonate, succinimidyl carbonate, carbonylimidazole, carbonylchlorides, azlactone, cyclic imide thione, isocyanate or isothiocyanates, wherein R and R′ represents is C₁₋₆-alkyl, aryl or substituted aryl.

55. A branched polymer according to claim 54, wherein A is a group of the formula: COOH, COOR, OCOOR, O═P(NR₂)OR, O═P(OR)₂, S═P(OR)(OR′), S═P(SR)(OR′), S═P(SR)(SR′), COCl, COBr, OCOCl, OCOBr, CHO, Br, Cl, I, OTs, OMs, alkynes and azides, wherein R and R′ represents is C₁₋₆-alkyl, aryl or substituted aryl.

56. A branched polymer according to claim 37, wherein A when attached to a peptide or to the group B, is a group of the formula: 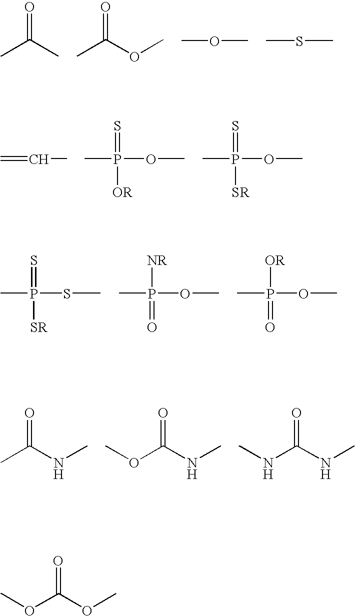
and R represents alkyl, aryl or substituted aryl,
or A is a bond.

57. A branched polymer according to claim 56, wherein the covalent bond formed between A and B, are amide bonds, carbamate bonds, carbonate bonds, ester bonds, phosphate ester bonds, thiophosphate ester bonds, ether bonds, thioether bonds or phosphoramidates.

58. A branched polymer according to claim 37, wherein B is NH₂, OH, N₃, NHR′, OR′, O—NH₂, alkynes, or any of the following
—NH—NH₂, —O—C(O)—NH—NH₂, —NH—C(O)—NH—NH₂, —NH—C(S)—NH—NH₂, —NHC(O)—NH—NH—C(O)—, NH—NH₂, —NH—NH—C(O)—NH—NH₂, —NH—NH—C(S)—NH—NH₂, —NH—C(O)—C₆H₄—NH—NH₂, —C(O)—NH—NH₂ and oxylamine derivatives, such as —C(O)—O—NH₂, —NH—C(O)—O—NH₂ and —NH—C(S)—O—NH₂, and R′ represents H or a protection group.

59. A branched polymer according to claim 58, wherein B is —NH₂, —OH, —N₃, —NHR′, —OR′, —O—NH₂, —Br and R′ represents H or a protection group.

60. A branched polymer according to claim 59, wherein R′ is a protection group of the formula

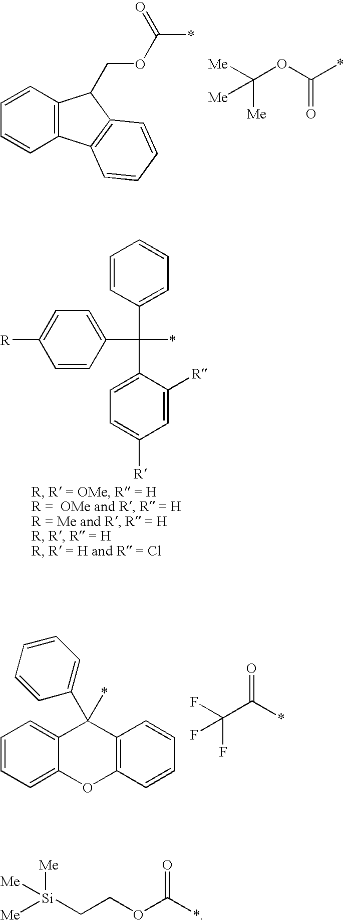

61. A branched polymer according to claim 37, wherein L¹ and L² are valence bonds or divalent radicals.

62. A branched polymer according to claim 61, wherein the L¹ and L² are independently selected from the divalent radicals alkylene, alkenylene, alkynylene, divalent aromatic group, divalent partly or fully saturated cycloalkyl group, sulfur or oxygen atom, alkyleneoxy, alkylenethio, alkenyleneoxy, alkenylenethio, alkynyleneoxy or alkynylenethio;

63. A branched polymer according to claim 62, wherein the divalent radical is selected from
1,2-ethandiyl, 1,3-propandiyl, 1,4-butandiyl, 1,5-pentandiyl, 1,6-hexandiyl, (CH₂CH₂O—)ₘ, where m is an integer between 0 and 10,
—(CR¹R² CR³R⁴—O)ₘ—, where m is an integer between 0 and 10 and
R¹, R², R³ and R⁴ are independently selected from H, or C₁₋₆-alkyl.
—((CH₂)ₙO)ₘ—, where n is 2, 3, 4, 5, 6 and m is an integer between 0 an 10.

64. A branched polymer according to claim 63, wherein L¹ and/or L² represent 1 to 5 (—CH₂CH₂O—) groups.

65. A branched polymer according to claim 61, wherein L¹ is -oxy- or -oxymethyl-, and L² is

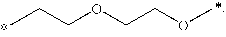

66. A branched polymer according to claim 62, wherein one or both of L¹ and L² are valence bonds.

67. A branched polymer according to claim 37, where the branched polymer is prepared from any of the following monomeric building blocks:

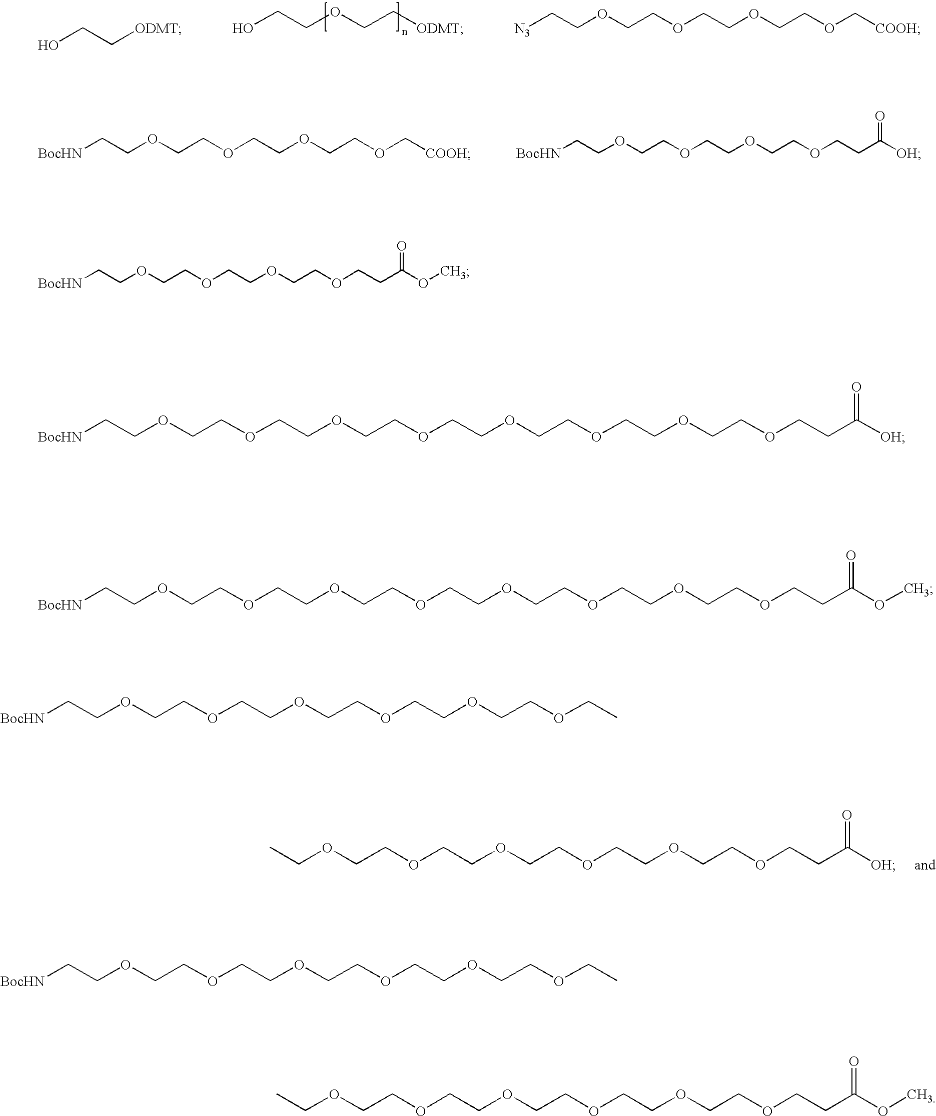

68. A branched polymer according to claim 37, where said branched polymer is being prepared from any of the following monomeric building blocks:

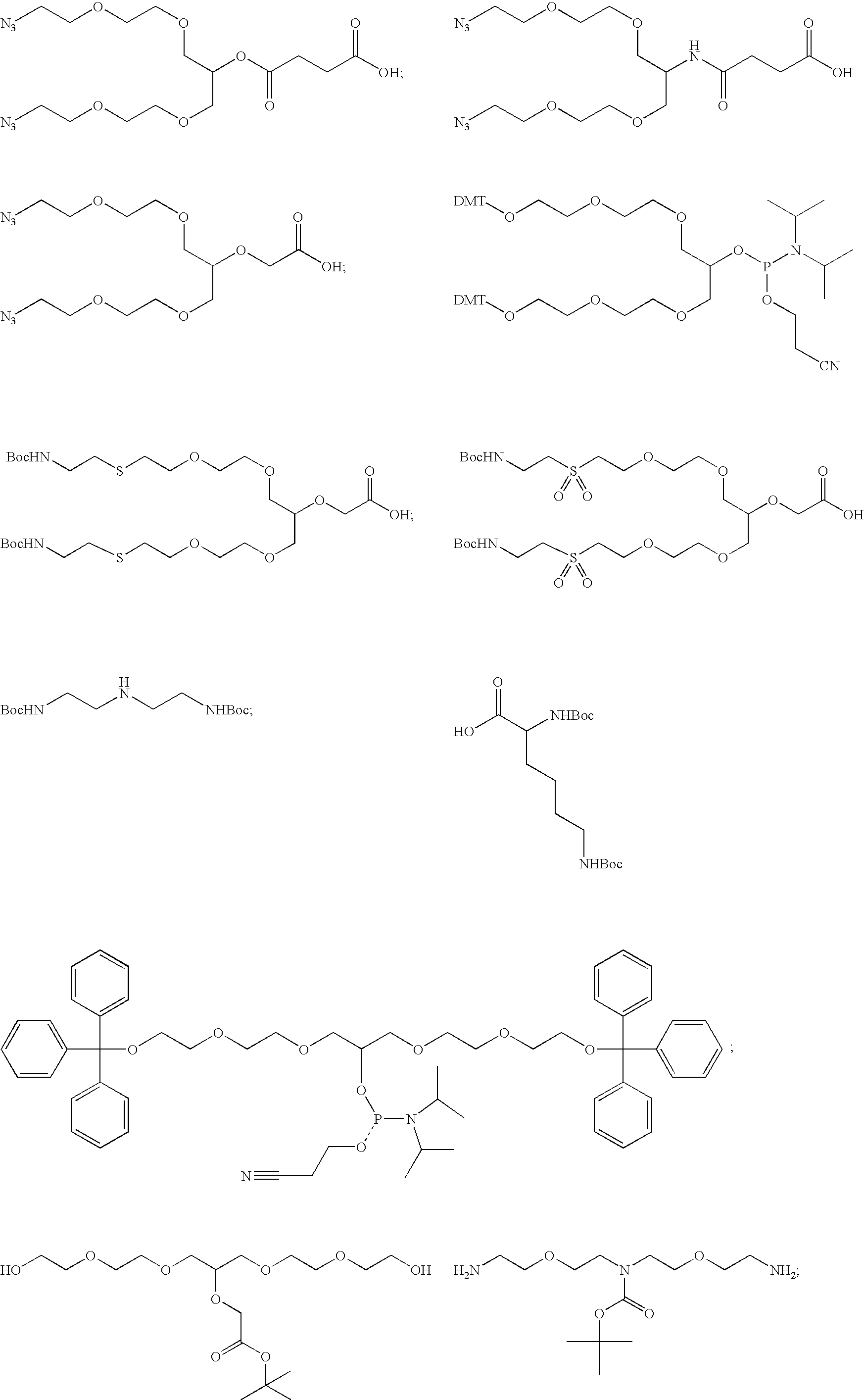

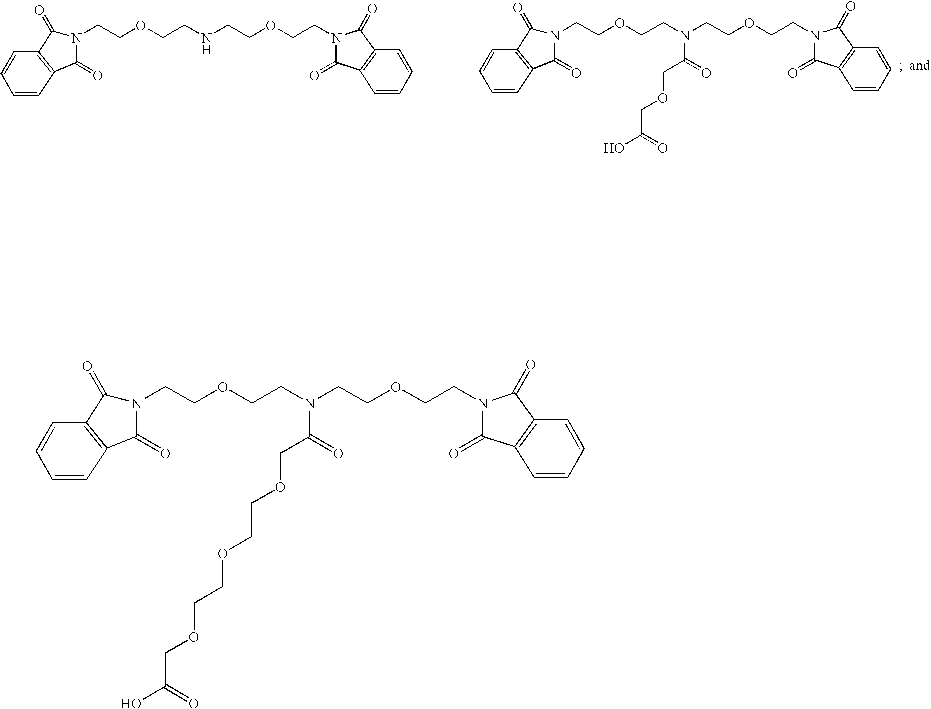

69. A branched polymer according to claim 37, where said branched polymer is being prepared from any of the following monomeric building blocks:

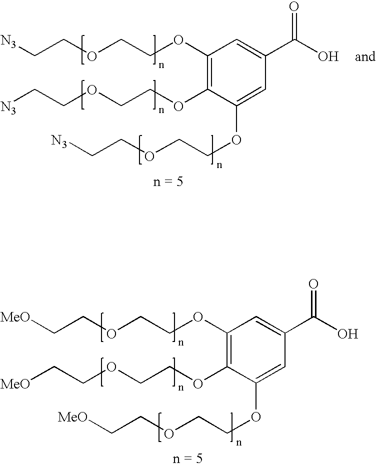

70. A branched polymer according to claim 37, where said branched polymer contains the linker 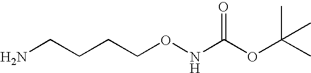
with the provisio, that the branched polymeric product not will contain (CH₂CH₂O)ₙ, wherein n>15.

71. A method for preparation of a branched polymer of claim 37, comprising attaching a monomer of the general formula
A-L¹-X-L²-B′
wherein B′ denotes a protected B, in one or more steps to a solid support,
deprotecting B′ to B,
coupling a suitable A′-L¹-X-L^{2′}-B′ to the solid support, wherein B′ is a protected B and A′ is an optionally activated form of A,
the steps b) and c) are repeated n times to obtain a branched polymer according to claim 38 optionally including a deprotection step after c).

72. A method of producing a branched polymer of claim 37, comprising the steps of reacting a A′-L¹-X-L²-B
wherein A′ denotes a protected A
with a suitable monomer A*-L¹-X-L²-B′, wherein A* denotes an optionally activated form of A and B′ denotes a protected B.
